(19) United States
(12) Patent Application Publication
(10) Pub. No.: US 2017/0368089 A1
(43) Pub. Date: Dec. 28, 2017

(54) COMPOSITION FOR PREVENTING OR TREATING COLITIS

(71) Applicant: UNIVERSITY-INDUSTRY COOPERATION GROUP OF KYUNG HEE UNIVERSITY, GYEONGGI-DO (KR)

(72) Inventors: DONG-HYUN KIM, SEOUL (KR); MYUNG JOO HAN, SEOUL (KR); SE EUN JANG, SEOUL (KR)

(21) Appl. No.: 15/689,124

(22) Filed: Aug. 29, 2017

Related U.S. Application Data
(62) Division of application No. 14/421,770, filed on May 4, 2015.

(30) Foreign Application Priority Data
Aug. 17, 2012 (KR) 10-2012-0089990
Oct. 5, 2012 (KR) 10-2012-0110409
Apr. 23, 2013 (KR) 10-2013-0044990

(51) Int. Cl. A61K 31/7048; A61K 36/718; A61K 35/64; A61K 31/4745; A61K 31/704; A61K 31/7032; A61K 36/88; A61K 36/344

(52) U.S. Cl. CPC A61K 31/7048; A61K 36/88; A61K 36/718; A61K 36/344; A61K 31/4745; A61K 31/704; A61K 31/7032; A61K 35/64

(57) ABSTRACT

The present invention relates to a novel use of an Anemarrhena asphodeloides Bunge extract and the like, and provides a composition for preventing or treating colitis, including an Anemarrhena asphodeloides Bunge extract, a fraction thereof or a compound isolated therefrom as an active ingredient. In addition, the present invention relates to a novel use of a combined extract of medicinal herbs, and provides a composition for preventing or treating colitis, including an Anemarrhena asphodeloides Bunge extract, a Galla Rhois extract, a Codonopsis lancellata extract, and the like as active ingredients, or including an Anemarrhena asphodeloides Bunge extract, a Coptis japonica Makino extract, and the like as active ingredients. According to the present invention, the Anemarrhena asphodeloides Bunge extract and the like or the combined extract can be used as a food or medical materials constituting a pharmaceutical composition or a nutraceutical composition. The pharmaceutical composition or the nutraceutical composition can be used for preventing or treating acute colitis or chronic colitis, and particularly, can effectively prevent, delay, alleviate or treat inflammatory bowel disease (IBD), irritable bowel syndrome (IBS), and the like.

### CROSS REFERENCE TO RELATED APPLICATIONS

This Application is a division of U.S. patent application Ser. No. 14/421,770, filed May 4, 2015, which is a 371 Application of International Application No. PCT/KR2013/007309, filed Aug. 14, 2013, which claims the benefit of Korean Patent Application No. 10-2012-0089990, filed Aug. 17, 2012, Korean Patent Application No. 10-2012-0110409, filed Oct. 5, 2012, and Korean Patent Application No. 10-2013-0044990, filed Apr. 23, 2013, the disclosures of which are incorporated by reference.

### TECHNICAL FIELD

The present invention relates to a novel use of an Anemarrhena asphodeloides Bunge extract and the like, and more particularly, a composition for preventing or treating colitis, including an Anemarrhena asphodeloides Bunge extract, a fraction thereof or a compound isolated therefrom.

### BACKGROUND ART

Colitis is a disease with an inflammation of the colon, occurs due to various causes, has major symptoms, such as, tenesmus, abdominal distension, hypogastric pain, and diarrhea, and in some cases, may exhibit mucous stool, pus faces, or bloody excreta. Colitis may be largely classified into infectious colitis and non-infectious colitis according to causes or may be largely classified into acute colitis and chronic colitis according to the period of the outbreak. As acute colitis, there is pseudomembranous enteritis, and the like by amoebic dysentery, bacillary dysentery, Salmonella, or antibiotics, and as chronic colitis, there is ulcerative colitis, and the like by a Crohn's disease, tuberculosis, syphilis, or X-rays. In addition, colitis includes irritable bowel syndrome (IBS) as well as inflammatory bowel disease (IBD). For the ulcerative colitis (UC) and Crohn's disease (CD) that are typical diseases among the inflammatory bowel disease (IBD), the causes thereof are not yet clearly found, the symptoms thereof may be severe chronic diarrhea and bloody diarrhea together with abdominal pain, and the ulcerative colitis (UC) and Crohn's disease (CD) are a long-lasting disease, and the improvement and deterioration are repeated. In addition, the ulcerative colitis is a disease that continuously forms erosion or ulceration on colon mucosa, and has bloody excrement, mucous and bloody stool, diarrhea, and abdominal pain, and in a severe case, has systemic symptoms, such as, fever, a loss in weight, and anemia. In addition, the ulcerative colitis may occur even in any part of gastrointestinal tract. The Crohn's disease is a disease that discontinuously causes lesion, such as, an ulcer in arbitrary regions of an alimentary canal, and has abdominal pain, diarrhea, and bloody excrement, and also in a severe case, symptoms, such as, fever, bloody discharge, a loss in weight, systemic boredom, and anemia. For the ulcerative colitis and Crohn's disease, there is a difference between the both in lesions and inflammatory conditions, but there are also similar aspects in many points. Therefore, these diseases cannot be often clearly distinguishable.

Conventionally, it has been known that the incidences of the ulcerative colitis and Crohn's disease are high in westerner, but recently, due to the change of life habit, such as, dietary habits, the number of patients with these diseases is rapidly increasing in the Orient including Republic Korea. However, because of unclear causes, no fundamental treatment has been established. For this reason, the medicine, which does not aim to completely treat the diseases, but can alleviate the symptoms thereof, and then, can maintain the alleviated condition for a long period of time, is in use. As a medicine for this symptomatic treatment, an aminosalicylic acid medicine, an adrenal corticosteroid medicine, and an immunosuppressant medicine are mostly used, but various side effects have been reported. For example, it has been reported that Salazosulfapyridine that is often used as the aminosalicylic acid medicine has side effects, such as, nausea, vomiting, anorexia, rash, headache, a liver disease, leucopenia, red cell anomaly, proteinuria, and diarrhea. In addition, as the adrenal corticosteroid medicine, generally, prednisolone is used in an oral administration, enema, suppository, and an intravenous injection, but has strong side effects, such as, stomach ulcer or femoral head necrosis due to a long period use. However, when being stopped taking the medicine, a recurrence of the symptom may occur, and thus, the medicine should be continuously taken. Therefore, the development of the medicine for treating a bowel disease, such as, an ulcerative colitis and a Crohn's disease is being demanded, in which the medicine has excellent effect, is safe, and has no side effects. Similarly, an irritable bowel syndrome (IBS) is chronic celiopathy, in which the cause thereof is not clear. Currently, a fundamental medicine for treating the IBS is not developed, and the symptomatic treatment is being performed to alleviate the symptoms of each of the types. For example, an anticholinergic drug having a spasmolytic function that suppresses the contraction of a smooth muscle is used for a diarrhea IBS, saline cathartics are used for a constipation IBS, and it is difficult to control an alternative IBS by using a medicine, and a medicine for enhancing the movement of alimentary tract is fundamentally used for the alternative IBS.

Meanwhile, Anemarrhena asphodeloides Bunge is a perennial plant that belongs to a monocotyledon liliales Anemarrhena asphodeloides Bunge, and the root stock of the Anemarrhena asphodeloides Bunge that is dried with the skin thereon or the rhizome thereof is used as a medicine. It has been reported that the rhizome of the Anemarrhena asphodeloides Bunge is used as a fever reducer in a field of oriental medicine, and has an effect on chronic bronchitis or diabetes. With reference to the pharmaceutical use of the Anemarrhena asphodeloides Bunge, Korean Patent No. 10-0856335 discloses that the compound isolated from the Anemarrhena asphodeloides Bunge has an effect on preventing and treating a respiratory disease; Korean Patent No. 10-0923953 discloses that an Anemarrhena asphodeloides Bunge extract has an effect on alleviating the damage of choline nervous system; and Korean Patent No. 10-1075742 discloses that the compound isolated from the Anemarrhena asphodeloides Bunge has an effect on preventing and treating a lipid metabolic disease.

In addition, Galla Rhois is a cocoon, which is made by parasitizing Schlechtendalia chinensis Bell that belongs to Homoptera aphid on the leaves of Rhus javanica L. that belongs to Anacardiaceae. In the field of oriental medicine, the cocoon without insects is called Galla Rhois, and is used as a medicine. It had been reported that as the medicine action of the Galla Rhois, there are a convergence effect, an anti-microbial function, an antibiotic function, an action on protecting a liver function, and an anti-oxidative activity. Korean Patent No. 10-0588470 discloses that a Galla Rhois extract has an effect on suppressing the metastasis of cancer cell, and Korean Patent No. 10-1078002 discloses that a Galla Rhois extract has an effect on preventing and treating obesity.

In addition, Codonopsis lanceolata is a perennial climbing plant that belongs to Campanulaceae, and grows wild across Republic Korea. The gross roots of the Codonopsis lanceolata are widely and medicinally used, and it has been reported that the roots of the Codonopsis lanceolata has a medicinal effect, such as, the discharge of phlegm.

In addition, Coptis is an evergreen perennial plant that belongs to dicotyledones ranales Ranunculaceae, and a specific example thereof includes Coptis teeta, Coptis japonica, Coptis chinensis, Coptis deltoidea, and Coptis omeiensis. In the field of oriental medicine, the rhizome thereof is collected and then is dried in the sun so as to be used as a medicine. As the ingredients with the medicinal properties thereof, there have been reported Berberine, Coptisine, Worenine, Palmatine, and the like, which has an effect, such as, an anti-bacterial effect, an anti-inflammatory action, a removal of fever, an action of facilitating the secretion of bile, and an action of lowering blood pressure. With reference to the pharmaceutical use of the Coptis, Korean Laid-Open Patent Publication No. 10-2001-0085669 discloses a pharmaceutical composition for preventing and treating drug addiction, which includes a Coptis extract as an active ingredient; Korean Laid-Open Patent Publication No. 10-2009-0029022 discloses a composition for preventing and treating a skin disease due to UV rays, which includes a Coptis extract as an active ingredient; Korean Laid-Open Patent Publication No. 10-2013-0022733 discloses a composition for treating a pancreatic cancer, which includes a Coptis chinensis extract; Korean Patent No. 10-0970739 discloses a composition for preventing and treating a respiratory disease, which includes a Coptis extract as an active ingredient; and above these, there have been disclosed a composition for treating a periodontal disease, which includes a Coptis extract, a composition for preventing and treating diabetic complications, which includes a Coptis extract, a composition for protecting and regenerating a nerve cell, which includes a Coptis extract, and the like.

However, there are no known effects of an Anemarrhena asphodeloides Bunge that is related to the prevention or treatment of colitis, and also, there are no studies on the pharmaceutical effect for preventing or treating colitis by using an Anemarrhena asphodeloides Bunge in combination with Galla Rhois and Codonopsis lanceolata or using an Anemarrhena asphodeloides Bunge in combination with Coptis.

### SUMMARY

The present invention is induced under this conventional technological background, and an object of the present invention is to provide a use of an Anemarrhena asphodeloides Bunge extract, and the like, for preventing or treating colitis.

In addition, another object of the present invention is to provide a use of a combined extract including an Anemarrhena asphodeloides Bunge extract, a Galla Rhois extract, and a Codonopsis lanceolata extract, for preventing or treating colitis.

In addition, still another object of the present invention is to provide a use of a combined extract including an Anemarrhena asphodeloides Bunge extract and a Coptis extract, for preventing or treating colitis.

The present inventors performed a research for developing an extract having the activity for preventing or treating colitis using, as an object, a lot of natural substances that secure safety as compared with synthesized chemical substances. As a result, the inventors found that an extract obtained from an Anemarrhena asphodeloides Bunge, a fraction thereof, or a compound isolated therefrom has excellent activity for preventing or treating colitis on an animal model with colitis, and thus, completed the present invention. In addition, the present inventors found that a combined extract composed of an Anemarrhena asphodeloides Bunge extract, a Galla Rhois extract, a Codonopsis lanceolata extract, and the like has excellent activity for preventing or treating colitis on an animal model with colitis, and thus, completed the present invention. In addition, the present inventors found that a combined extract composed of an Anemarrhena asphodeloides Bunge extract, a Coptis extract, and the like has excellent activity for preventing or treating colitis on an animal model with colitis, and thus, completed the present invention.

An aspect of the present invention provides a composition for preventing or treating colitis, which includes any one selected from an Anemarrhena asphodeloides Bunge extract, an alcohol-soluble fraction having 3 to 8 carbon atoms of the Anemarrhena asphodeloides Bunge extract, Mangiferin, Neomangiferin, or Timosaponin A-III as an active ingredient.

Another aspect of the present invention provides a composition for preventing or treating colitis, which includes any one selected from an Anemarrhena asphodeloides Bunge extract, an alcohol-soluble fraction having 3 to 8 carbon atoms of the Anemarrhena asphodeloides Bunge extract, Mangiferin, Neomangiferin, or Timosaponin A-III and any one selected from a Gallo Rhois extract, an alcohol-soluble fraction having 3 to 8 carbon atoms of the Gallo Rhois extract, or 1,2,3,4,6-penta-O-galloyl-β-D-glucose, as an active ingredient. In addition, the present invention provides a composition for preventing or treating colitis, which includes any one selected from an Anemarrhena asphodeloides Bunge extract, an alcohol-soluble fraction having 3 to 8 carbon atoms of the Anemarrhena asphodeloides Bunge extract, Mangiferin, Neomangiferin, or Timosaponin A-III and any one selected from a Codonopsis lanceolata extract, an alcohol-soluble fraction having 3 to 8 carbon atoms of the Codonopsis lanceolata extract, or Lancemaside A, as an active ingredient. In addition, the present invention provides a composition for preventing or treating colitis, which includes any one selected from an Anemarrhena asphodeloides Bunge extract, an alcohol-soluble fraction having 3 to 8 carbon atoms of the Anemarrhena asphodeloides Bunge extract, Mangiferin, Neomangiferin, or Timosaponin A-III; any one selected from a Gallo Rhois extract, an alcohol-soluble fraction having 3 to 8 carbon atoms of the Galla Rhois extract, or 1,2,3,4,6-penta-O-galloyl-β-D-glucose; and any one selected from a Codonopsis lanceolata extract, an alcohol-soluble fraction having 3 to 8 carbon atoms of the Codonopsis lanceolata extract, or Lancemaside A, as an active ingredient.

Still another aspect of the present invention provides a composition for preventing or treating colitis, which includes any one selected from an Anemarrhena asphodeloides Bunge extract, an alcohol-soluble fraction having 3 to 8 carbon atoms of the Anemarrhena asphodeloides Bunge extract, Mangiferin, Neomangiferin, or Timosaponin A-III; and any one selected from a Coptis extract or an alcohol-soluble fraction having 3 to 8 carbon atoms of the Coptis extract, as an active ingredient.

The composition for preventing or treating colitis according to the present invention is preferably a pharmaceutical composition or a food composition.

The Anemarrhena asphodeloides Bunge extract according to the present invention; a combined extract composed of an Anemarrhena asphodeloides Bunge extract, a Galla Rhois extract, and a Codonopsis lanceolata extract; or a combined extract composed of an Anemarrhena asphodeloides Bunge extract and a Coptis extract can be used as a food or medical material constituting a pharmaceutical composition or a nutraceutical composition. The pharmaceutical composition or the nutraceutical composition can be used for preventing or treating acute colitis or chronic colitis, and especially, can effectively prevent, delay, alleviate, or treat inflammatory bowel disease (IBD) or irritable bowel syndrome (IBS).

### BRIEF DESCRIPTION OF DRAWINGS

FIG. 1 illustrates Chemical Structural Formulas of Mangiferin and Neomangiferin.

FIG. 2 is the results illustrating the suppressions of the expressions of inflammatory marker substances in the colons of the experimental animal group administered with an n-butanol-soluble fraction of the Anemarrhena asphodeloides Bunge extract obtained in Preparation Example 2 after inducing acute colitis by treating TNBS. In FIG. 2, “JM” refers to an n-butanol-soluble fraction of the Anemarrhena asphodeloides Bunge extract obtained in Preparation Example 2 and “MEL” refers to Mesalazine.

FIG. 3 is the results illustrating the suppressions of the expressions of inflammatory marker substances in the colons of the experimental animal group administered with Mangiferin after inducing acute colitis by treating TNBS. In FIG. 3, “MF” refers to Mangiferin and “MEL” refers to Mesalazine.

FIG. 4 is the results illustrating the suppression of the inflammatory response by Mangiferin in macrophagocyte treated with lipopolysaccharide (LPS) or peptidoglycan (PG).

FIG. 5 is Chemical Structural Formula of glucose1,2,3,4,6-penta-O-galloyl-β-D-glucose.

FIG. 6 is a graph illustrating the change amount of weight in the animal models with acute colitis induced by TNBS when an extract-based sample is administered.

FIG. 7 is a graph illustrating the change amount of weight in the animal models with acute colitis induced by TNBS when a butanol-soluble fraction-based sample is administered.

FIG. 8 is the results illustrating the suppressions of the expressions of inflammatory marker substances in the colons of the experimental animal group administered with an extract-based sample after inducing acute colitis by treating TNBS.

FIG. 9 is the results illustrating the suppressions of the expressions of inflammatory marker substances in the colons of the experimental animal group administered with a butanol-soluble fraction-based sample after inducing acute colitis by treating TNBS.

FIG. 10 is the results illustrating the suppression of inflammatory response by a combined extract composed of a Galla Rhois extract, an Anemarrhena asphodeloides Bunge extract, and a Codonopsis lanceolata extract in macrophagocyte treated with lipopolysaccharide (LPS).

FIG. 11 is the graphs illustrating the weight changes and macroscopic scores of colons in the animal models with acute colitis induced by TNBS when an n-butanol-soluble fraction of the Anemarrhena asphodeloides Bunge extract obtained in Preparation Example 22 is used as a medicine sample and FIG. 12 is the graphs illustrating the results of the measurements of the colon lengths and MPO activity in the animal models with acute colitis induced by TNBS when an n-butanol-soluble fraction of the Anemarrhena asphodeloides Bunge extract obtained in Preparation Example 22 is used as a medicine sample.

FIG. 13 is the results illustrating the results of the measurements of the weight change, the macroscopic score of colon, the colon length, and the MPO activity in the animal model with acute colitis induced by TNBS when Mangiferin obtained in Preparation Example 23 is used as a medicine sample.

FIG. 14 is the results illustrating the weight change and the macroscopic score of colon in the animal model with acute colitis induced by TNBS when a combined extract is used as a medicine sample and FIG. 15 is the results illustrating the results of the measurements of the colon length and the MPO activity in the animal model with acute colitis induced by TNBS when a combined extract is used as a medicine sample.

FIG. 16 is the graphs illustrating the expression amounts of IL-1 beta and IL-6 in the colon tissues of the animal model with acute colitis induced by TNBS when an n-butanol-soluble fraction of the Anemarrhena asphodeloides Bunge extract obtained in Preparation Example 22 is used as a medicine sample and FIG. 17 is the graphs illustrating the expression amounts of TNF-alpha and IL-10 in the colon tissues of the animal model with acute colitis induced by TNBS when an n-butanol-soluble fraction of the Anemarrhena asphodeloides Bunge extract obtained in Preparation Example 22 is used as a medicine sample.

FIG. 18 is the graphs illustrating the expression amounts of proinflammatory cytokine and anti-inflammatory cytokine in the colon tissues of the animal model with acute colitis induced by TNBS when Mangiferin obtained in Preparation Example 23 is used as a medicine sample.

FIG. 19 is the graphs illustrating the expression amounts of IL-1 beta and IL-6 in the colon tissues of the animal model with acute colitis induced by TNBS when a combined extract is used as a medicine sample and FIG. 20 is the graphs illustrating the expression amounts of TNF-alpha and IL-10 in the colon tissues of the animal model with acute colitis induced by TNBS when a combined extract is used as a medicine sample.

FIG. 21 is the results illustrating the suppressions of the expressions of inflammatory marker substances in the colon tissues of the animal model when a combined extract is administered to the animal model with acute colitis induced with TNBS.

FIG. 22 is the graphs illustrating the weight change and microscopic score of the colon in the animal model with acute colitis induced by DSS when a combined extract is used as a medicine sample and FIG. 23 is the graphs illustrating the results of the measurements of the colon length and MPO activity in the animal model with acute colitis induced by DSS when a combined extract is used as a medicine sample.

FIG. 24 is the graphs illustrating the expression amounts of IL-1 beta and IL-10 in the colon tissues of the animal model with acute colitis induced by DSS when a combined extract is used as a medicine sample and FIG. 25 is the graphs illustrating the expression amounts of TNF-alpha and IL-6 in the colon tissues of the animal model with acute colitis induced by DSS when a combined extract is used as a medicine sample.

FIG. 26 is the results illustrating the suppressions of the expressions of inflammatory marker substances in the colon tissues of the animal model when a combined extract is administered to the animal model with acute colitis induced with DSS.

FIG. 27 is the graphs illustrating the weight change and microscopic score of the colon in the animal model with chronic colitis induced by oxazolone when a combined extract is used as a medicine sample and FIG. 28 is the graphs illustrating the results of the measurements of the colon length and MPO activity in the animal model with chronic colitis induced by oxazolone when a combined extract is used as a medicine sample.

FIG. 29 is the graphs illustrating the expression amounts of IL-1 beta and IL-10 in the colon tissues of the animal model with chronic colitis induced by oxazolone when a combined extract is used as a medicine sample and FIG. 30 is the graphs illustrating the expression amounts of TNF-alpha and IL-6 in the colon tissues of the animal model with chronic colitis induced by oxazolone when a combined extract is used as a medicine sample.

FIG. 31 is the results illustrating the suppressions of the expressions of inflammatory marker substances in the colon tissues of the animal model when a combined extract is administered to the animal model with chronic colitis induced with oxazolone.

FIG. 32 is the graphs illustrating the weight change and microscopic score of the colon in the animal model with chronic colitis induced by DSS when a combined extract is used as a medicine sample and FIG. 33 is the graphs illustrating the results of the measurements of the colon length and MPO activity in the animal model with chronic colitis induced by DSS when a combined extract is used as a medicine sample.

FIG. 34 is the graphs illustrating the expression amounts of IL-1 beta and IL-10 in the colon tissues of the animal model with chronic colitis induced by DSS when a combined extract is used as a medicine sample and FIG. 35 is the graphs illustrating the expression amounts of TNF-alpha and IL-6 in the colon tissues of the animal model with chronic colitis induced by DSS when a combined extract is used as a medicine sample.

FIG. 36 is the results illustrating the suppressions of the expressions of inflammatory marker substances in the colon tissues of the animal model when a combined extract is administered to the animal model with chronic colitis induced with DSS.

FIG. 37 is Chemical Structural Formula of Timosaponin A-III.

FIG. 38 is the graph illustrating the weight change of the animal model with acute colitis induced by TNBS when Timosaponin A-III is used in Preparation Example 34 as a medicine sample and FIG. 39 is the graph illustrating the macroscopic score of the colon isolated from the animal model with acute colitis induced by TNBS when Timosaponin A-III is used in Preparation Example 34 as a medicine sample.

FIG. 40 is the results illustrating the colon length of the animal model with acute colitis induced with TNBS when Timosaponin A-III is used in Preparation Example 34 as a medicine sample and FIG. 41 is the results illustrating the measurement of MPO activity of the colon isolated from the animal model with acute colitis induced with TNBS when Timosaponin A-III is used in Preparation Example 34 as a medicine sample.

### DETAILED DESCRIPTION

Hereinafter, the terms used in the present invention will be described.

The term, “prevention” used in the present invention refers to all activities that can suppress the symptoms of a specific disease or can delay the progression of the specific disease by administering the composition of the present invention.

The term, “treatment” used in the present invention refers to all improvement activities that can improve or beneficially change the symptoms of a specific disease by administering the composition of the present invention.

The term, “administration” used in the present invention refers to the supply of the predetermined composition of the present invention to an object in an arbitrary proper method. At this time, the object refers to all kinds of animals, such as human, a monkey, a dog, a goat, a pig, or a rat having the disease, in which the symptoms of a specific disease can be improved by administering the composition of the present invention.

Hereinafter, the present invention will be described in detail.

The present invention provides a composition for preventing or treating colitis. The colitis in the present invention refers to the condition having the inflammation of colon caused due to bacterial infection or pathologic fermentation of intestinal contents, and is a concept including infectious colitis and non-infectious colitis. A specific kind of colitis that can be prevented or treated by the combined extract of the present invention is an inflammatory bowel disease and an irritable bowel syndrome, but the present invention is not limited thereto. Examples of the inflammatory bowel disease include ulcerative colitis or a Crohn's disease. In addition, the colitis that can be prevented or treated by the combined extract of the present invention includes both of acute colitis and chronic colitis. The acute colitis refers to the inflammation in colon or large intestine, which occurs in acute, and mainly has mucus diarrhea or fresh blood symptom because of the damage of mucous membrane caused due to the inflammation. In the present invention, the acute colitis includes acute pseudomembranous colitis and acute ulcerative colitis as well as general acute infective colitis.

The composition for preventing or treating colitis according to an embodiment of the present invention includes any one (hereinafter, referred to as an Anemarrhena asphodeloides Bunge extract, and the like) selected from an Anemarrhena asphodeloides Bunge extract, an alcohol-soluble fraction having 3 to 8 carbon atoms of the Anemarrhena asphodeloides Bunge extract, Mangiferin, Neomangiferin, or Timosaponin A-III as an active ingredient. The Anemarrhena asphodeloides Bunge extract and the like may effectively prevent or treat colitis by making the appearance of the colon to be good, suppressing the contraction of the colon, and maintaining low Myeloperoxidase (MPO) activity in the animal model with colitis induced by 2,4,6-trinitrobenzenesulfonic acid (TNBS).

The Anemarrhena asphodeloides Bunge extract in the present invention may be extracted from various organs or parts, for example, the leaves, flowers, roots, stems, rhizomes, fruits, seeds, and the like of an Anemarrhena asphodeloides Bunge, and preferably may be extracted from the rhizomes thereof. In addition, the Anemarrhena asphodeloides Bunge extract may be prepared using a general extraction method that is known in the related art, for example, a solvent extraction method. The extraction solvents used for the solvent extraction method may be selected from the group consisting of water, low alcohol having 1 to 4 carbon atoms (for example, methanol, ethanol, propanol, and butanol), or the mixture thereof, that is, water-containing low alcohol, propyleneglycol, 1,3-butyleneglycol, glycerin, acetone, diethylether, ethyl acetate, butyl acetate, dichloromethane, chloroform, hexane, and the mixture thereof, and, among them, may be preferably selected from water, alcohol, or the mixture thereof. When water is used as an extraction solvent, the water may be preferably hot water. In addition, when alcohol is used as an extraction solvent, the alcohol may be preferably low alcohol having 1 to 4 carbon atoms, and the low alcohol may be more preferably selected from methanol or ethanol. In addition, when water-containing alcohol is used as an extraction solvent, the content of alcohol may be preferably 50 to 90% and more preferably 60 to 80%. Meanwhile, it is clearly understood by a person skilled in the related art that the Anemarrhena asphodeloides Bunge extract in the present invention may have the effect that is substantially same even if other extraction solvents are used other than the above-described extraction solvents. In addition, the Anemarrhena asphodeloides Bunge extract in the present invention includes the extracts obtained through other general extraction methods or the extracts obtained through a purification and fermentation process, as well as the extracts obtained by the above-described extraction solvents. For example, the extract according to the present invention include the active fractions obtained through various purification and extraction methods that are further performed, for example, the extracts from the fermentation products, which are produced by the supercritical extraction under a high temperature and decompression by carbon dioxide, by the extraction method using ultrasonic wave, by the isolation using an ultra-filtration membrane having a certain molecular weight cutoff value, by the isolation using various chromatography (which are manufactured for the isolation according to sizes, charges, hydrophobicity, or affinity), by using various microorganisms, or in a natural state, and the like. The extraction method by the supercritical extraction under a high temperature and decompression by carbon dioxide refers to a supercritical fluid extraction, and generally, the supercritical fluid has the properties of liquid and gas, which are obtained when gas reaches the critical point under the condition of high temperature and high pressure, and has chemically similar polarity as a non-polar solvent. Due to these properties, the supercritical fluid is used for the extraction of fat-soluble substances (J. Chromatogr. A. 1998; 479:200 to 205). The carbon dioxide becomes the supercritical fluid having the liquid and gas properties together, which is realized by being subjected to the process of making a pressure and temperature to be the critical point by the operation of supercritical fluid instrument, and thus, has increased solubility in a fat-soluble solute. When the supercritical carbon dioxide passes through an extraction container including a sample in a predetermined amount, the fat-soluble substance included in the sample is extracted in the supercritical carbon dioxide. After extracting the fat-soluble substance, the components that are not extracted only with pure supercritical carbon dioxide may be extracted by passing the supercritical carbon dioxide including a small amount of co-solvent through the sample remained in the extraction container, again. The supercritical fluid that is used in the supercritical extraction method of the present invention may effectively extract an active component by using supercritical carbon dioxide or a mixed fluid prepared by further mixing a co-solvent with carbon dioxide. Such a co-solvent may be a mixture of one or two or more types selected from the group consisting of chloroform, ethanol, methanol, water, ethyl acetate, hexane, and diethyl ether. The extracted sample includes carbon dioxide, which is volatilized into the air at room temperature, and thus, the extract obtained by the above-described method may be used as a cosmetic composition and the co-solvent may be removed with a rotary vacuum evaporator. In addition, the ultrasonic extraction method is a method using energy generated by ultrasonic vibrations. The ultrasonic waves may break an insoluble solvent included in the sample in the water-soluble solvent, and at this time, the high local temperature generated allows kinetic energy of reactant particles, which are located around, to be increased, thereby obtaining sufficient energy for the reaction. A pressure is highly induced by the shock effect of ultrasonic energy, and thus, a mixing effect of the solvent and substance included in the sample is increased, thereby increasing extraction efficiency. The extraction solvent that can be used in the ultrasonic extraction method may be a mixture of one or two or more types selected from the group consisting of chloroform, ethanol, methanol, water, ethyl acetate, hexane, and diethyl ether. A extract may be obtained from the extracted sample by a general method for preparing an extract, which includes collecting a filtrate by vacuum-filtering the extracted sample, removing the filtrate with a rotary vacuum evaporator, and then, subjecting the filtrate to a freeze-drying. In addition, the Anemarrhena asphodeloides Bunge extract according to the present invention includes an extract that is subjected to a fermentation process, and an Anemarrhena asphodeloides Bunge fermentation extract may be prepared as follows. An Anemarrhena asphodeloides Bunge is finely pulverized to be about 100 to 500 meshes, and then, 1 to 50 g/L of a general culture medium of microorganism is added and microorganism, such as, yeast strain or lactobacillus is added in the amount of 10,000 to 100,000 cfu/L. As a culturing temperature, a general microorganism temperature, that is, 30 to 37° C. is used. The culture is performed under an aerobic or general anaerobic condition at pH 5 to 7 for about 5 days to 10 days. Since then, the extract may be obtained through an aging and filtration.

In addition, the alcohol-soluble fraction of the Anemarrhena asphodeloides Bunge extract in the present invention may be obtained from an Anemarrhena asphodeloides Bunge extract. At this time, the alcohol used for obtaining the alcohol-soluble fraction of the Anemarrhena asphodeloides Bunge extract may have 3 to 8 carbon atoms, and in consideration of the solubility of the active substance of the Anemarrhena asphodeloides Bunge extract and a decompressed concentration process after the solubilization of the active substance thereof, it may have preferably 3 to 6 carbon atoms, more preferably 3 to 5 carbon atoms, and still more preferably 4 carbon atoms. In the present invention, the alcohol having 4 carbon atoms, which is used for the solubilization of the Anemarrhena asphodeloides Bunge extract, may be n-butanol, sec-butanol, isobutanol, tert-butanol, and the like. In the present invention, the alcohol-soluble fraction of the Anemarrhena asphodeloides Bunge extract is composed of the components capable of being soluble in the alcohol having 3 to 8 carbon atoms among the components included in the Anemarrhena asphodeloides Bunge extract. In this case, the Anemarrhena asphodeloides Bunge extract is preferably extracted with water, the alcohol having 1 to 2 carbon atoms or the mixed solvent thereof. The alcohol-soluble fraction of the Anemarrhena asphodeloides Bunge extract may be obtained by obtaining the Anemarrhena asphodeloides Bunge extract using water, alcohol having 1 to 2 carbon atoms, or the mixture thereof as an extraction solvent, suspending the extract through adding water to the extract, adding the alcohol (for example, butanol) 3 to 8 carbon atoms to the suspension, and then performing the fractionation of the suspension thus obtained.

In addition, in the present invention, the Anemarrhena asphodeloides Bunge extract or the alcohol-soluble fraction thereof is an active substance and includes Mangiferin or Neomangiferin. In the present invention, Mangiferin or Neomangiferin may be used as an active ingredient in the composition for preventing or treating colitis, instead of the Anemarrhena asphodeloides Bunge extract or alcohol-soluble fraction of the Anemarrhena asphodeloides Bunge extract. However, for the active substance included in the Anemarrhena asphodeloides Bunge extract or alcohol-soluble fraction of the Anemarrhena asphodeloides Bunge extract, a type or a content of a specific component may be slightly different according to the extraction methods or fractionation methods.

In addition, in the present invention, Timosaponin A-III may be isolated from an Anemarrhena asphodeloides Bunge. For example, Timosaponin A-III may be prepared by (i) the step of extracting an Anemarrhena asphodeloides Bunge in alcohol, water, or a mixed solvent of alcohol and water, and more preferably in 70% ethanol, and then, being subjected to a decompressed concentration; (ii) the step of obtaining a water fraction by re-suspending the extract produced in water, and then, performing the fractionation with methylene chloride; (iii) the step of obtaining a butanol fraction by performing the fractionation of the water fraction obtained with butanol; (iv) the step of obtaining a methanol-insoluble fraction and a methanol-soluble fraction by washing the precipitate produced after performing the decompressed concentration of the obtained butanol fraction and then inducing re-crystallization in methanol; and (v) the step of obtaining the fraction including Timosaponin A-III as a main ingredient by performing the column chromatography of the obtained methanol-soluble fraction. In the step (V), the column chromatography is preferably performed using a mixed solvent of methylene chloride, methanol, and water in a silica gel column. The mixed solvent has preferably methylene chloride:methanol:water of 7:1:0.5 to 7:3:1.

The composition for preventing or treating colitis according to another embodiment of the present invention includes any one (hereinafter, referred to as “Anemarrhena asphodeloides Bunge extract, and the like) selected from an Anemarrhena asphodeloides Bunge extract, a fraction that is soluble in the alcohol having 3 to 8 carbon atoms among the Anemarrhena asphodeloides Bunge extract, Mangiferin, Neomangiferin, or Timosaponin A-III; and any one (hereinafter, referred to as “Galla Rhois extract, and the like) selected from a Galla Rhois extract, a fraction that is soluble in the alcohol having 3 to 8 carbon atoms among the Galla Rhois extract, or glucose1,2,3,4,6-penta-O-galloyl-β-D-glucose, as an active ingredient. In addition, the composition for preventing or treating colitis according to another embodiment of the present invention includes any one (hereinafter, referred to as “Anemarrhena asphodeloides Bunge extract, and the like) selected from an Anemarrhena asphodeloides Bunge extract, a fraction that is soluble in the alcohol having 3 to 8 carbon atoms among the Anemarrhena asphodeloides Bunge extract, Mangiferin, Neomangiferin, or Timosaponin A-III; and any one (hereinafter, referred to as “Codonopsis lanceolata extract, and the like) selected from a Codonopsis lanceolata extract, a fraction that is soluble in the alcohol having 3 to 8 carbon atoms among the Codonopsis lanceolata extract, or Lancemaside A, as an active ingredient. In addition, the composition for preventing or treating colitis according to another embodiment of the present invention includes any one (hereinafter, referred to as “Anemarrhena asphodeloides Bunge extract, and the like) selected from an Anemarrhena asphodeloides Bunge extract, a fraction that is soluble in the alcohol having 3 to 8 carbon atoms among the Anemarrhena asphodeloides Bunge extract, Mangiferin, Neomangiferin, or Timosaponin A-III; any one (hereinafter, referred to as “Galla Rhois extract, and the like) selected from a Galla Rhois extract, a fraction that is soluble in the alcohol having 3 to 8 carbon atoms among the Galla Rhois extract, or glucose1,2,3,4,6-penta-O-galloyl-β-D-glucose; and any one (hereinafter, referred to as “Codonopsis lanceolata extract, and the like) selected from a Codonopsis lanceolata extract, a fraction that is soluble in the alcohol having 3 to 8 carbon atoms among the Codonopsis lanceolata extract, or Lancemaside A, as an active ingredient.

The composition for preventing or treating colitis according to another embodiment of the present invention includes a complex extract composed of the extract of at least two medicinal herbs, such as, an Anemarrhena asphodeloides Bunge extract and the like and a Galla Rhois extract and the like; the Anemarrhena asphodeloides Bunge extract and the like and a Codonopsis lanceolata extract and the like; or the Anemarrhena asphodeloides Bunge extract and the like, the Galla Rhois extract and the like, and the Codonopsis lanceolata extract and the like. At this time, a mixed ratio of the Anemarrhena asphodeloides Bunge extract and the like, the Galla Rhois extract and the like, and the Codonopsis lanceolata extract and the like, which constitute the combined extract, is not largely limited. For example, the combined extract composed of the extract of three medicinal herbs may be composed of 10 to 40 wt % of the Anemarrhena asphodeloides Bunge extract and the like, 20 to 80 wt % of the Galla Rhois extract and the like, and 10 to 40 wt % of the Codonopsis lanceolata extract and the like, with respect to the total weight of the combined extract. In addition, the molecular weight ratio of the Anemarrhena asphodeloides Bunge extract and the like, the Galla Rhois extract and the like, and the Codonopsis lanceolata extract and the like in the combined extract composed of the extract of three medicinal herbs is preferably 1:8:1 to 4:2:4, and more preferably 2:6:2 to 3:4:3. The composition for preventing or treating colitis according to another embodiment of the present invention may include a combined extract in varied combinations as an active ingredient. For example, the composition for preventing or treating colitis according to another embodiment of the present invention may include, as an active ingredient, the Galla Rhois extract, the Anemarrhena asphodeloides Bunge extract, and the Codonopsis lanceolata extract; an alcohol-soluble fraction of the Galla Rhois extract, the Anemarrhena asphodeloides Bunge extract, and the Codonopsis lanceolata extract; an alcohol-soluble fraction of the Galla Rhois extract and the Anemarrhena asphodeloides Bunge extract, and the Codonopsis lanceolata extract; an alcohol-soluble fraction of the Galla Rhois extract and the Anemarrhena asphodeloides Bunge extract, and an alcohol-soluble fraction of the Codonopsis lanceolata extract; an alcohol-soluble fraction of the Galla Rhois extract, and the Anemarrhena asphodeloides Bunge extract and the Codonopsis lanceolata extract; an alcohol-soluble fraction of the Galla Rhois extract and an alcohol-soluble fraction of the Anemarrhena asphodeloides Bunge extract and the Codonopsis lanceolata extract; an alcohol-soluble fraction of the Galla Rhois extract, an alcohol-soluble fraction of the Anemarrhena asphodeloides Bunge extract, and the Codonopsis lanceolata extract; an alcohol-soluble fraction of the Galla Rhois extract, an alcohol-soluble fraction of the Anemarrhena asphodeloides Bunge extract, and an alcohol-soluble fraction of the Codonopsis lanceolata extract; or Mangiferin (or, Neomangiferin), 1,2,3,4,6-penta-O-galloyl-β-D-glucose, and Lancemaside A. The composition for preventing or treating colitis according to another embodiment of the present invention may effectively prevent or treat colitis by making the appearance of colon to be good, suppressing the contraction of colon, and maintaining Myeloperoxidase (MPO) activity in a low level in the animal model with colitis induced by 2,4,6-trinitrobenzenesulfonic acid (TNBS). For the composition for preventing or treating colitis according to another embodiment of the present invention, the case of including the Anemarrhena asphodeloides Bunge extract and the like and the Galla Rhois extract and the like, the case of including the Anemarrhena asphodeloides Bunge extract and the like and the Codonopsis lanceolata extract and the like, or the case of including the Anemarrhena asphodeloides Bunge extract and the like, the Galla Rhois extract and the like, and the Codonopsis lanceolata extract and the like exhibit excellent colitis-preventing effect or excellent colitis-treating effect because of their synergy effects, as compared with the case of including only the Anemarrhena asphodeloides Bunge extract and the like as an active ingredient.

A combined extract that is an active ingredient in the composition for preventing or treating colitis according to another embodiment of the present invention may be prepared in various methods. For example, the combined extract composed of the extract of three medicinal herbs may be prepared by mixing a Galla Rhois, an Anemarrhena asphodeloides Bunge, and a Codonopsis lanceolata, adding an extraction solvent thereto, and extracting a combined extract; or by mixing a Galla Rhois, an Anemarrhena asphodeloides Bunge, and a Codonopsis lanceolata, adding an extraction solvent thereto, extracting a combined extract, again adding the alcohol having 3 to 8 carbon atoms to the combined extract, and then performing the fractionation thereof. In addition, the combined extract composed of the extract of three medicinal herbs may be prepared by obtaining a Galla Rhois extract, an Anemarrhena asphodeloides Bunge extract, and a Codonopsis lanceolata extract from a Galla Rhois, an Anemarrhena asphodeloides Bunge, and a Codonopsis lanceolata and then mixing these extracts; or by obtaining a Galla Rhois extract, an Anemarrhena asphodeloides Bunge extract, and a Codonopsis lanceolata extract from a Galla Rhois, an Anemarrhena asphodeloides Bunge, and a Codonopsis lanceolata, obtaining an alcohol-soluble fraction of the Galla Rhois extract, an alcohol-soluble fraction of the Anemarrhena asphodeloides Bunge extract, and an alcohol-soluble fraction of the Codonopsis lanceolata extract from the Galla Rhois extract, the Anemarrhena asphodeloides Bunge extract, and the Codonopsis lanceolata extract, and then mixing these fractions. A method of preparing the Galla Rhois extract or the Codonopsis lanceolata extract is the same as the method of preparing the Anemarrhena asphodeloides Bunge extract as described above, and thus, is not provided.

In addition, an alcohol-soluble fraction of the Galla Rhois extract, an alcohol-soluble fraction of the Anemarrhena asphodeloides Bunge extract, and an alcohol-soluble fraction of the Codonopsis lanceolata extract, which constitute the combined extract that is an active ingredient in the composition for preventing or treating colitis according to another embodiment of the present invention may be obtained from the Galla Rhois extract, the Anemarrhena asphodeloides Bunge extract, and the Codonopsis lanceolata extract, respectively. At this time, the alcohol used for obtaining the soluble fractions has 3 to 8 carbon atoms, and in consideration of the solubility of the active substances of the Galla Rhois extract, the Anemarrhena asphodeloides Bunge extract, and the Codonopsis lanceolata extract and a decompressed concentration process after the solubilization of the active substances thereof, it may have preferably 3 to 6 carbon atoms, more preferably 3 to 5 carbon atoms, and still more preferably 4 carbon atoms. In the present invention, the alcohol having 4 carbon atoms, which is used for the solubilizations of the Galla Rhois extract, the Anemarrhena asphodeloides Bunge extract, and the Codonopsis lanceolata extract, may be n-butanol, sec-butanol, isobutanol, tert-butanol, and the like. In the present invention, the alcohol-soluble fraction of the Galla Rhois extract, the alcohol-soluble fraction of the Anemarrhena asphodeloides Bunge extract, and the alcohol-soluble fraction of the Codonopsis lanceolata extract that are used as an active ingredient in the composition for preventing or treating colitis according to another embodiment of the present invention are composed of the components capable of being soluble in the alcohol having 3 to 8 carbon atoms among the components included in the Galla Rhois extract, the Anemarrhena asphodeloides Bunge extract, and the Codonopsis lanceolata extract. In this case, the Galla Rhois extract, the Anemarrhena asphodeloides Bunge extract, or the Codonopsis lanceolata extract is preferably extracted with water, the alcohol having 1 to 2 carbon atoms or the mixed solvent thereof. In addition, the alcohol-soluble fraction of the Galla Rhois extract, the alcohol-soluble fraction of the Anemarrhena asphodeloides Bunge extract, or the alcohol-soluble fraction of the Codonopsis lanceolata extract may be obtained by obtaining the Galla Rhois extract, the Anemarrhena asphodeloides Bunge extract, or the Codonopsis lanceolata extract using water, alcohol having 1 to 2 carbon atoms, or the mixture thereof as an extraction solvent, suspending the extracts through adding water to the extracts, adding the alcohol (for example, butanol) 3 to 8 carbon atoms to the suspensions, and then performing the fractionations of the suspensions thus obtained.

In addition, a Galla Rhois extract or an alcohol-soluble fraction thereof includes glucose1,2,3,4,6-penta-O-galloyl-β-D-glucose as an active ingredient. In addition, an Anemarrhena asphodeloides Bunge extract or an alcohol-soluble fraction thereof includes Mangiferin or Neomangiferin as an active ingredient. In addition, a Codonopsis lanceolata extract or an alcohol-soluble fraction thereof includes Lancemaside A as an active ingredient. However, for the active ingredients included in the Galla Rhois extract or the alcohol-soluble fraction thereof, the Anemarrhena asphodeloides Bunge extract or the alcohol-soluble fraction thereof, and the Codonopsis lanceolata extract or the alcohol-soluble fraction thereof, the types or contents of the specific components may be slightly different according to the extraction methods or fractionation methods.

The composition for preventing or treating colitis according to another preferred embodiment of the present invention includes any one (hereinafter, referred to as “Anemarrhena asphodeloides Bunge extract, and the like) selected from an Anemarrhena asphodeloides Bunge extract, a fraction that is soluble in the alcohol having 3 to 8 carbon atoms among the Anemarrhena asphodeloides Bunge extract, Mangiferin, Neomangiferin, or Timosaponin A-III; and any one (hereinafter, referred to as “Coptis extract, and the like) selected from a Coptis extract, or a fraction that is soluble in the alcohol having 3 to 8 carbon atoms among the Coptis extract, as an active ingredient. The composition for preventing or treating colitis according to another preferred embodiment of the present invention includes a combined extract composed of the extract of at least two medicinal herbs, such as, the Anemarrhena asphodeloides Bunge extract and the like and the Coptis extract and the like, as an active ingredient. At this time, a mixed ratio of the Anemarrhena asphodeloides Bunge extract and the like and the Coptis extract and the like, which constitute the combined extract, is not largely limited. For example, the combined extract may include 10 to 90 wt % of the Anemarrhena asphodeloides Bunge extract and the like or 10 to 90 wt % of the Coptis extract or the Coptis extract and the like with respect to the total molecular weight of the combined extract. In addition, the molecular weight ratio of the Anemarrhena asphodeloides Bunge extract and the like and the Coptis extract and the like in the combined extract is preferably 9:1 to 1:9 and more preferably 2:8 to 8:2. The composition for preventing or treating colitis according to another preferred embodiment of the present invention may include a combined extract in varied combinations as an active ingredient. For example, the composition for preventing or treating colitis according to another preferred embodiment of the present invention may include, as an active ingredient, the Anemarrhena asphodeloides Bunge extract and the Coptis extract; an alcohol-soluble fraction of the Anemarrhena asphodeloides Bunge extract and the Coptis extract; an alcohol-soluble fraction of the Anemarrhena asphodeloides Bunge extract, and the Coptis extract; an alcohol-soluble fraction of the Anemarrhena asphodeloides Bunge extract and an alcohol-soluble fraction of the Coptis extract; Mangiferin (or Neomangiferin) and the Coptis extract; or Mangiferin (or Neomangiferin), and an alcohol-soluble fraction of the Coptis extract. The composition for preventing or treating colitis according to another preferred embodiment of the present invention may effectively prevent or treat colitis by making the appearance of the colon to be good, suppressing the contraction of the colon, and maintaining low Myeloperoxidase (MPO) activity in the animal model with acute colitis induced by 2,4,6-trinitrobenzenesulfonic acid (TNBS), the animal model with acute colitis induced by dextran sulfate sodium (DSS), the animal model with chronic colitis induced by oxazolone, or the animal model with chronic colitis induced by dextran sulfate sodium (DSS). In addition, for the composition for preventing or treating colitis according to another embodiment of the present invention, the case of including the Anemarrhena asphodeloides Bunge extract and the like and the Coptis extract and the like exhibits excellent colitis-preventing effect or excellent colitis-treating effect because of their synergy effect, as compared with the case of including only the Anemarrhena asphodeloides Bunge extract and the like as an active ingredient or the case of including the Anemarrhena asphodeloides Bunge extract and the like, the Galla Rhois extract and the like, and the Codonopsis lanceolata extract and the like as an active ingredient.

The combined extract that is an active ingredient in the composition for preventing or treating colitis according to another preferred embodiment of the present invention may be prepared in various methods. For example, the combined extract may be prepared by mixing an Anemarrhena asphodeloides Bunge and a Coptis, adding an extraction solvent thereto, and then extracting a mixed extract; or by mixing an Anemarrhena asphodeloides Bunge and a Coptis, adding an extraction solvent thereto, extracting a mixed extract, again adding the alcohol having 3 to 8 carbon atoms to the mixed extract; and then performing the fractionation thereof. In addition, the combined extract may be prepared by obtaining an Anemarrhena asphodeloides Bunge extract and a Coptis extract from an Anemarrhena asphodeloides Bunge and a Coptis, respectively, and then mixing them; or by obtaining an Anemarrhena asphodeloides Bunge extract and a Coptis extract from an Anemarrhena asphodeloides Bunge and a Coptis, respectively, again obtaining an alcohol-soluble fraction of the Anemarrhena asphodeloides Bunge extract and an alcohol-soluble fraction of the Coptis extract from the Anemarrhena asphodeloides Bunge extract and the Coptis extract, and then mixing them. A method for preparing the Coptis extract is the same as or similar to the method for preparing the Anemarrhena asphodeloides Bunge extract as described above, and thus, a detailed explanation thereof is not provided. Meanwhile, in the present invention, a type of a Coptis used for preparing the Coptis extract is not particularly limited as long as it belongs to Coptis, and for example, examples thereof include Coptis teeta, Coptis japonica, Coptis chinensis, Coptis deltoidea, Coptis omeiensis, and the like. In addition, in the present invention, the Coptis may be preferably Coptis chinensis in consideration of the amount and the composition ratio of active ingredients having colitis-preventing effect or colitis-treating effect, in which the active ingredients are included in the rhizome of the Coptis.

In addition, the alcohol-soluble fraction of the Anemarrhena asphodeloides Bunge extract and the alcohol-soluble fraction of the Coptis extract that constitute a combined extract that is an active ingredient in the composition for preventing or treating colitis according to another preferred embodiment of the present invention are obtained from the Anemarrhena asphodeloides Bunge extract and the Coptis extract, respectively. At this time, the alcohol used for obtaining the soluble fractions has 3 to 8 carbon atoms, and in consideration of the solubility of the active substances of the Anemarrhena asphodeloides Bunge extract and the Coptis extract and a decompressed concentration process after the solubilization of the active substances thereof, it may have preferably 3 to 6 carbon atoms, more preferably 3 to 5 carbon atoms, and still more preferably 4 carbon atoms. In the present invention, the alcohol having 4 carbon atoms, which is used for the solubilizations of the Anemarrhena asphodeloides Bunge extract or the Coptis extract, may be n-butanol, sec-butanol, isobutanol, tert-butanol, and the like. The alcohol-soluble fraction of the Anemarrhena asphodeloides Bunge extract or the alcohol-soluble fraction of the Coptis extract that are used as an active ingredient in the composition for preventing or treating colitis according to another preferred embodiment of the present invention is composed of the components capable of being soluble in the alcohol having 3 to 8 carbon atoms among the components included in the Anemarrhena asphodeloides Bunge extract or the Coptis extract. In this case, the Anemarrhena asphodeloides Bunge extract or the Coptis extract is preferably extracted with water, the alcohol having 1 to 2 carbon atoms, or the mixed solvent thereof.

In addition, the alcohol-soluble fraction of the Anemarrhena asphodeloides Bunge extract or the alcohol-soluble fraction of the Coptis extract may be obtained by obtaining the Anemarrhena asphodeloides Bunge extract or the Coptis extract using water, alcohol having 1 to 2 carbon atoms, or the mixture thereof as an extraction solvent, suspending the extracts through adding water to the extracts, adding the alcohol (for example, butanol) 3 to 8 carbon atoms to the suspensions, and then performing the fractionations of the suspensions thus obtained.

In addition, in the present invention, an Anemarrhena asphodeloides Bunge extract or an alcohol-soluble fraction of the Anemarrhena asphodeloides Bunge extract includes Mangiferin or Neomangiferin as an active ingredient. In addition, in the present invention, a Coptis extract or an alcohol-soluble fraction of the Coptis extract includes Berberine as an active ingredient. In addition, the Coptis extract or an alcohol-soluble fraction of the Coptis extract further includes Palmatine, Coptisine, Columbamine, Jatrorrhizine, or the like as an active substance. However, for the active substances included in the Anemarrhena asphodeloides Bunge extract or the alcohol-soluble fraction thereof and the Coptis extract or the alcohol-soluble fraction thereof, the types or contents of the specific ingredients may be slightly different according to the extraction methods or fractionation methods.

The composition for preventing or treating colitis according to the present invention may be implemented into a pharmaceutical composition, a food composition (especially, a nutraceutical composition), or feed additives according to purpose of use or use aspect. The content of an extract-based active ingredient in the composition may be adjusted in various ranges according to a specific shape, purpose of use, or use aspect of the composition.

The content of extract-based active ingredient in the pharmaceutical composition of the present invention is 0.1 to 99 wt %, preferably 0.5 to 50 wt %, and more preferably 1 to 30 wt % with respect to the total molecular weight of the composition, but the present invention is not limited thereto. The pharmaceutical composition of the present invention may further include additives, such as, a pharmaceutically acceptable carrier, excipient, or diluents in addition to the extract-based active ingredient. The carrier, excipient, and diluents that may be included in the pharmaceutical composition of the present invention may be lactose, dextrose, sucrose, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oils. In addition, the composition for preventing or treating colitis according to the present invention may further include one or more types of known active ingredients having colitis-preventing effect or colitis-treating effect, in addition to extract-based components. The pharmaceutical composition of the present invention may be formulated into a dosage form for on oral administration or a dosage form for a parenteral administration using a general method. In the case of being formulated, it may be performed using diluents or excipient, such as, a filler, an extending agent, a binding agent, a wetting agent, a disintegrating agent, and a surfactant. A solid formulation for an oral administration includes a tablet, pills, powders, granules, capsules, and the like, and such a solid formulation may be formulated by mixing extract-based components with at least one of excipients, for example, calcium carbonate, sucrose, lactose, or gelatin. In addition, in addition to a simple excipient, a lubricant, such as magnesium stearate talc may be used. A liquid formulation for an oral administration may be a suspension, liquid for internal use, an emulsion, syrups, and the like. In addition to water and liquid paraffin that are often used as simple diluents, various excipients, for example, a wetting agent, a sweetening agent, a flavoring agent, a preserving agent, and the like may be included. A formulation for a parenteral administration may include a sterilized aqueous solution, non-aqueous solvent, suspension, emulsion, freeze-dried formulation, and suppository. As a non-aqueous solvent and a suspension solvent, propylene glycol, polyethylene glycol, vegetable oil such as olive oil, injectable ester such as ethyl oleate, and the like may be used. As a base compound of suppository, Witepsol, Macrogol, Tween 61, Cacao butter, Laurin butter, glycerol gelatin, and the like may be used. Furthermore, it may be preferably formulated according to each disease or component using a proper method in the related art or the method disclosed in Remington's Pharmaceutical Science (Latest issue), Mack Publishing Company, Easton Pa. The pharmaceutical composition of the present invention may be orally or parenterally administered to a mammal including human according to the desired method. In a way of parenteral administration, there are injection ways, such as, an external use of skin, intraperitoneal injection, intrarectal injection, subcutaneous injection, intravenous injection, intramuscular injection, or intrathoracic injection. The range of the dosage of the pharmaceutical composition of the present invention varies according to the weight, age, sex, health condition, and diet of a patient, an administration period, an administration method, an excretion rate, and a severity of a disease. A general dosage per a day of the pharmaceutical composition of the present invention is not particularly limited, for example, may be preferably 0.1 to 1000 mg/kg and more preferably 1 to 500 mg/kg based on the extract-based active ingredient, and may be administered one time a day or several times a day.

The food composition of the present invention includes in a type of pills, powders, granules, infusions, tablets, capsules, or liquid, and a specific example of the food may include meats, sausages, breads, chocolates, candies, snacks, confectionaries, pizzas, ramen, other noodles, gums, dairy products including ice creams, all kinds of soups, beverages, teas, functional water, drinks, alcohol beverages, vitamin complex, and the like, and may include all the general health foods. The content of extract-based active ingredient in the food composition of the present invention may be 0.01 to 50 wt %, preferably 0.1 to 25 wt %, and more preferably 0.5 to 10 wt % with respect to the total molecular weight of the composition, but the present invention is not limited thereto. The food composition of the present invention may include various flavoring agent or natural carbohydrates as an additional ingredient, in addition to the extract-based active ingredient. In addition, the food composition of the present invention may include various nutritional supplements, vitamins, electrolytes, flavors, coloring agents, a pectic acid and a salt thereof, an alginic acid and a salt thereof, an organic acid, a protective colloid viscosity agent, a pH controlling agent, stabilizer, a preservative, glycerin, alcohol, a carbonating agent used for a carbonated drink, and the like. In addition, the food composition of the present invention may include the flesh for preparing natural fruit juices, fruit juice drinks, and vegetable drinks. These components may be used independently or in combination of them. The above-described natural carbohydrates are monosaccharide, such as, glucose and fructose, disaccharide, such as, maltose and sucrose, and polysaccharide, such as, dextrin and cyclodextrin, and sugar alcohol, such as, xylitol, sorbitol, and erythritol. As a flavoring agent, a natural flavoring agent, such as, a thaumatin and stevia extract or synthesized flavoring agent, such as, saccharin and aspartame may be used.

The feed additives of the present invention may be composed of, for example, 0.1 to 20 wt % of an extract-based ingredient, 0.001 to 0.01 wt % of lipase, 1 to 20 wt % of 3rd calcium phosphate, 0.01 to 0.1 wt % of vitamin E, 1 to 10 wt % of enzyme powder, 0.1 to 10 wt % of lactobacillus, 0.01 to 10 wt % of bacillus culture medium, and 20 to 90 wt % of glucose, but the present invention is not limited thereto. In other words, when the extract-based ingredient may be added in an effective amount, it may be used as the feed additives of the present invention, and at this time, the effective amount means the amount capable of preventing or treating colitis when poultry or livestock is continuously taken. In addition, the amount of the extract-based ingredient is preferably in the range that does not have a bad influence, which is not an addition benefit. In addition, the feed additives may further include a carrier that can be used for the poultry or livestock. The feed additives of the present invention may further include, if necessary, various nutriments, such as vitamins, amino acids, and minerals, antioxidants, antibiotics, antimicrobials, and other additives, and a shape thereof may be a proper state, such as, powders, granules, pellets, and suspensions. The feed additives of the present invention may be supplied singly or in combination with the feed to the poultry or livestock.

Hereinafter, the present invention will be described in more detail with reference to Examples. However, the following Examples are only for illustrating the technical properties of the present invention, but are not limited to the claimed range of the present invention.

I. First Experiment:Confirmations of Effects of Anemarrhena asphodeloides Bunge Extract, Fraction Thereof, and Compound Isolated Therefrom on Preventing or Treating Colitis

1. Preparations of Anemarrhena asphodeloides Bunge Extract, Fraction Thereof, and Compound Isolated Therefrom

### PREPARATION EXAMPLE 1

### Preparation of Anemarrhena asphodeloides Bunge Extract

2 l of 80% aqueous methanol solution was added to 500 g of an Anemarrhena asphodeloides Bunge, then an extraction was performed in a water bath for about 2 hours, and then, a filtration was performed. In addition, 1 k of same solvent was added to the remained residue, and then a re-extraction was performed under the same conditions and then the filtration was performed. The filtered extraction liquid was concentrated under the reduced pressure to obtain 189 g of an Anemarrhena asphodeloides Bunge extract.

### PREPARATION EXAMPLE 2

### Preparation of Butanol-Soluble Fraction From Anemarrhena asphodeloides Bunge Extract

After 189 g of the Anemarrhena asphodeloides Bunge extract obtained from Preparation Example 1 was suspended in 1.5 l of water, 1.5 l of n-butanol was added thereto, and then, the obtained solution was left while shaking to isolate n-butanol-soluble fraction layer and water-soluble fraction layer. The n-butanol-soluble fraction layer was collected and then the collected n-butanol-soluble fraction was concentrated under the reduced pressure condition to obtain 41 g of n-butanol-soluble fraction. The yield of the n-butanol-soluble fraction was 8.2% or more based on the Anemarrhena asphodeloides Bunge, and the content of Mangiferin included in the n-butanol-soluble fraction was 10% or more.

### PREPARATION EXAMPLE 3

### Isolation of Compound From Butanol-Soluble Fraction of Anemarrhena asphodeloides Bunge Extract

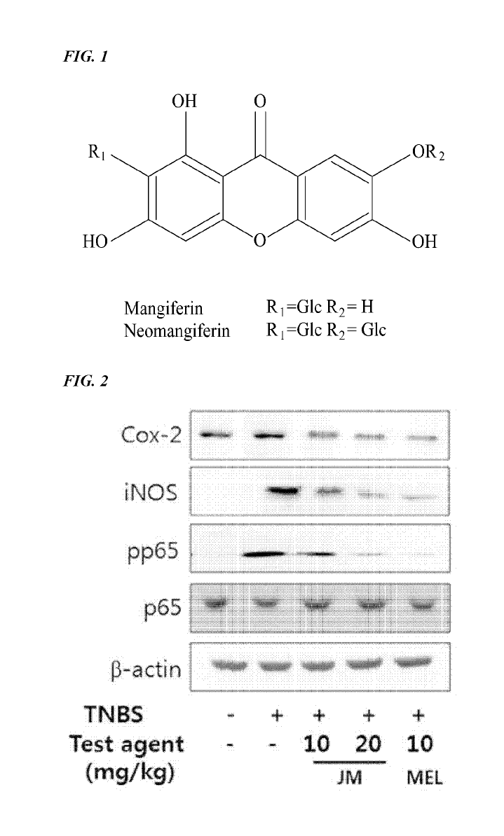

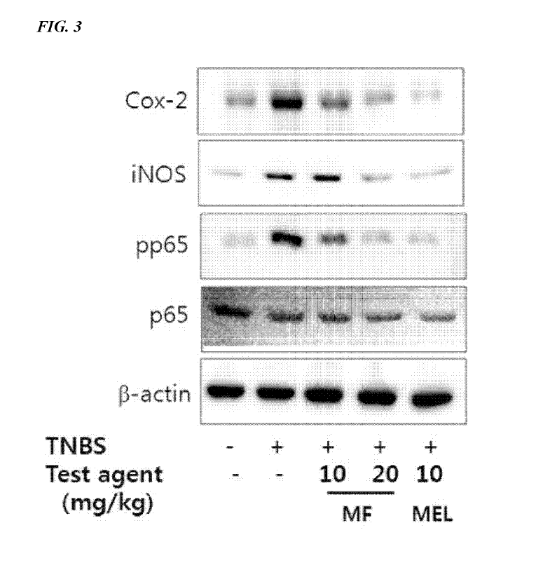

10 g of the n-butanol-soluble fraction obtained in Preparation Example 2 was subjected to a silica gel column chromatography (Merck, 10 cm×30 cm, 70 to 230 mesh) using an elution solvent (chloroform:methanol:water=65:35:10) to obtain 9 sub-fractions. Among 9 sub-fractions, Fr. VII that was a sub-fraction having the best effect at the test of animal model with colitis was subjected to a medium pressure liquid chromatography (MPLC, C18 reverse Merck, 3 cm×20 cm) using 25% methanol as an elution solvent to obtain two fractions. The two fractions were concentrated, respectively, and re-crystallized with methanol, respectively, to obtain two compounds in a type of white powder. As a result of confirming the structures of two compounds with a mass analysis and ¹³C-NMR (Bruker, AVANCE digital 400), it was confirmed that two compounds were Mangiferin and Neomangiferin, respectively. FIG. 1 illustrates Chemical Structural Formulas of Mangiferin and Neomangiferin. The yields of Mangiferin and Neomangiferin were 0.5% or more and 0.1% or more, respectively, based on the Anemarrhena asphodeloides Bunge.

<Mangiferin>

ESI(−)-MS/MS 421, 301 [M−Na]⁻

¹³C NMR (100 MHz) peaks: 162.254 (C-1), 108.04 (C-2), 164.295 (C-3), 93.813 (C-4), 103.088 (C-5), 154.606 (C-6), 144.228 (C-7), 108.489 (C-8), 179.551 (C-9), 156.697 (C-4a), 151.286 (C-4b), 112.128 (C-8a), 101.772 (C-8b), 82.025 (2-glc C-1′), 73.564 (C-2′), 71.103 (C-3′), 70.724 (C-4′), 79.449 (C-5′), 61.961 (C-6′).

<Neomangiferin>

ESI(−)-MS/MS 421, 301 [M−Na]⁻

¹³C NMR (100 MHz) 162.5 (C-1), 108.3 (C-2), 164. 5 (C-3), 94.0 (C-4), 103.3 (C-5), 156.9 (C-6), 144.4 (C-7), 112.4 (C-8), 179.8 (C-9), 154.7(C-4a), 151.5 (C-4b), 108.8 (C-8a), 102.0 (C-8b), 73.8 (2-glc C-1′), 71.3 (C-2′), 79.7 (C-3′), 71.0 (C-4′), 82.2 (C-5′), 61.4 (C-6′), 103.4 (7-glc C-1″) 73.5 (C-2″), 76.1 (C-3″), 69.6 (C-4″), 77.3 (C-5″), 60.7 (C-6″).

2. Measurement of Effect of Treating Colitis Through Test of Animal Model With Induced Colitis

(1) Preparation of Experimental Animal

4-week ICR male mice (24 to 27 g) were purchased from OrientBio. All the mice were bred under the controlled environmental conditions of 50±10% of humidity and 25±2° C. of temperature, and the lighting was repeatedly on for 12 hours and off for 12 hours. A feed for a standard experiment (Samyang, Korea) was used as a feed, and water was freely taken. For all the experiments, one group was 6 mice.

(2) Induction of Acute Colitis by TNBS and Sample Administration

Among the experimental groups, one group was set as a normal group, and for the experimental animals in other groups, colitis was induced with 2,4,6-trinitrobenzenesulfonic acid (TNBS). In detail, after lightly anesthetizing the experimental animals with ether, 0.1 ml of the solution prepared by mixing 2.5 g of 2,4,6-Trinitrobenzene sulfonic acid (NBS) solution with 50% ethanol was injected into the colon through the anus using a syringe having 1 ml volume and oval point, respectively; and then, the syringe was vertically picked up, and then, maintained for 30 seconds to induce the inflammation. Meanwhile, 0.1 ml of normal saline solution was orally administered to the normal group. Since then, from the next day, the sample dissolved in normal saline solution was orally administered in the volume that was decided in advance one time a day for 3 days. At the next day after completing the sample administration, the experimental animals were suffocated to death with carbon dioxide, the colon from appendix to the region just before the anus was removed from the colon region.

(3) Appearance Analysis of Colon and Measurement of Myeloperoxidase (MPO) Activity

1) Appearance Analysis

The scores about the removed colons were estimated by observing the appearances and the lengths of the colons according to the following criteria (Hollenbach, and the like, the criteria about the level of colon in 2005) as listed in Table 1. At this time, as a positive control group, the group administered with Mesalazine (Sigma) was used. In addition, some of the content in the colon was collected in order to analyze the rumen microorganisms, and then, was stored in a freezer of −80° C. From the tissue of colon, the content in the colon was completely removed and then the tissue of colon was washed with normal saline solution. Since then, some of the tissue was fixed with 4% formaldehyde fixing solution in order to be used as a sample for a histopathological examination, and the rest was stored in a freezer of −80° C. for a molecular biological analysis.

TABLE 1

| Macroscopic | |
|---|---|
| score | Criteria |
| 0 | There are no any kinds of ulcers and inflammations. |
| 1 | There are hyperemias without blood. |
| 2 | There are ulcers with hyperemias |
| 3 | The ulcer and inflammation were found only on one region. |
| 4 | The ulcers and inflammations were found on two or more |
| | regions. |
| 5 | The ulcer was enlarged in the size of 2 cm or more. |

2) MPO Activity Measurement

200 μl of lysis buffer was added to 100 mg of colon tissue, and then, the colon tissue was homogenized. Since then, the homogenized colon tissue was centrifuged under the conditions of 4° C. and 13000 rpm for 15 minutes, and then, the supernatant thereof was obtained. In the supernatant, the MPO activity was measured using a mouse MPO assay ELISA kit (Hbt HK210, USA). 100 μl of the supernatant was added in a 96 well plate, and then, reacted at room temperature for 1 hour. After completing the reaction, the plate was overturned and emptied; was repeatedly washed three times with 200 μl of washing buffer solution; 100 μl of diluted tracer was added thereto; and then the reaction was performed at room temperature for 1 hour. After completing the reaction, the plate was overturned and emptied; and then each well in the plate was washed using 200 μl of washing buffer solution. After repeatedly performing the washing processes three times using 200 μl of washing buffer solution, 100 μl of diluted streptavidin-peroxidase conjugate was added thereto, and then, the reaction was performed at room temperature for 1 hour. After completing the reaction, the plate was overturned and emptied, and then, each well in the plate was washed with 200 μl of washing buffer solution. After the washing processes were repeatedly performed three times using 200 μl of washing buffer solution, 100 μl of TMB substrate solution was added thereto; the plate was wrapped in an aluminum foil to block out the light; and then, the reaction was performed at room temperature for 30 minutes. Since then, 100 μl of a stop solution was added to stop the reaction, and then, the absorbance was measured at 450 nm using an ELISA reader.

3) Results of Analyzing Appearance of Colon and Measuring MPO Activity of Colon

The results of analyzing the appearance of colon and measuring myeloperoxidase (MPO) activity are listed in the following Tables 2 and 3.

TABLE 2

| | Sample | | | |
|---|---|---|---|---|
| | administration | | Macroscopic | |
| Information of TNBS treatment and | volume | Colon length | score (Mean | MPO activity |
| oral administration sample | (mg/kg) | (cm) | score) | (μUnit/mg) |
| Normal group 1 | — | 7.25 ± 0.20 | 0.4 ± 0.6 | 0.8 ± 0.1 |
| TNBS-treated group 1 | — | 3.31 ± 0.39 | 3.8 ± 0.2 | 7.0 ± 0.9 |
| TNBS-treated + Anemarrhena | 20 | 3.58 ± 0.32 | 2.2 ± 0.76 | 2.5 ± 0.6 |
| asphodeloides Bunge extract- | | | | |
| administered group | | | | |
| TNBS-treated + Anemarrhena | 10 | 4.58 ± 0.32 | 1.8 ± 0.76 | 2.4 ± 0.2 |
| asphodeloides Bunge fraction- | | | | |
| administered group | | | | |
| TNBS-treated + Anemarrhena | 20 | 5.27 ± 0.72 | 1.6 ± 0.42 | 1.8 ± 0.5 |
| asphodeloides Bunge fraction- | | | | |
| administered group | | | | |
| TNBS-treated + mesalazine- | 10 | 5.78 ± 0.54 | 1.6 ± 0.54 | 1.8 ± 0.2 |
| administered group 1 | | | | |

In the above Table 2, “the Anemarrhena asphodeloides Bunge extract” was obtained in Preparation Example 1, and “the Anemarrhena asphodeloides Bunge fraction” was an n-butanol-soluble fraction of the Anemarrhena asphodeloides Bunge extract obtained in Preparation Example 2.

TABLE 3

| | Sample | | | |
|---|---|---|---|---|
| | administration | | Macroscopic | |
| Information of TNBS treatment and | volume | Colon length | score (Mean | MPO activity |
| oral administration sample | (mg/kg) | (cm) | score) | (μUnit/mg) |
| Normal group 2 | — | 7.54 ± 0.20 | 0.4 ± 0.6 | 0.2 ± 0.1 |
| TNBS-treated group 2 | — | 4.36 ± 0.41 | 3.9 ± 0.2 | 2.3 ± 0.4 |
| TNBS-treated + mangiferin- | 10 | 5.81 ± 0.09 | 2.7 ± 0.45 | 0.5 ± 0.2 |
| administered group | | | | |
| TNBS-treated + mangiferin- | 20 | 6.98 ± 0.21 | 1.7 ± 0.27 | 0.2 ± 0.2 |
| administered group | | | | |
| TNBS-treated + neomangiferin- | 10 | 5.78 ± 0.10 | 2.8 ± 0.51 | 0.6 ± 0.2 |
| administered group | | | | |
| TNBS-treated + mesalazine- | 10 | 7.1 ± 0.46 | 1.6 ± 0.55 | 0.2 ± 0.1 |
| administered group 2 | | | | |

As can be seen in the above Tables 2 and 3, all the Anemarrhena asphodeloides Bunge extract, the fraction thereof, and the specific compound isolated therefrom exhibit excellent effect on preventing or treating colitis.

(4) Analysis whether or not the expression of inflammatory marker substances are suppressed

1) Whether or Not the Eexpressions of IL-1 beta, IL-6, and TNF-Alpha are Suppressed

250 μl of RIPA butter including protease inhibitor cocktail was added to 100 mg of the colon tissue of experimental animal, and then, the colon tissue was homogenized. Since then, the homogenized colon tissue was centrifuged under the conditions of 4° C. and 13000 rpm for 15 minutes to obtain supernatant. While the supernatant was stored at −80° C., the expression amounts of IL-1 beta, IL-6, and TNF-alpha were measured using a 96-well ELISA plate kits (Pierce Biotechology, Inc., Rockford, Ill., USA). The results of measuring IL-1 beta, IL-6, and TNF-alpha are listed in the following Tables 4 and 5.

TABLE 4

| | Sample | | | |
|---|---|---|---|---|
| | administration | IL-1 beta | | TNF-alpha |
| Information of TNBS treatment and | volume | conc. | IL-6 conc. | conc. |
| oral administration sample | (mg/kg) | (pg/mg) | (pg/mg) | (pg/mg) |
| Normal group 1 | — | 1.6 ± 3.8 | 1.1 ± 1.3 | 6.3 ± 2.7 |
| TNBS-treated 1 | — | 120.0 ± 28.7 | 17.5 ± 3.9 | 193.8 ± 43.3 |
| TNBS-treated + Anemarrhena | 20 | 52.4 ± 9.1 | 5.7 ± 4.6 | 73.8 ± 7.9 |
| asphodeloides Bunge extract- | | | | |
| administered group | | | | |
| TNBS-treated + Anemarrhena | 10 | 44.4 ± 28.7 | 4.9 ± 0.4 | 61.0 ± 16.3 |
| asphodeloides Bunge fraction- | | | | |
| administered group | | | | |
| TNBS-treated + Anemarrhena | 20 | 14.0 ± 3.5 | 2.3 ± 2.2 | 26.0 ± 11.0 |
| asphodeloides Bunge fraction- | | | | |
| administered group | | | | |
| TNBS-treated + mesalazine- | 10 | 19.8 ± 14.4 | 3.1 ± 2.3 | 26.1 ± 8.8 |
| administered 1 | | | | |

In the above Table 4, “the Anemarrhena asphodeloides Bunge extract” was obtained in Preparation Example 1, and “the Anemarrhena asphodeloides Bunge fraction” was an n-butanol-soluble fraction of the Anemarrhena asphodeloides Bunge extract obtained in Preparation Example 2.

TABLE 5

| | Sample | | | |
|---|---|---|---|---|
| | administration | IL-1 beta | | TNF-alpha |
| Information of TNBS treatment and | volume | conc. | IL-6 conc. | conc. |
| oral administration sample | (mg/kg) | (pg/mg) | (pg/mg) | (pg/mg) |
| Normal group 2 | — | 17.4 ± 2.1 | 0.9 ± 0.6 | 20.1 ± 8.9 |
| TNBS-treated 2 | — | 161.0 ± 17.0 | 18.9 ± 3.2 | 192.2 ± 42.6 |
| TNBS-treated + mangiferin- | 10 | 57.4 ± 2.1 | 5.0 ± 0.6 | 83.8 ± 10.9 |
| administered group | | | | |
| TNBS-treated + mangiferin- | 20 | 28.1 ± 12.1 | 2.2 ± 0.8 | 40.8 ± 10.9 |
| administered group | | | | |
| TNBS-treated + neomangiferin- | 10 | 62.2 ± 5.1 | 6.9 ± 1.2 | 93.6 ± 9.2 |
| administered group | | | | |
| TNBS-treated + mesalazine- | 10 | 31.1 ± 2.9 | 2.9 ± 1.2 | 52.9 ± 5.7 |
| administered group 2 | | | | |

As can be seen in the above Tables 4 and 5, in the colon tissues of the experimental animals administered with the Anemarrhena asphodeloides Bunge extract, the fraction thereof, and the specific compound isolated therefrom, the expressions of IL-1 beta, IL-6, and TNF-alpha are significantly suppressed.

2) Whether or Not the Expressions of COX-2, iNOS, p65 (NF-Kappa B), and p-p65 (phosphor-NF-Kappa B) are Suppressed.

1 ml of RIPA buffer (Gibco) was added to 0.3 g of the colon tissue of the experimental animals, and then, the homogenization was performed. Since then, the homogenized colon tissue was centrifuged under the conditions of 4° C. and 13000 rpm for 15 minutes to obtain a supernatant. While the supernatant was stored at −80° C., the expression amounts of COX-2, iNOS, p65 (NF-Kappa B), p-p65 (phosphor-NF-Kappa B), and β-actin were measured using a Western blotting method. First, 50 μg of the supernatant was subjected to an electrophoresis on SDS 10% (w/v) polyacrylamide gel for 1 hour and 30 minutes. The samples subjected to the electrophoresis were transferred on a nitrocellulose paper under the condition of 100 V and 400 mA for 1 hour and 10 minutes. The nitrocellulose paper transferred with the samples was subjected to a blocking with 5% skim milk for 30 minutes; and then, washed with PBS-Tween for 5 minutes three times; and the reaction was performed overnight with first antibody (Santa Cruz Biotechnology, USA) in a ratio of 1:100. Since then, the paper was washed for 10 minutes three times; and the reaction was performed with secondary antibody (Santa Cruz Biotechnology, USA) in a ratio of 1:1000 for 1 hour and 20 minutes. Since then, the paper was washed for 15 minutes three times; was colored with fluorescence; and then, was developed.

FIG. 2 is the results illustrating the suppressions of the expressions of inflammatory marker substances in the colons of the experimental animal group administered with an n-butanol-soluble fraction of the Anemarrhena asphodeloides Bunge extract obtained in Preparation Example 2 after inducing acute colitis by treating TNBS. In FIG. 2, “JM” refers to an n-butanol-soluble fraction of the Anemarrhena asphodeloides Bunge extract obtained in Preparation Example 2 and “MEL” refers to Mesalazine. FIG. 3 is the results illustrating the suppressions of the expressions of inflammatory marker substances in the colons of the experimental animal group administered with Mangiferin after inducing acute colitis by treating TNBS. In FIG. 3, “MF” refers to Mangiferin and “MEL” refers to Mesalazine.

As can be seen in FIGS. 2 and 3, in the colon tissues of the experimental animal groups administered with the Anemarrhena asphodeloides Bunge extract, the fraction thereof, and the specific compound isolated therefrom, the expressions of COX-2, iNOS, p65 (NF-Kappa B), and p-p65 (phosphor-NF-Kappa B) are significantly suppressed.

3. Anti-Inflammatory Effect of Specific Compound Against Inflammation of Macrophagocyte Induced by LPS or PG

4-weeks ICR male mice (20 to 28 g) were purchased from OrientBio. 2 ml of sterilized 4% thioglycolate was injected into the abdominal cavities of mice; after 96 hours, the mice were anesthetized; and then, 8 ml of the RPMI medium was again injected to the abdominal cavities of the mice. After about 5 to 10 minutes, the RPMI (including macrophagocyte) medium was collected from the abdominal cavities of the mice, and then, was centrifuged under the condition of 1000 rpm for 10 minutes to remove a supernatant. In addition, a flesh RPMI was added to a precipitated layer, and thus, the sunken cells were washed two times. Since then, a flesh medium was again added thereto, and thus, the cells were re-suspended. The number of macrophagocyte was counted using a hematocytometer. The macrophagocyte was seeded in a 24 well dish in the amount of 0.5×10⁶ per a well, and then, was treated with the test sample and the inflammation-inducing substance. Since then, a RIPA buffer (Gibco) was added to each well and then a homogenization was performed. At this time, as the test sample, Mangiferin was used, and as the inflammation-inducing substance, lipopolysaccharide (LPS) or peptidoglycan (PG) was selected. Since then, the homogenized macrophagocyte sample was centrifuged under the conditions of 4° C. and 13000 rpm for 15 minutes to obtain a supernatant. While the supernatant was stored at −80° C., the expression amounts of IRAK-1 (Interleukin-1 receptor-associated kinase 1), p-IRAK-1 (phosphor-Interleukin-1 receptor-associated kinase 1), p65 (NF-Kappa B), p-p65 (phosphor-NF-Kappa B), and β-actin were measured using a Western blotting method. First, 50 μg of the supernatant was subjected to an electrophoresis on SDS 10% (w/v) polyacrylamide gel for 1 hour and 30 minutes. The samples subjected to the electrophoresis were transferred on a nitrocellulose paper under the condition of 100 V and 400 mA for 1 hour and 10 minutes. The nitrocellulose paper transferred with the samples was subjected to a blocking with 5% skim milk for 30 minutes; and then, washed with PBS-Tween for 5 minutes three times; and the reaction was performed overnight with first antibody (Santa Cruz Biotechnology, USA) in a ratio of 1:100. Since then, the paper was washed for 10 minutes three times; and the reaction was performed with secondary antibody (Santa Cruz Biotechnology, USA) in a ratio of 1:1000 for 1 hour and 20 minutes. Since then, the paper was washed for 15 minutes three times; was colored with fluorescence; and then, was developed.

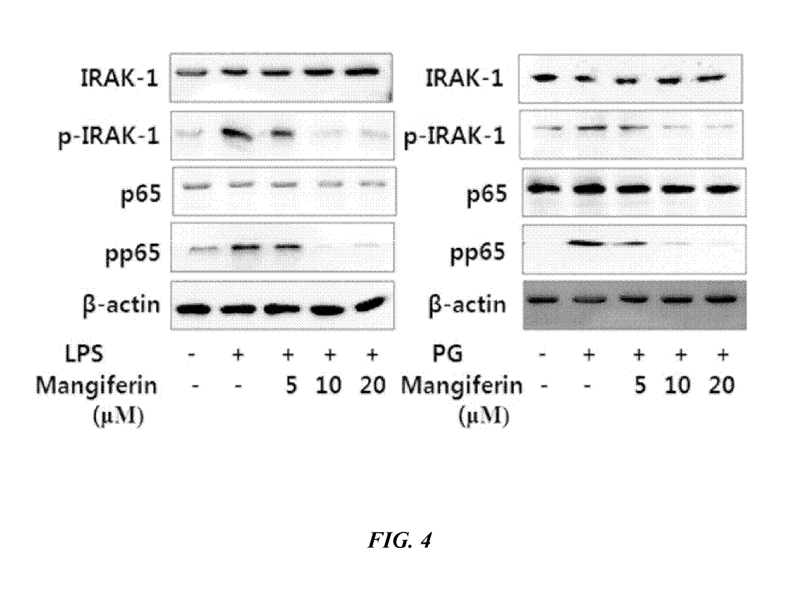

FIG. 4 is the results illustrating the suppression of the inflammatory response by Mangiferin in macrophagocyte treated with lipopolysaccharide (LPS) or peptidoglycan (PG). As can be seen in FIG. 4, Mangiferin isolated from an Anemarrhena asphodeloides Bunge exhibits excellent anti-inflammatory effect.

II. Second Experiment:Confirmation of Effect of the Combination of Anemarrhena asphodeloides Bunge Extract, Galla Rhois Extract, and Codonopsis lanceolata Extract on Preventing or Treating Colitis

4. Preparations of Galla Rhois Extract, Fraction Thereof, and Compound Isolated Therefrom

### PREPARATION EXAMPLE 4

### Preparation of Galla Rhois Extract

2 l of 80% aqueous methanol solution was added to 500 g of a Galla Rhois, then an extraction was performed in a water bath for about 2 hours, and then, a filtration was performed. In addition, 1 l of same solvent was added to the remained residue, and then a re-extraction was performed under the same conditions and then the filtration was performed. The filtered extraction liquid was concentrated under the reduced pressure to obtain 190 g of a Galla Rhois extract.

### PREPARATION EXAMPLE 5

### Preparation of Butanol-Soluble Fraction From Galla Rhois Extract

After 190 g of the Galla Rhois extract obtained from Preparation Example 4 was suspended in 1.5 1 of water, 1.5 1 of n-butanol was added thereto, and then, the obtained solution was left while shaking to isolate n-butanol-soluble fraction layer and water-soluble fraction layer. The n-butanol-soluble fraction layer was collected and then the collected n-butanol-soluble fraction was concentrated under the reduced pressure condition to obtain 102 g of n-butanol-soluble fraction. The yield of the n-butanol-soluble fraction was 21% or more based on the Galla Rhois, and the content of glucose1,2,3,4,6-penta-O-galloyl-β-D-glucose included in the n-butanol-soluble fraction was 30% or more.

### PREPARATION EXAMPLE 6

### Isolation of Compound From Butanol-Soluble Fraction of Galla Rhois Extract

20 g of the n-butanol-soluble fraction obtained in Preparation Example 5 was subjected to a silica gel column chromatography (Merck, 10 cm×50 cm, 70 to 230 mesh) using an elution solvent (chloroform:methanol:water=65:35:10) to obtain 5 sub-fractions. Among 5 sub-fractions, Fr. IV that was a sub-fraction having the best effect at the test of animal model with colitis was subjected to a re-crystallization with methanol to obtain a compound in a type of pale yellow powder. As a result of confirming the structure of the compound with ¹H-NMR (Bruker, AVANCE digital 400) and ¹³C-NMR (Bruker, AVANCE digital 400), it was confirmed that the compound was glucose1,2,3,4,6-penta-O-galloyl-β-D-glucose. FIG. 5 is Chemical Structural Formula of glucose1,2,3,4,6-penta-O-galloyl-β-D-glucose.

The yield of glucose1,2,3,4,6-penta-O-galloyl-β-D-glucose was 2.6% or more based on the Galla Rhois.

¹H-NMR (300 MHz, CD₃OD) δ: 7.12, 7.06, 7.00, 6.96 and 6.91 (each 2H, s, Gal H-2,6), 6.25 (1H, d, J=8.3 Hz, Glc H-1), 5.92 (1H, t, J=9.7 Hz, Glc H-4), 5.62 (1H, t, J=9.7 Hz, Glc H-3), 5.57 (1H, dd, J=9.7, 8.3 Hz, Glc H-2), 4.52 (1H, d, J=10.5 Hz, Glc H-6), 4.38 (1H, d, J=10.0 Hz, Glc H-5)

¹³C-NMR (500 MHz, CD₃OD) δ: 168.0. 167.4, 167.1, 167.0, 166.3, 146.7, 146.6, 146.5, 146.5, 146.3, 140.8, 140.3, 140.2, 140.1, 140.0, 139.9, 121.1, 120.4, 120.2, 120.2, 119.7, 110.6, 110.5, 110.4, 110.4, 110.3, 93.8 (Glc C-1), 74.4 (Glc C-5), 74.1 (Glc C-3), 72.2 (Glc C-2), 69.8 (Glc C-4), 63.1 (Glc C-6).

5. Preparations of Anemarrhena asphodeloides Bunge Extract, Fraction Thereof, and Compound Isolated Therefrom

### PREPARATION EXAMPLE 7

### Preparation of Anemarrhena asphodeloides Bunge Extract

2 l of 80% aqueous methanol solution was added to 500 g of an Anemarrhena asphodeloides Bunge, then an extraction was performed in a water bath for about 2 hours, and then, a filtration was performed. In addition, 1 l of same solvent was added to the remained residue, and then a re-extraction was performed under the same conditions and then the filtration was performed. The filtered extraction liquid was concentrated under the reduced pressure to obtain 189 g of an Anemarrhena asphodeloides Bunge extract.

### PREPARATION EXAMPLE 8

### Preparation of Butanol-Soluble Fraction From Anemarrhena asphodeloides Bunge Extract

After 189 g of the Anemarrhena asphodeloides Bunge extract obtained from Preparation Example 7 was suspended in 1.5 l of water, 1.5 l of n-butanol was added thereto, and then, the obtained solution was left while shaking to isolate n-butanol-soluble fraction layer and water-soluble fraction layer. The n-butanol-soluble fraction layer was collected and then the collected n-butanol-soluble fraction was concentrated under the reduced pressure condition to obtain 41 g of n-butanol-soluble fraction. The yield of the n-butanol-soluble fraction was 8.2% or more based on the Anemarrhena asphodeloides Bunge, and the content of Mangiferin included in the n-butanol-soluble fraction was 10% or more.

### PREPARATION EXAMPLE 9

### Isolation of Compound From Butanol-Soluble Fraction of Anemarrhena asphodeloides Bunge Extract

10 g of the n-butanol-soluble fraction obtained in Preparation Example 8 was subjected to a silica gel column chromatography (Merck, 10 cm×30 cm, 70 to 230 mesh) using an elution solvent (chloroform:methanol:water=65:35:10) to obtain 9 sub-fractions. Among 9 sub-fractions, Fr. VII that was a sub-fraction having the best effect at the test of animal model with colitis was subjected to a medium pressure liquid chromatography (MPLC, C18 reverse Merck, 3 cm×20 cm) using 25% methanol as an elution solvent to obtain two fractions. The two fractions were concentrated, respectively, and re-crystallized with methanol, respectively, to obtain two compounds in a type of white powder. As a result of confirming the structures of two compounds with a mass analysis and ¹³C-NMR (Bruker, AVANCE digital 400), it was confirmed that two compounds were Mangiferin and Neomangiferin, respectively. The yields of Mangiferin and Neomangiferin were 0.5% or more and 0.1% or more, respectively, based on the Anemarrhena asphodeloides Bunge.

<Mangiferin>

ESI(−)-MS/MS 421, 301 [M−Na]⁻

¹³C NMR (100 MHz) peaks:162.254 (C-1), 108.04 (C-2), 164.295 (C-3), 93.813 (C-4), 103.088 (C-5), 154.606 (C-6), 144.228 (C-7), 108.489 (C-8), 179.551 (C-9), 156.697 (C-4a), 151.286 (C-4b), 112.128 (C-8a), 101.772 (C-8b), 82.025 (2-glc C-1′), 73.564 (C-2′), 71.103 (C-3′), 70.724 (C-4′), 79.449 (C-5′), 61.961 (C-6′).

<Neomangiferin>

ESI(−)-MS/MS 421, 301 [M−Na]⁻

¹³C NMR (100 MHz) 162.5 (C-1), 108.3 (C-2), 164. 5 (C-3), 94.0 (C-4), 103.3 (C-5), 156.9 (C-6), 144.4 (C-7), 112.4 (C-8), 179.8 (C-9), 154.7 (C-4a), 151.5 (C-4b), 108.8 (C-8a), 102.0 (C-8b), 73.8 (2-glc C-1′), 71.3 (C-2′), 79.7 (C-3′), 71.0 (C-4′), 82.2 (C-5′), 61.4 (C-6′), 103.4 (7-glc C-1″) 73.5 (C-2″), 76.1 (C-3″), 69.6 (C-4″), 77.3 (C-5″), 60.7 (C-6″).

6. Preparations of Codonopsis lanceolata Extract, Fraction Thereof, and Compound Isolated Therefrom

### PREPARATION EXAMPLE 10

### Preparation of Codonopsis lanceolata Extract

4 l of 80% aqueous methanol solution was added to 2 kg of a dried Codonopsis lanceolata Trautv root (Kyoung Dong Market, Seoul, Republic Korea), then an extraction was performed in a water bath for about 2 hours, and then, a filtration was performed. In addition, 21 of same solvent was added to the remained residue, and then a re-extraction was performed under the same conditions and then the filtration was performed. The filtered extraction liquid was concentrated under the reduced pressure to obtain 185 g of a Codonopsis lanceolata extract.

### PREPARATION EXAMPLE 11

### Preparation of Butanol-Soluble Fraction From Codonopsis lanceolata Extract

After 185 g of the Codonopsis lanceolata extract obtained from Preparation Example 10 was suspended in 1.5 1 of water, 1.5 1 of n-butanol was added thereto, and then, the obtained solution was left while shaking to isolate n-butanol-soluble fraction layer and water-soluble fraction layer. The n-butanol-soluble fraction layer was collected and then the collected n-butanol-soluble fraction was concentrated under the reduced pressure condition to obtain 112 g of n-butanol-soluble fraction. The yield of the n-butanol-soluble fraction was 5.5% or more based on the Codonopsis lanceolata, and the content of Lancemaside A included in the n-butanol-soluble fraction was 4% or more.

### PREPARATION EXAMPLE 12

### Isolation of Compound from Butanol-Soluble Fraction of Codonopsis lanceolata Extract

20 g of the n-butanol-soluble fraction obtained in Preparation Example 11 was subjected to a silica gel column chromatography (Merck, 10 cm×50 cm, 70 to 230 mesh) to obtain 14 sub-fractions. At this time, a gradient elution system of dichloromethane and methanol (dichloromethane:methanol=100:0, 95:5, 90:10, 85:15, 80:20, 75:25, 70:30, 65:35, 60:40, 55:45, 50:50, 45:55, 40:60, or 30:70, in the amount of 1 1, respectively)was used as a mobile phase. In addition, among 14 sub-fractions, a 13th fraction was subjected to a medium pressure liquid chromatography (MPLC, Yamazen 540-SY-S2CSC, Japan) for an isolation for 4 hours (mobile phase:10% ACN in water to 70% ACN in water, 4 ml/min) to obtain 80 sub-fractions. Among 80 sub-fractions, 37th to 42nd fractions were concentrated under the reduced pressure and freeze-dried to obtain 620 mg of the compound in a type of white amorphous powder. As a result of confirming the structure of the compound with a mass analysis and ¹³C-NMR (Bruker, AVANCE digital 400), it was confirmed that the compound was Lancemaside A.

HR-ESI-MS (negative mode):m/z 1189.6 [M−H]⁻

¹³C-NMR (500 MHz, CD₃OD) δ: (aglycone) 38.6, 26.5, 89.0, 39.5, 55.8, 18.3, 33.4, 39.9, 47.0, 36.9, 23.7, 122.8, 144.3, 42.0, 36.1, 74.0, 49.5, 41.2, 47.1, 30.9, 35.9, 32.1, 28.1, 16.9, 15.6, 17.5, 27.1, 175.9, 33.2, 24.7, (GlcA) 107.1, 75.3, 78.2, 73.4, 77.9, 172.7, (ara) 93.4, 75.1, 69.4, 65.7, 62.7, (rha) 100.9, 71.8, 72.7, 83.3, 68.4, 18.4, (xyl) 106.0, 74.9, 87.0, 68.9, 66.8, (xyl)106.1, 75.5, 78.1, 70.8, 67.2.

7. Preparation of Kalopanax extract

### PREPARATION EXAMPLE 13

### Preparation of Kalopanax extract

5 l of 80% aqueous methanol solution was added to 3 kg of a dried Kalopanax Pictus NAKAI bark (Kyoung Dong Market, Seoul, Republic Korea), then an extraction was performed in a water bath for about 2 hours, and then, a filtration was performed. In addition, 2 l of same solvent was added to the remained residue, and then a re-extraction was performed under the same conditions and then the filtration was performed. The filtered extraction liquid was concentrated under the reduced pressure to obtain 125 g of a Kalopanax extract.

8. Preparation of Combined Extract

### PREPARATION EXAMPLE 14

50 parts by weight of the Galla Rhois extract obtained in Preparation Example 4, 25 parts by weight of the Anemarrhena asphodeloides Bunge extract obtained in Preparation Example 7, and 25 parts by weight of the Codonopsis lanceolata extract obtained in Preparation Example 10 were mixed to prepare a combined extract.

### PREPARATION EXAMPLE 15

50 parts by weight of the Galla Rhois extract obtained in Preparation Example 4, 25 parts by weight of the Codonopsis lanceolata extract obtained in Preparation Example 10, and 25 parts by weight of the Kalopanax extract obtained in Preparation Example 13 were mixed to prepare a combined extract.

### PREPARATION EXAMPLE 16

75 parts by weight of the Galla Rhois extract obtained in Preparation Example 4 and 25 parts by weight of the Kalopanax extract obtained in Preparation Example 13 were mixed to prepare a combined extract.

### PREPARATION EXAMPLE 17

50 parts by weight of the Galla Rhois extract obtained in Preparation Example 4 and 25 parts by weight of the Anemarrhena asphodeloides Bunge extract obtained in Preparation Example 7 were mixed to prepare a combined extract.

### PREPARATION EXAMPLE 18

50 parts by weight of the Codonopsis lanceolata extract obtained in Preparation Example 10 and 50 parts by weight of the Kalopanax extract obtained in Preparation Example 13 were mixed to prepare a combined extract.

### PREPARATION EXAMPLE 19

50 parts by weight of the Anemarrhena asphodeloides Bunge extract obtained in Preparation Example 7 and 50 parts by weight of the Codonopsis lanceolata extract obtained in Preparation Example 10 were mixed to prepare a combined extract.

### PREPARATION EXAMPLE 20

50 parts by weight of the butanol-soluble fraction of the Galla Rhois extract obtained in Preparation Example 5, 25 parts by weight of the butanol-soluble fraction of the Anemarrhena asphodeloides Bunge extract obtained in Preparation Example 8, and 25 parts by weight of the butanol-soluble fraction of the Codonopsis lanceolata extract obtained in Preparation Example 11 were mixed to prepare a combined extract.

9. Measurement of Effect of Treating Colitis in Test of Model Animal With Induced Colitis

(1) Preparation of Experimental Animal

4-week ICR male mice (24 to 27 g) were purchased from OrientBio. All the mice were bred under the controlled environmental conditions of 50±10% of humidity and 25±2° C. of temperature, and the lighting was repeatedly on for 12 hours and off for 12 hours. A feed for a standard experiment (Samyang, Korea) was used as a feed, and water was freely taken. For all the experiments, one group was 6 mice.

(2) Induction of Acute Colitis by TNBS and Sample Administration

Among the experimental groups, one group was set as a normal group, and for the experimental animals in other groups, colitis was induced with 2,4,6-trinitrobenzenesulfonic acid (TNBS). In detail, after lightly anesthetizing the experimental animals with ether, 0.1 ml of the solution prepared by mixing 2.5 g of 2,4,6-Trinitrobenzene sulfonic acid (NBS) solution with 50% ethanol was injected into the colon through the anus using a syringe having 1 ml volume and oval point, respectively; and then, the syringe was vertically picked up, and then, maintained for 30 seconds to induce the inflammation. Meanwhile, 0.1 ml of normal saline solution was orally administered to the normal group. Since then, from the next day, the sample dissolved in normal saline solution was orally administered in the volume that was decided in advance one time a day for 3 days. At the next day after completing the sample administration, the experimental animals were suffocated to death with carbon dioxide, and the colon from appendix to the region just before the anus was removed from the colon region. Meanwhile, as the samples to be administered, an extract-based sample and a butanol-soluble fraction-based sample were used, and the administration of the extract-based sample and the administration of the butanol-soluble fraction-based sample were separately performed.

(3) Appearance Analysis of Colon and Measurement of Myeloperoxidase (MPO) Activity

1) Appearance Analysis

The scores about the removed colons were estimated by observing the appearances and the lengths of the colons according to the following criteria (Hollenbach, and the like, the criteria about the level of colon in 2005) as listed in Table 6. At this time, as a positive control group, the group administered with Mesalazine (Sigma) was used. In addition, some of the content in the colon was collected in order to analyze the rumen microorganisms, and then, was stored in a freezer of −80° C. From the tissue of colon, the content in the colon was completely removed and then the tissue of colon was washed with normal saline solution. Since then, some of the tissue was fixed with 4% formaldehyde fixing solution in order to be used as a sample for a histopathological examination, and the rest was stored in a freezer of −80° C. for a molecular biological analysis.

TABLE 6

| Macroscopic | |
|---|---|
| score | Criteria |
| 0 | There are no any kinds of ulcers and inflammations. |
| 1 | There are hyperemias without blood. |
| 2 | There are ulcers with hyperemias |
| 3 | The ulcer and inflammation were found only on one region. |
| 4 | The ulcers and inflammations were found on two or more |
| | regions. |
| 5 | The ulcer was enlarged in the size of 2 cm or more. |

2) MPO Activity Measurement

200 μl of lysis buffer was added to 100 mg of colon tissue, and then, the colon tissue was homogenized. Since then, the homogenized colon tissue was centrifuged under the conditions of 4° C. and 13000 rpm for 15 minutes, and then, the supernatant thereof was obtained. In the supernatant, the MPO activity was measured using a mouse MPO assay ELISA kit (Hbt HK210, USA). 100 μl of the supernatant was added in a 96 well plate, and then, reacted at room temperature for 1 hour. After completing the reaction, the plate was overturned and emptied; was repeatedly washed three times with 200 μl of washing buffer solution; 100 μl of diluted tracer was added thereto; and then the reaction was performed at room temperature for 1 hour. After completing the reaction, the plate was overturned and emptied; and then each well in the plate was washed using 200 μl of washing buffer solution. After repeatedly performing the washing processes three times using 200 μl of washing buffer solution, 100 μl of diluted streptavidin-peroxidase conjugate was added thereto, and then, the reaction was performed at room temperature for 1 hour. After completing the reaction, the plate was overturned and emptied, and then, each well in the plate was washed with 200 μl of washing buffer solution. After the washing processes were repeatedly performed three times using 200 μl of washing buffer solution, 100 μl of TMB substrate solution was added thereto; the plate was wrapped in an aluminum foil to block out the light; and then, the reaction was performed at room temperature for 30 minutes. Since then, 100 μl of a stop solution was added to stop the reaction, and then, the absorbance was measured at 450 nm using an ELISA reader.

3) Results of Analyzing Appearance of Colon and Measuring MPO Activity of Colon

When administering the extract-based samples, the results of analyzing the appearance of colon and measuring myeloperoxidase (MPO) activity in the animals with acute colitis induced by TNBS are listed in the following Table 7.

TABLE 7

| | Sample | | Macroscopic | |
|---|---|---|---|---|
| Information of TNBS treatment and | administration | Colon | score (Mean | MPO activity |
| oral administration sample | volume (mg/kg) | length (cm) | score) | (μUnit/mg) |
| Normal group 1 | — | 7.26 ± 0.20 | 0.4 ± 0.55 | 0.69 ± 0.60 |
| TNBS-treated group 1 | — | 3.31 ± 0.39 | 3.8 ± 0.23 | 3.92 ± 0.55 |
| TNBS-treated + Galla Rhois extract- | 10 | 5.15 ± 0.28 | 2.2 ± 0.45 | 1.96 ± 0.08 |
| administered group | | | | |
| TNBS-treated + OJD-administered | 10 | 5.94 ± 0.75 | 1.4 ± 0.42 | 1.28 ± 0.73 |
| group | | | | |
| TNBS-treated + OHD-administered | 10 | 4.88 ± 0.22 | 2.4 ± 0.38 | 2.01 ± 0.46 |
| group | | | | |
| TNBS-treated + OH-administered | 10 | 4.78 ± 0.32 | 2.2 ± 0.27 | 2.26 ± 1.08 |
| group | | | | |
| TNBS-treated + OJ-administered | 10 | 4.50 ± 0.31 | 2.2 ± 0.76 | 2.67 ± 0.49 |
| group | | | | |
| TNBS-treated + HD-administered | 20 | 4.67 ± 0.34 | 2.2 ± 0.45 | 2.47 ± 0.02 |
| group | | | | |
| TNBS-treated + JD-administered | 20 | 5.50 ± 0.49 | 1.6 ± 0.22 | 1.58 ± 0.31 |
| group | | | | |
| TNBS-treated + mesalazine- | 10 | 5.64 ± 0.51 | 1.6 ± 0.55 | 1.42 ± 0.08 |
| administered group 1 | | | | |

In the above Table 7, “the Galla Rhois extract” was obtained in Preparation Example 4, “OJD” was the combined extract prepared in Preparation Example 14, “OHD” was the combined extract prepared in Preparation Example 15, “OH” was the combined extract prepared in Preparation Example 16, “OJ” was the combined extract prepared in Preparation Example 17, “HD” was the combined extract prepared in Preparation Example 18, and “JD” was the combined extract prepared in Preparation Example 19. As can be seen in the above Table 7, the combined extract composed of the Galla Rhois extract, the Anemarrhena asphodeloides Bunge extract, and the Codonopsis lanceolata extract exhibits the best effect of preventing or treating colitis, and also, the combined extract composed of the Codonopsis lanceolata extract and the Anemarrhena asphodeloides Bunge extract exhibits an almost similar effect as mesalazine that was used as the sample to be administered in a positive control group.

In addition, when administering the butanol-soluble fraction-based samples, the results of analyzing the appearance of removed colon and measuring myeloperoxidase (MPO) activity in the animals with acute colitis induced by TNBS are listed in the following Table 8.

TABLE 8

| | Sample | | | |
|---|---|---|---|---|
| | administration | | Macroscopic | |
| Information of TNBS treatment and | volume | Colon length | score (Mean | MPO activity |
| oral administration sample | (mg/kg) | (cm) | score) | (μUnit/mg) |
| Normal group 2 | — | 7.20 ± 0.15 | 0.4 ± 0.55 | 0.82 ± 0.14 |
| TNBS-treated group 2 | — | 3.31 ± 0.39 | 3.84 ± 0.23 | 6.99 ± 0.89 |
| TNBS-treated + OJD-administered | 10 | 5.94 ± 0.75 | 1.2 ± 0.45 | 2.09 ± 1.01 |
| group | | | | |
| TNBS-treated + Galla Rhois fraction- | 5 | 4.47 ± 0.52 | 2.7 ± 0.67 | 3.50 ± 0.94 |
| administered group | | | | |
| TNBS-treated + Galla Rhois fraction- | 10 | 5.15 ± 0.28 | 2.2 ± 0.45 | 2.88 ± 1.10 |
| administered group | | | | |
| TNBS-treated + Anemarrhena | 10 | 3.58 ± 0.32 | 2.6 ± 0.42 | 3.72 ± 2.12 |
| asphodeloides Bunge fraction- | | | | |
| administered group | | | | |
| TNBS-treated + Codonopsis | 10 | 5.09 ± 0.30 | 2.1 ± 0.22 | 3.75 ± 1.00 |
| lanceolata fraction-administered | | | | |
| group | | | | |
| TNBS-treated + mesalazine- | 10 | 5.64 ± 0.51 | 1.6 ± 0.55 | 2.98 ± 1.04 |
| administered group 2 | | | | |

In the above Table 8, “OJD” was the combined extract prepared in Preparation Example 20, “the Galla Rhois fraction” was the n-butanol-soluble fraction of the Galla Rhois extract obtained in Preparation Example 5, “the Anemarrhena asphodeloides Bunge fraction” was the n-butanol-soluble fraction of the Anemarrhena asphodeloides Bunge extract obtained in Preparation Example 8, and “the Codonopsis lanceolata fraction” was the n-butanol-soluble extract obtained in Preparation Example 11. As can be seen in the above Table 8, the combined extract composed of the butanol-soluble fraction of the Galla Rhois extract, the butanol-soluble fraction of the Anemarrhena asphodeloides Bunge extract, and the butanol-soluble fraction of the Codonopsis lanceolata extract exhibits the best effect of preventing or treating colitis, and also, the combined extract composed of the Codonopsis lanceolata extract and the Anemarrhena asphodeloides Bunge extract exhibits excellent effect on preventing or treating colitis as compared with mesalazine that was used as the sample to be administered in a positive control group.

(4) Analysis of Change Amount of Weight of Model Animal With Acute Colitis Induced by TNBS

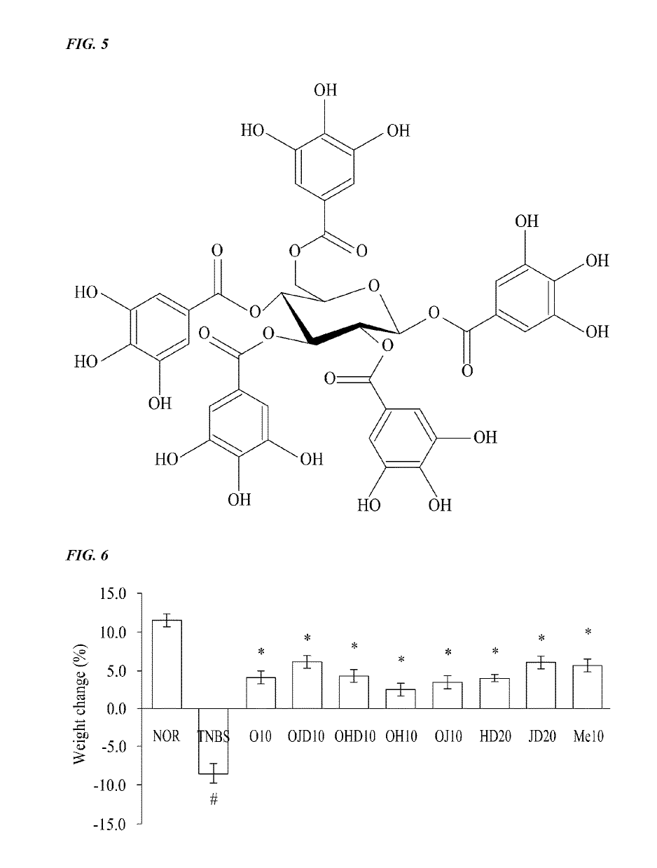

The administration of sample into the model animals with acute colitis induced by TNBS was stopped, and then, at the next day, the weights of the experimental animals were measured. The measured weights were compared with the initial weights to calculate the change amounts of weights. FIG. 6 is a graph illustrating the change amount of weight in the animal models with acute colitis induced by TNBS when an extract-based sample is administered. In FIG. 3, “NOR” of the X-axis means a normal group 1, “TNBS” means the group 1 administered only with TNBS, “010” means the group, in which 10 mg/kg of the Galla Rhois extract obtained in Preparation Example 4 was administered into the model animal with acute colitis induced by TNBS, “O10” means the group, in which 10 mg/kg of the combined extract obtained in Preparation Example 14 was administered into the model animal with acute colitis induced by TNBS, “OHD10” means the group, in which 10 mg/kg of the combined extract obtained in Preparation Example 15 was administered into the model animal with acute colitis induced by TNBS, “OH10” means the group, in which 10 mg/kg of the combined extract obtained in Preparation Example 16 was administered into the model animal with acute colitis induced by TNBS, “OJ10” means the group, in which 10 mg/kg of the combined extract obtained in Preparation Example 17 was administered into the model animal with acute colitis induced by TNBS, “HD20” means the group, in which 20 mg/kg of the combined extract obtained in Preparation Example 18 was administered into the model animal with acute colitis induced by TNBS, “JD20” means the group, in which 20 mg/kg of the combined extract obtained in Preparation Example 19 was administered into the model animal with acute colitis induced by TNBS, and “Me10” means the group 1, in which 10 mg/kg of the mesalazine was administered into the model animal with acute colitis induced by TNBS.

In addition, FIG. 7 is a graph illustrating the change amount of weight in the animal models with acute colitis induced by TNBS when a butanol-soluble fraction-based sample is administered. In FIG. 7, “NOR” of the X-axis means a normal group 2, “TNBS” means the group 2 treated only with TNBS, “OJD10” means the group, in which 10 mg/kg of the combined extract obtained in Preparation Example 20 was administered into the model animals with acute colitis induced by TNBS, “O5” means the group, in which 5 mg/kg of the butanol-soluble fraction the Galla Rhois extract obtained in Preparation Example 5 was administered into the model animals with acute colitis induced by TNBS, “010” means the group, in which 10 mg/kg of the butanol-soluble fraction the Galla Rhois extract obtained in Preparation Example 5 was administered into the model animals with acute colitis induced by TNBS, “J10” means the group, in which 10 mg/kg of the butanol-soluble fraction the Anemarrhena asphodeloides Bunge extract obtained in Preparation Example 8 was administered into the model animals with acute colitis induced by TNBS, “D10” means the group, in which 10 mg/kg of the butanol-soluble fraction the Codonopsis lanceolata extract obtained in Preparation Example 11 was administered into the model animals with acute colitis induced by TNBS, and “Me10” means the group 2, in which 10 mg/kg of mesalazine was administered into the model animals with acute colitis induced by TNBS.

As can be seen in FIGS. 6 and 7, the combined extract composed of the Galla Rhois fraction, the Anemarrhena asphodeloides Bunge extract, and the Codonopsis lanceolata extract or the combined extract composed of the butanol-soluble fraction of the Galla Rhois fraction, the butanol-soluble fraction of the Anemarrhena asphodeloides Bunge extract, and the butanol-soluble fraction of the Codonopsis lanceolata extract exhibits the almost similar amount or larger weight increasing amount as compared with mesalazine that was used as the sample to be administered in a positive control group. In addition, the combined extract composed of the Codonopsis lanceolata extract and the Anemarrhena asphodeloides Bunge extract also exhibits the almost similar weight increasing amount to mesalazine that was used as the sample to be administered in a positive control group.

(5) Analysis Whether or Not Expressions of Inflammation Marker Substances are Suppressed

1) Whether or Not the Expressions of IL-1 beta, IL-6, and TNF-Alpha are Suppressed

250 μl of RIPA butter including protease inhibitor cocktail was added to 100 mg of the colon tissue of experimental animal, and then, the colon tissue was homogenized. Since then, the homogenized colon tissue was centrifuged under the conditions of 4° C. and 13000 rpm for 15 minutes to obtain a supernatant. While the supernatant was stored at −80° C., the expression amounts of IL-1 beta, IL-6, and TNF-alpha were measured using a 96-well ELISA plate kits (Pierce Biotechology, Inc., Rockford, Ill., USA).

When the extract-based sample was administered, the results of measuring IL-1 beta, IL-6, and TNF-alpha in the colon tissues removed from the animals with acute colitis induced by TNBS are listed in the following Table 9.

TABLE 9

| | Sample | | | |
|---|---|---|---|---|
| | administration | | | |
| Information of TNBS treatment and | volume | IL-1 beta | IL-6 conc. | TNF-alpha |
| oral administration sample | (mg/kg) | conc. (pg/mg) | (pg/mg) | conc. (pg/mg) |
| Normal group 1 | — | 2.90 ± 4.57 | 0.96 ± 0.15 | 17.70 ± 6.13 |
| TNBS-treated group 1 | — | 87.25 ± 12.18 | 6.85 ± 1.47 | 85.40 ± 26.78 |
| TNBS-treated + Galla Rhois extract- | 10 | 16.81 ± 11.84 | 2.25 ± 0.28 | 31.03 ± 8.50 |
| administered group | | | | |
| TNBS-treated + OJD-administered | 10 | 9.28 ± 2.89 | 1.18 ± 0.21 | 19.43 ± 2.11 |
| group | | | | |
| TNBS-treated + OHD-administered | 10 | 29.71 ± 7.23 | 2.53 ± 0.20 | 38.62 ± 2.74 |
| group | | | | |
| TNBS-treated + OH-administered | 10 | 35.07 ± 14.13 | 2.82 ± 0.41 | 43.91 ± 13.77 |
| group | | | | |
| TNBS-treated + Of-administered | 10 | 30.22 ± 17.52 | 3.64 ± 0.61 | 59.66 ± 8.92 |
| TNBS-treated + HD-administered | 20 | 30.29 ± 14.13 | 3.11 ± 1.13 | 47.47 ± 9.02 |
| group | | | | |
| TNBS-treated + JD-administered | 20 | 21.96 ± 0.92 | 1.75 ± 0.48 | 24.14 ± 3.16 |
| group | | | | |
| TNBS-treated + mesalazine- | 10 | 16.30 ± 3.38 | 1.32 ± 0.11 | 23.79 ± 2.82 |
| administered group 1 | | | | |

In the above Table 9, “the Galla Rhois extract” was obtained in Preparation Example 4, “OJD” was the combined extract prepared in Preparation Example 14, “OHD” was the combined extract prepared in Preparation Example 15, “OH” was the combined extract prepared in Preparation Example 16, “OJ” was the combined extract prepared in Preparation Example 17, “HD” was the combined extract prepared in Preparation Example 18, and “JD” was the combined extract prepared in Preparation Example 19. As can be seen in the above Table 9, the expressions of IL-1 beta, IL-6, and TNF-alpha are significantly suppressed in the colon tissues of experimental animal groups that are administered with the combined extract composed of the Galla Rhois extract, the Anemarrhena asphodeloides Bunge extract, and the Codonopsis lanceolata extract, and the suppression level thereof are higher than that of mesalazine. In addition, the combined extract composed of the Codonopsis lanceolata extract and the Anemarrhena asphodeloides Bunge extract exhibits an almost similar effect as mesalazine that was used as the sample to be administered in a positive control group.

When administering the butanol-soluble fraction-based samples, the results of measuring the expression amounts of IL-1 beta, IL-6, and TNF-alpha in the colon tissues removed from the animals with acute colitis induced by TNBS are listed in the following Table 10.

TABLE 10

| | Sample | | | |
|---|---|---|---|---|
| | administration | | | |
| Information of TNBS treatment and | volume | IL-1 beta | IL-6 conc. | TNF-alpha |
| oral administration sample | (mg/kg) | conc. (pg/kg) | (pg/mg) | conc. (pg/mg) |
| Normal group 2 | — | 1.61 ± 3.78 | 1.14 ± 1.08 | 6.26 ± 2.73 |
| TNBS-treated group 2 | — | 120.0 ± 28.75 | 13.12 ± 2.27 | 193.84 ± 43.29 |
| TNBS-treated + OJD-administered | 10 | 17.96 ± 9.43 | 3.07 ± 1.06 | 32.02 ± 9.66 |
| group | | | | |
| TNBS-treated + Galla Rhois | 5 | 43.37 ± 27.07 | 6.60 ± 1.94 | 75.52 ± 25.60 |
| fraction-administered group | | | | |
| TNBS-treated + Galla Rhois | 10 | 30.52 ± 5.00 | 4.40 ± 2.51 | 49.29 ± 11.63 |
| fraction-administered group | | | | |
| TNBS-treated + Anemarrhena | 10 | 54.37 ± 23.27 | 6.37 ± 1.57 | 81.01 ± 24.75 |
| asphodeloides Bunge fraction- | | | | |
| administered group | | | | |
| TNBS-treated + Codonopsis | 10 | 57.70 ± 32.96 | 5.89 ± 1.49 | 91.33 ± 10.26 |
| lanceolata fraction-administered | | | | |
| group | | | | |
| TNBS-treated + mesalazine- | 10 | 31.62 ± 14.67 | 3.07 ± 1.88 | 51.04 ± 15.88 |
| administered group 2 | | | | |

In the above Table 10, “OJD” was the combined extract prepared in Preparation Example 20, “the Galla Rhois fraction” was the n-butanol-soluble fraction of the Galla Rhois extract obtained in Preparation Example 5, “the Anemarrhena asphodeloides Bunge fraction” was the n-butanol-soluble fraction of the Anemarrhena asphodeloides Bunge extract obtained in Preparation Example 8, and “the Codonopsis lanceolata fraction” was the n-butanol-soluble fraction of the Codonopsis lanceolata extract obtained in Preparation Example 11. As can be seen in the above Table 10, the expressions of IL-1 beta, IL-6, and TNF-alpha are significantly suppressed in the colon tissues that are administered with the combined extract composed of the butanol-soluble fraction of the Galla Rhois extract, the butanol-soluble fraction of the Anemarrhena asphodeloides Bunge extract, and the butanol-soluble fraction of the Codonopsis lanceolata extract.

2) Whether or Not Expressions of Inflammation Marker Substances are Suppressed

1 ml of RIPA buffer (Gibco) including protease inhibitor cocktail was added to 0.3 g of the colon tissue of the experimental animals, and then, the homogenization was performed. Since then, the homogenized colon tissue was centrifuged under the conditions of 4° C. and 13000 rpm for 15 minutes to obtain a supernatant. While the supernatant was stored at −80° C., the expression amounts of COX-2, p65 (NF-Kappa B), p-p65 (phosphor-NF-Kappa B), IRAK-1 (Interleukin-1 receptor-associated kinase 1), p-IRAK-1 (phosphor-Interleukin-1 receptor-associated kinase 1), p-IKK-P, and β-actin were measured using a Western blotting method. First, 50 μg of the supernatant was subjected to an electrophoresis on SDS 10% (w/v) polyacrylamide gel for 1 hour and 30 minutes. The samples subjected to the electrophoresis were transferred on a nitrocellulose paper under the condition of 100 V and 400 mA for 1 hour and 10 minutes. The nitrocellulose paper transferred with the samples was subjected to a blocking with 5% skim milk for 30 minutes; and then, washed with PBS-Tween for 5 minutes three times; and the reaction was performed overnight with first antibody (Santa Cruz Biotechnology, USA) in a ratio of 1:100. Since then, the paper was washed for 10 minutes three times; and the reaction was performed with secondary antibody (Santa Cruz Biotechnology, USA) in a ratio of 1:1000 for 1 hour and 20 minutes. Since then, the paper was washed for 15 minutes three times; was colored with fluorescence; and then, was developed.

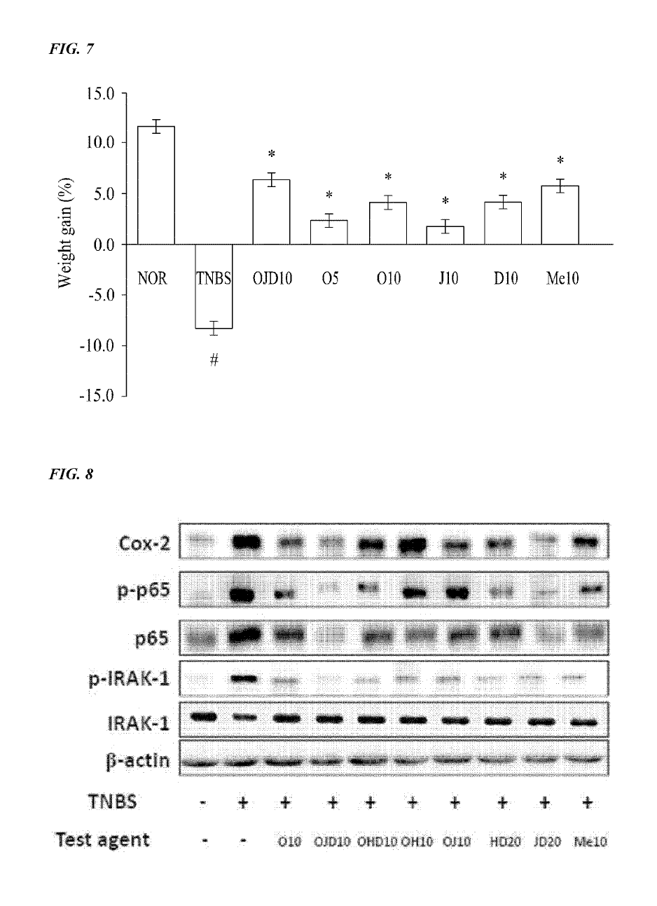

FIG. 8 is the results illustrating the suppressions of the expressions of inflammatory marker substances in the colons of the experimental animal group administered with an extract-based sample after inducing acute colitis by treating TNBS. In FIG. 8, “O10” means the group, in which 10 mg/kg of the Galla Rhois extract obtained in Preparation Example 4 was administered into the model animal with acute colitis induced by TNBS, “OJD10” means the group, in which 10 mg/kg of the combined extract obtained in Preparation Example 14 was administered into the model animal with acute colitis induced by TNBS, “OHD10” means the group, in which 10 mg/kg of the combined extract obtained in Preparation Example 15 was administered into the model animal with acute colitis induced by TNBS, “OH10” means the group, in which 10 mg/kg of the combined extract obtained in Preparation Example 16 was administered into the model animal with acute colitis induced by TNBS, “OJ10” means the group, in which 10 mg/kg of the combined extract obtained in Preparation Example 17 was administered into the model animal with acute colitis induced by TNBS, “HD20” means the group, in which 20 mg/kg of the combined extract obtained in Preparation Example 18 was administered into the model animal with acute colitis induced by TNBS, “JD20” means the group, in which 20 mg/kg of the combined extract obtained in Preparation Example 19 was administered into the model animal with acute colitis induced by TNBS, and “Me10” means the group, in which 10 mg/kg of the mesalazine was administered into the model animal with acute colitis induced by TNBS.

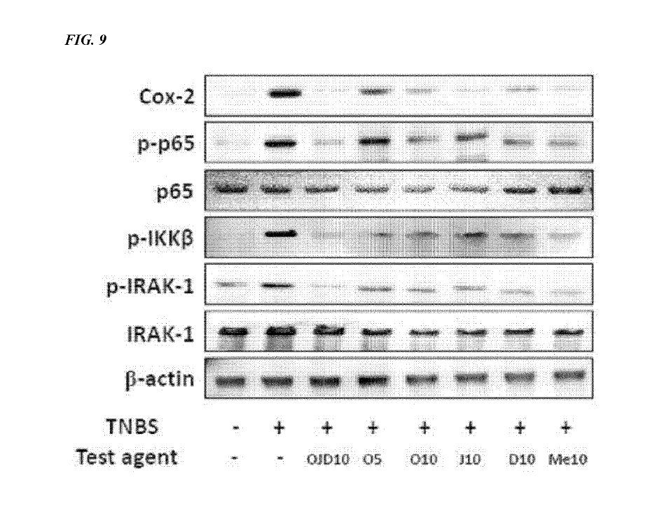

In addition, FIG. 9 is the results illustrating the suppressions of the expressions of inflammatory marker substances in the colons of the experimental animal group administered with a butanol-soluble fraction-based sample after inducing acute colitis by treating TNBS. In FIG. 9, “OJD10” means the group, in which 10 mg/kg of the combined extract obtained in Preparation Example 20 was administered into the model animals with acute colitis induced by TNBS, “O5” means the group, in which 5 mg/kg of the butanol-soluble fraction the Galla Rhois extract obtained in Preparation Example 5 was administered into the model animals with acute colitis induced by TNBS, “O10” means the group, in which 10 mg/kg of the butanol-soluble fraction the Galla Rhois extract obtained in Preparation Example 5 was administered into the model animals with acute colitis induced by TNBS, “J10” means the group, in which 10 mg/kg of the butanol-soluble fraction the Anemarrhena asphodeloides Bunge extract obtained in Preparation Example 8 was administered into the model animals with acute colitis induced by TNBS, “D10” means the group, in which 10 mg/kg of the butanol-soluble fraction the Codonopsis lanceolata extract obtained in Preparation Example 11 was administered into the model animals with acute colitis induced by TNBS, and “Me10” means the group, in which 10 mg/kg of mesalazine was administered into the model animals with acute colitis induced by TNBS.

As can be seen in FIGS. 8 and 9, the expression amounts of COX-2, p65 (NF-Kappa B), p-p65 (phosphor-NF-Kappa B), IRAK-1 (Interleukin-1 receptor-associated kinase 1), p-IRAK-1 (phosphor-Interleukin-1 receptor-associated kinase 1), p-IKK-β are significantly reduced in the colon tissues of the experimental animal groups that are administered with the combined extract composed of the Galla Rhois extract, the Anemarrhena asphodeloides Bunge extract, and the Codonopsis lanceolata extract or the experimental animal groups that are administered with the combined extract composed of the butanol-soluble fraction of the Galla Rhois extract, the butanol-soluble fraction of the Anemarrhena asphodeloides Bunge extract, and the butanol-soluble fraction of the Codonopsis lanceolata extract, and the reducing levels of the expression amounts are higher than that of mesalazine. In addition, the combined extract composed of the Codonopsis lanceolata extract and the Anemarrhena asphodeloides Bunge extract exhibits an almost similar effect as mesalazine that was used as the sample to be administered in a positive control group.

10. Anti-Inflammatory Effect of Combined Extract Against Inflammation of Macrophagocyte Induced by LPS

4-weeks ICR male mice (20 to 28 g) were purchased from OrientBio. 2 ml of sterilized 4% thioglycolate was injected into the abdominal cavities of mice; after 96 hours, the mice were anesthetized; and then, 8 ml of the RPMI medium was again injected to the abdominal cavities of the mice. After about 5 to 10 minutes, the RPMI (including macrophagocyte) medium was re-collected from the abdominal cavities of the mice, and then, was centrifuged under the condition of 1000 rpm for 10 minutes to remove a supernatant. In addition, a flesh RPMI was added to a precipitated layer, and thus, the sunken cells were washed two times. Since then, a flesh medium was again added thereto, and thus, the cells were re-suspended. The number of macrophagocyte was counted using a hematocytometer. The macrophagocyte was seeded in a 24 well dish in the amount of 0.5×10⁶ per a well, and then, was treated with the test sample and the inflammation-inducing substance for 60 minutes. Since then, a RIPA buffer (Gibco) was added to each well and then a homogenization was performed. At this time, as the test sample, the combined extract prepared in Preparation Example 14 was used, and as the inflammation-inducing substance, lipopolysaccharide (LPS) was selected. Since then, the homogenized macrophagocyte sample was centrifuged under the conditions of 4° C. and 13000 rpm for 15 minutes to obtain a supernatant. While the supernatant was stored at −80° C., the expression amounts of COX-2, iNOS, p65 (NF-Kappa B), p-p65 (phosphor-NF-Kappa B), IRAK-1 (Interleukin-1 receptor-associated kinase 1), p-IKK-β, p-IκB-α, IκKB-α, and β-actin were measured using a Western blotting method. First, 50 μg of the supernatant was subjected to an electrophoresis on SDS 10% (w/v) polyacrylamide gel for 1 hour and 30 minutes. The samples subjected to the electrophoresis were transferred on a nitrocellulose paper under the condition of 100 V and 400 mA for 1 hour and 10 minutes. The nitrocellulose paper transferred with the samples was subjected to a blocking with 5% skim milk for 30 minutes; and then, washed with PBS-Tween for 5 minutes three times; and the reaction was performed overnight with first antibody (Santa Cruz Biotechnology, USA) in a ratio of 1:100. Since then, the paper was washed for 10 minutes three times; and the reaction was performed with secondary antibody (Santa Cruz Biotechnology, USA) in a ratio of 1: 1000 for 1 hour and 20 minutes. Since then, the paper was washed for 15 minutes three times; was colored with fluorescence; and then, was developed.

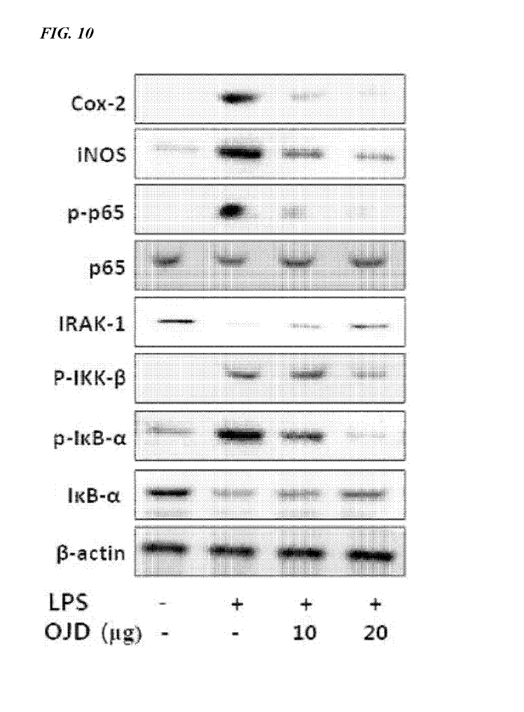

FIG. 10 is the results illustrating the suppression of inflammatory response by a combined extract composed of a Galla Rhois extract, an Anemarrhena asphodeloides Bunge extract, and a Codonopsis lanceolata extract in macrophagocyte treated with lipopolysaccharide (LPS). In FIG. 10, “OJD” means the combined extract prepared in Preparation Example 14. As can be seen in FIG. 10, the combined extract composed of the Galla Rhois extract, the Anemarrhena asphodeloides Bunge extract, and the Codonopsis lanceolata extract exhibits excellent anti-inflammatory effect.

III. Third Experiment:Confirmation of Effect of the Combination of Anemarrhena asphodeloides Bunge Extract and Coptis Extract on Preventing or Treating Colitis

11. Preparations of Anemarrhena asphodeloides Bunge Extract, Fraction Thereof, and Compound Isolated Therefrom

### PREPARATION EXAMPLE 21

### Preparation of Anemarrhena asphodeloides Bunge Extract

2 l of 80% aqueous methanol solution was added to 500 g of an Anemarrhena asphodeloides Bunge, then an extraction was performed in a water bath for about 2 hours, and then, a filtration was performed. In addition, 1 l of same solvent was added to the remained residue, and then a re-extraction was performed under the same conditions and then the filtration was performed. The filtered extraction liquid was concentrated under the reduced pressure and was freeze-dried to obtain 189 g of an Anemarrhena asphodeloides Bunge extract.

### PREPARATION EXAMPLE 22

### Preparation of Butanol-Soluble Fraction From Anemarrhena asphodeloides Bunge Extract

After 189 g of the Anemarrhena asphodeloides Bunge extract obtained from Preparation Example 21 was suspended in 1.5 l of water, 1.5 l of n-butanol was added thereto, and then, the obtained solution was left while shaking to isolate n-butanol-soluble fraction layer and water-soluble fraction layer. The n-butanol-soluble fraction layer was collected and then the collected n-butanol-soluble fraction was concentrated under the reduced pressure condition to obtain 41 g of n-butanol-soluble fraction. The yield of the n-butanol-soluble fraction was 8.2% or more based on the Anemarrhena asphodeloides Bunge, and the content of Mangiferin included in the n-butanol-soluble fraction was 10% or more.

### PREPARATION EXAMPLE 23

### Isolation of Compound From Butanol-Soluble Fraction of Anemarrhena asphodeloides Bunge Extract

10 g of the n-butanol-soluble fraction obtained in Preparation Example 22 was subjected to a silica gel column chromatography (Merck, 10 cm×30 cm, 70 to 230 mesh) using an elution solvent (chloroform:methanol:water=65:35:10) to obtain 9 sub-fractions. Among 9 sub-fractions, Fr. VII that was a sub-fraction having the best effect at the test of animal model with colitis was subjected to a medium pressure liquid chromatography (MPLC, C18 reverse Merck, 3 cm×20 cm) using 25% methanol as an elution solvent to obtain two fractions. The two fractions were concentrated, respectively, and re-crystallized with methanol, respectively, to obtain two compounds in a type of white powder. As a result of confirming the structures of two compounds with a mass analysis and ¹³C-NMR (Bruker, AVANCE digital 400), it was confirmed that two compounds were Mangiferin and Neomangiferin, respectively. FIG. 2 illustrates Chemical Structural Formulas of Mangiferin and Neomangiferin. The yields of Mangiferin and Neomangiferin were 0.5% or more and 0.1% or more, respectively, based on the Anemarrhena asphodeloides Bunge.

<Mangiferin>

ESI(−)-MS/MS 421, 301 [M−Na]⁻

¹³C NMR (100 MHz) peaks:162.254 (C-1), 108.04 (C-2), 164.295 (C-3), 93.813 (C-4), 103.088 (C-5), 154.606 (C-6), 144.228 (C-7), 108.489 (C-8), 179.551 (C-9), 156.697 (C-4a), 151.286 (C-4b), 112.128 (C-8a), 101.772 (C-8b), 82.025 (2-glc C-1′), 73.564 (C-2′), 71.103 (C-3′), 70.724 (C-4′), 79.449 (C-5′), 61.961 (C-6′).

<Neomangiferin>

ESI(−)-MS/MS 421, 301 [M−Na]⁻

¹³C NMR (100 MHz) 162.5 (C-1), 108.3 (C-2), 164. 5 (C-3), 94.0 (C-4), 103.3 (C-5), 156.9 (C-6), 144.4 (C-7), 112.4 (C-8), 179.8 (C-9), 154.7 (C-4a), 151.5 (C-4b), 108.8 (C-8a), 102.0 (C-8b), 73.8 (2-glc C-1′), 71.3 (C-2′), 79.7 (C-3′), 71.0 (C-4′), 82.2 (C-5′), 61.4 (C-6′), 103.4 (7-glc C-1″) 73.5 (C-2″), 76.1 (C-3″), 69.6 (C-4″), 77.3 (C-5″), 60.7 (C-6″).

12. Preparations of Galla Rhois Extract, Fraction Thereof, and Compound Isolated Therefrom

### PREPARATION EXAMPLE 24

### Preparation of Galla Rhois Extract

2 l of 80% aqueous methanol solution was added to 500 g of a Galla Rhois, then an extraction was performed in a water bath for about 2 hours, and then, a filtration was performed. In addition, 1 1 of same solvent was added to the remained residue, and then a re-extraction was performed under the same conditions and then the filtration was performed. The filtered extraction liquid was concentrated under the reduced pressure to obtain 190 g of a Galla Rhois extract.

### PREPARATION EXAMPLE 25

### Preparation of Butanol-Soluble Fraction From Galla Rhois Extract

After 190 g of the Galla Rhois extract obtained from Preparation Example 24 was suspended in 1.5 l of water, 1.5 l of n-butanol was added thereto, and then, the obtained solution was left while shaking to isolate n-butanol-soluble fraction layer and water-soluble fraction layer. The n-butanol-soluble fraction layer was collected and then the collected n-butanol-soluble fraction was concentrated under the reduced pressure condition and freeze-dried to obtain 102 g of n-butanol-soluble fraction. The yield of the n-butanol-soluble fraction was 21% or more based on the Galla Rhois, and the content of glucose1,2,3,4,6-penta-O-galloyl-β-D-glucose included in the n-butanol-soluble fraction was 30% or more.

13. Preparations of Codonopsis lanceolata Extract, Fraction Thereof, and Compound Isolated Therefrom

### PREPARATION EXAMPLE 26

### Preparation of Codonopsis lanceolata Extract

4 l of 80% aqueous methanol solution was added to 2 kg of a dried Codonopsis lanceolata Trautv root (Kyoung Dong Market, Seoul, Republic Korea), then an extraction was performed in a water bath for about 2 hours, and then, a filtration was performed. In addition, 21 of same solvent was added to the remained residue, and then a re-extraction was performed under the same conditions and then the filtration was performed. The filtered extraction liquid was concentrated under the reduced pressure to obtain 185 g of a Codonopsis lanceolata extract.

### PREPARATION EXAMPLE 27

### Preparation of Butanol-Soluble Fraction From Codonopsis lanceolata Extract

After 185 g of the Codonopsis lanceolata extract obtained from Preparation Example 26 was suspended in 1.5 l of water, 1.5 l of n-butanol was added thereto, and then, the obtained solution was left while shaking to isolate n-butanol-soluble fraction layer and water-soluble fraction layer. The n-butanol-soluble fraction layer was collected and then the collected n-butanol-soluble fraction was concentrated under the reduced pressure condition and freeze-dried to obtain 112 g of n-butanol-soluble fraction. The yield of the n-butanol-soluble fraction was 5.5% or more based on the Codonopsis lanceolata, and the content of Lancemaside A included in the n-butanol-soluble fraction was 4% or more.

14. Preparations of Coptis chinensis Extract and Fraction Thereof

### PREPARATION EXAMPLE 28

### Preparation of Coptis chinensis Extract

2 l of 80% aqueous ethanol solution was added to 500 g of a dried Coptis chinensis root (Kyoung Dong Market, Seoul, Republic Korea), then an extraction was performed in a water bath for about 2 hours, and then, a filtration was performed. In addition, 1 l of same solvent was added to the remained residue, and then a re-extraction was performed under the same conditions and then the filtration was performed. The filtered extraction liquid was concentrated under the reduced pressure and freeze-dried to obtain 123 g of a Coptis chinensis extract.

### PREPARATION EXAMPLE 29

### Preparation of Butanol-Soluble Fraction From Coptis chinensis Extract

After 183 g of the Coptis chinensis extract obtained from Preparation Example 28 was suspended in 1.5 l of water, 1.5 l of n-butanol was added thereto, and then, the obtained solution was left while shaking to isolate n-butanol-soluble fraction layer and water-soluble fraction layer. The n-butanol-soluble fraction layer was collected and then the collected n-butanol-soluble fraction was concentrated under the reduced pressure condition and freeze-dried to obtain 63 g of n-butanol-soluble fraction. The yield of the n-butanol-soluble fraction was 12.5% or more based on the Coptis chinensis. The ingredients in the n-butanol-soluble fraction were analyzed using a high-performance liquid chromatography (HPLC; Waters Alliance 2695 model). As a column, YMC hydrosphere C18 (S-5 μm, 120 nm, 4.6×250 mm I.D) was used, and a sample temperature, 25° C.±1 was maintained and a column temperature, 30° C.±1 was maintained. A sample concentration was prepared to be 1 mg/ml, and thus, 10 μl was injected, and the analysis was performed at a flow rate of 1.0 ml/min. In addition, as a standard substance, Berberine, Palmatine, Coptisine, and the like, which were commercialized, were purchased from Sigma, and then, used. Columbamine and Jatrorrhizine were isolated and purified from Coptis, and then, used. Using 0.2% phosphate solution (Solvent A) and methanol (Solvent B) as a mobile phase under a gradient condition, the analysis was performed for 0 minutes to 60 minutes (A:B=9:1 to 6:4), 60 minutes to 70 minutes (A:B=6:4 to 5:5), and 70 minutes to 90 minutes (A:B=5:5 to 0:10). For calculating the content of an active ingredient, an area ratio to each of standard substances was represented as a weight percentage. As the analysis results, the n-butanol-soluble fraction of Coptis chinensis extract includes about 27 to 30% of Berberine, about 7 to 8% of Palmatine, about 5 to 6% of Coptisine, about 0.8 to 1.2% of Columbamine, and about 0.8 to 1.2% of Jatrorrhizine.

15. Preparation of Combined Extract

### PREPARATION EXAMPLE 30

50 parts by weight of the butanol-soluble fraction of the Anemarrhena asphodeloides Bunge extract obtained in Preparation Example 22 and 50 parts by weight of the butanol-soluble fraction of the Galla Rhois extract obtained in Preparation Example 25 were mixed to prepare a combined extract.

### PREPARATION EXAMPLE 31

50 parts by weight of the butanol-soluble fraction of the Anemarrhena asphodeloides Bunge extract obtained in Preparation Example 22 and 50 parts by weight of the butanol-soluble fraction of the Codonopsis lanceolata extract obtained in Preparation Example 27 were mixed to prepare a combined extract.

### PREPARATION EXAMPLE 32

50 parts by weight of the butanol-soluble fraction of the Anemarrhena asphodeloides Bunge extract obtained in Preparation Example 22, 50 parts by weight of the butanol-soluble fraction of the Galla Rhois extract obtained in Preparation Example 25, and 50 parts by weight of the butanol-soluble fraction of the Codonopsis lanceolata extract obtained in Preparation Example 27 were mixed to prepare a combined extract.

### PREPARATION EXAMPLE 33

50 parts by weight of the butanol-soluble fraction of the Anemarrhena asphodeloides Bunge extract obtained in Preparation Example 22 and 50 parts by weight of the butanol-soluble fraction of the Coptis chinensis extract obtained in Preparation Example 29 were mixed to prepare a combined extract.

16. Measurement of Effect of Treating Colitis in Test of Model Animal With Acute Colitis Induced by TNBS

(1) Preparation of Experimental Animal

6-week male mice (C57BL/6, 18 to 22 g) were purchased from OrientBio. All the mice were bred under the controlled environmental conditions of 50 ±10% of humidity and 20 to 22° C. of temperature, and the lighting was repeatedly on for 12 hours and off for 12 hours. A feed for a standard experiment (Samyang, Korea) was used as a feed, and water was freely taken. For all the experiments, one group was 6 mice.

(2) Induction of Acute Colitis by TNBS and Sample Administration

Among the experimental groups, one group was set as a normal group, and for the experimental animals in other groups, acute colitis was induced with 2,4,6-trinitrobenzenesulfonic acid (TNBS). In detail, after lightly anesthetizing the experimental animals with ether, 0.1 ml of the solution prepared by mixing 2.5 g of 2,4,6-Trinitrobenzene sulfonic acid (NBS) solution with 50% ethanol was injected into the colon through the anus using a syringe having 1 ml volume and oval point, respectively; and then, the syringe was vertically picked up, and then, maintained for 30 seconds to induce the inflammation. Meanwhile, 0.1 ml of normal saline solution was orally administered to the normal group. Since then, from the next day, the sample dissolved in normal saline solution was orally administered in the volume that was decided in advance one time a day for 3 days. At the next day after completing the sample administration, the experimental animals were suffocated to death with carbon dioxide, and the colon from appendix to the region just before the anus was removed from the colon region. At this time, as a medicine sample, the experiments using the butanol-soluble fraction of the Anemarrhena asphodeloides Bunge extract, Mangiferin, and a combined extract were separately performed at a distance of time, respectively, and the effects of the medicine samples on preventing or treating colitis were reasonably analyzed based on the group supplied with normal saline solution instead of a specific medicine in the model animals with acute colitis induced by TNBS for each of the experiments.

(3) Measurements of Weight Change of Model Animal, Colon Appearance, and Myeloperoxidase (MPO) Activity

1) Analysis of Weight Change Amount

The sample administration to the model animals with colitis induced by TNBS was stopped, and at the next day, the weights of the experimental animals were measured, and then, the measured weights were compared with the initial weights to calculate the change amounts of weights.

2) Appearance Analysis

The scores about the removed colons were estimated by observing the appearances and the lengths of the colons according to the following criteria (Hollenbach, and the like, the criteria about the level of colon in 2005) as listed in Table 11. At this time, as a positive control group, the group administered with Mesalazine (Sigma) was used. In addition, some of the content in the colon was collected in order to analyze the rumen microorganisms, and then, was stored in a freezer of −80° C. From the tissue of colon, the content in the colon was completely removed and then the tissue of colon was washed with normal saline solution. Since then, some of the tissue was fixed with 4% formaldehyde fixing solution in order to be used as a sample for a histopathological examination, and the rest was stored in a freezer of −80° C. for a molecular biological analysis.

TABLE 11

| Macroscopic | |
|---|---|
| score | Criteria |
| 0 | There are no any kinds of ulcers and inflammations. |
| 1 | There are hyperemias without blood. |
| 2 | There are ulcers with hyperemias |
| 3 | The ulcer and inflammation were found only on one region. |
| 4 | The ulcers and inflammations were found on two or more |
| | regions. |
| 5 | The ulcer was enlarged in the size of 2 cm or more. |

3) Myeloperoxidase (MPO) Activity Measurement

200 μl of lysis buffer was added to 100 mg of colon tissue, and then, the colon tissue was homogenized. Since then, the homogenized colon tissue was centrifuged under the conditions of 4° C. and 13000 rpm for 15 minutes, and then, the supernatant thereof was obtained. In the supernatant, the MPO activity was measured using a mouse MPO assay ELISA kit (Hbt HK210, USA). 100 μl of the supernatant was added in a 96 well plate, and then, reacted at room temperature for 1 hour. After completing the reaction, the plate was overturned and emptied; was repeatedly washed three times with 200 μl of washing buffer solution; 100 μl of diluted tracer was added thereto; and then the reaction was performed at room temperature for 1 hour. After completing the reaction, the plate was overturned and emptied; and then each well in the plate was washed using 200 μl of washing buffer solution. After repeatedly performing the washing processes three times using 200 μl of washing buffer solution, 100 μl of diluted streptavidin-peroxidase conjugate was added thereto, and then, the reaction was performed at room temperature for 1 hour. After completing the reaction, the plate was overturned and emptied, and then, each well in the plate was washed with 200 μl of washing buffer solution. After the washing processes were repeatedly performed three times using 200 μl of washing buffer solution, 100 μl of TMB substrate solution was added thereto; the plate was wrapped in an aluminum foil to block out the light; and then, the reaction was performed at room temperature for 30 minutes. Since then, 100 μl of a stop solution was added to stop the reaction, and then, the absorbance was measured at 450 nm using an ELISA reader.

4) Results of Measuring Weight Change Amount, Appearance of Colon, Length of Colon, and MPO Activity

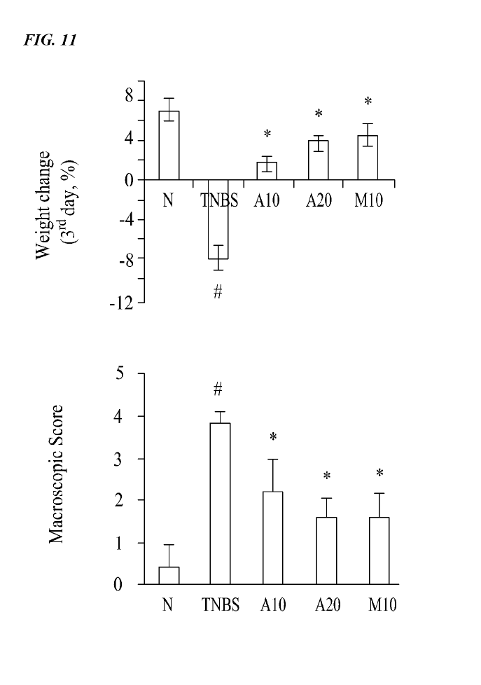

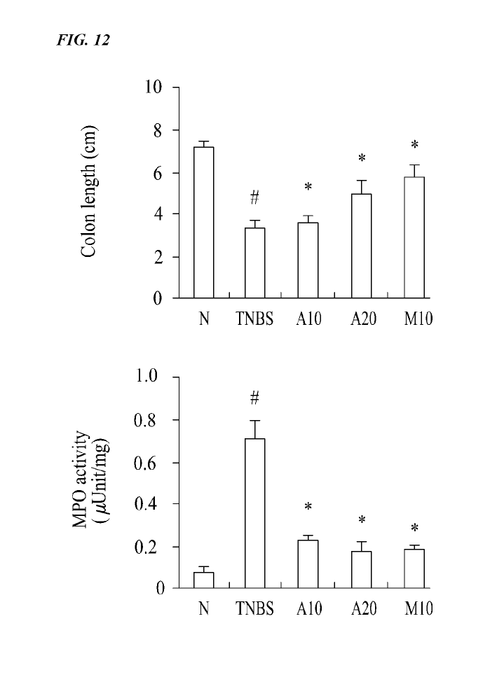

FIG. 11 is the graphs illustrating the weight changes and macroscopic scores of colons in the animal models with acute colitis induced by TNBS when an n-butanol-soluble fraction of the Anemarrhena asphodeloides Bunge extract obtained in Preparation Example 22 was used as a medicine sample and FIG. 12 is the graphs illustrating the results of the measurements of the colon lengths and MPO activity in the animal models with acute colitis induced by TNBS when an n-butanol-soluble fraction of the Anemarrhena asphodeloides Bunge extract obtained in Preparation Example 22 was used as a medicine sample. In FIGS. 11 and 12, “N” or “NOR” was the normal group, “TNBS” was the group, in which normal saline solution instead of a specific medicine sample was supplied to the model animal with acute colitis induced by TNBS, and “A” or “AJ” was the n-butanol-soluble fraction of the Anemarrhena asphodeloides Bunge extract. In addition, “A10” and “A20” were 10 mg/mouse kg and 20 mg/mouse kg as one time dosage amount of “A”, that is, a medicine sample, respectively. In addition, in FIGS. 11 and 12, “M” means mesalazine used as a positive control medicine, and one time dosage amount of mesalazine was 10 mg/mouse kg. As can be seen in FIGS. 11 and 12, the n-butanol-soluble fraction of the Anemarrhena asphodeloides Bunge extract exhibits the effect on alleviating or improving acute colitis induced by TNBS according to the concentration dependent.

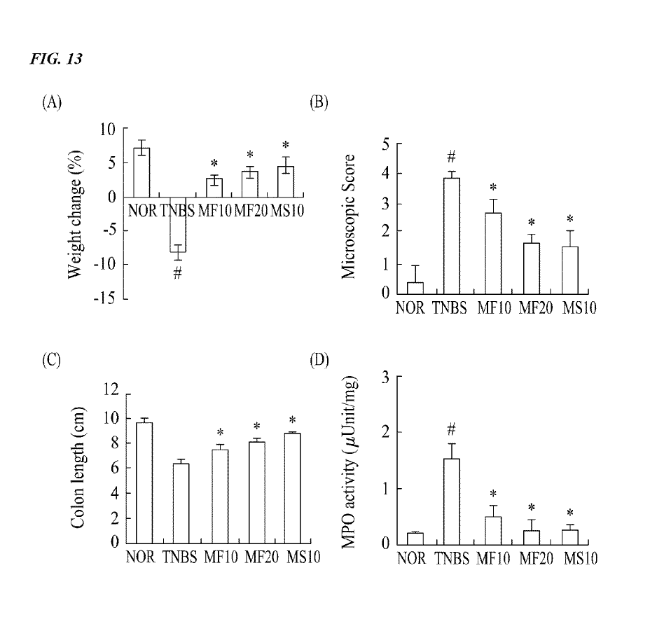

FIG. 13 is the results illustrating the results of the measurements of the weight change, the macroscopic score of colon, the colon length, and the MPO activity in the animal model with acute colitis induced by TNBS when Mangiferin obtained in Preparation Example 23 is used as a medicine sample. In FIG. 13, “NOR” represents a normal group, and “MF” represents Mangiferin. In addition, “MF10” and “MF20” respectively represent 10 mg/mouse kg and 20 mg/mouse kg as one time dosage amount of “MF” that is a medicine sample. In addition, in FIG. 13, “MS” represents mesalazine used as a positive control medicine, and one time dosage amount of mesalazine was 10 mg/mouse kg. As can be seen in FIG. 13, Mangiferin exhibits the effect on alleviating or improving acute colitis induced by TNBS according to the concentration dependent.

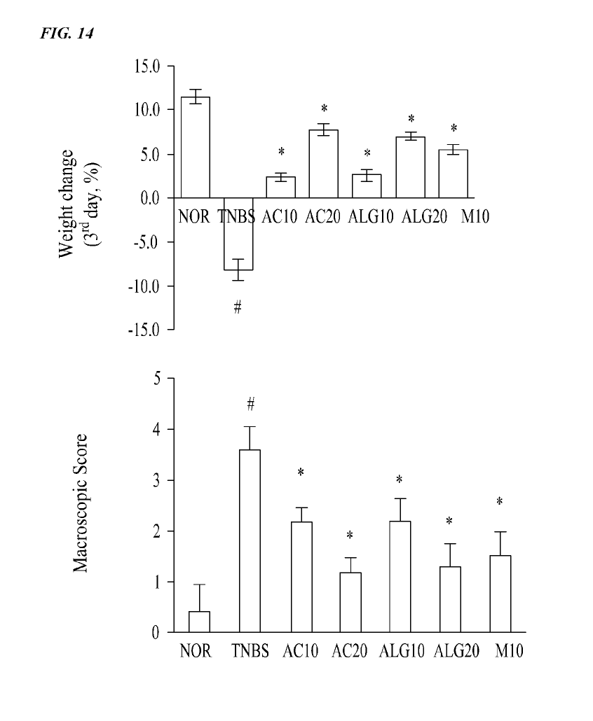

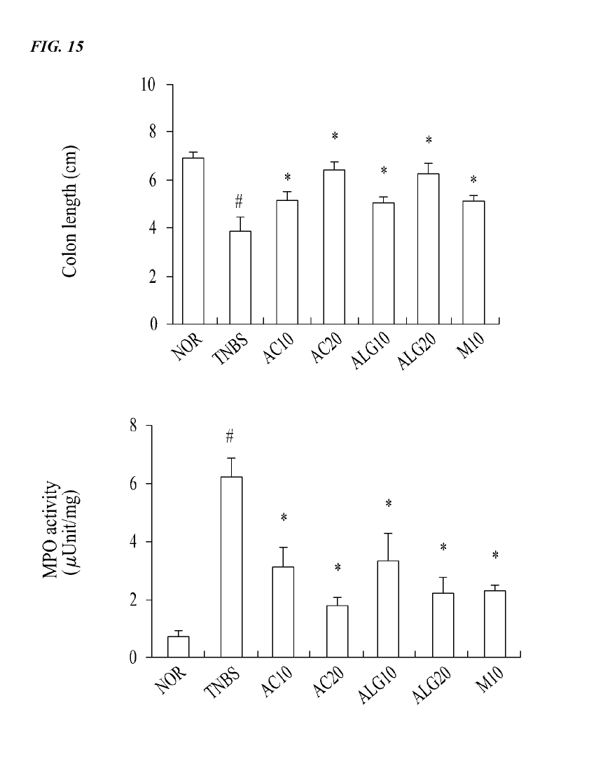

FIG. 14 is the results illustrating the weight change and the macroscopic score of colon in the animal model with acute colitis induced by TNBS when a combined extract is used as a medicine sample and FIG. 15 is the results illustrating the results of the measurements of the colon length and the MPO activity in the animal model with acute colitis induced by TNBS when a combined extract is used as a medicine sample. In FIGS. 14 and 15, “NOR” represents a normal group, “AC” represents the combined extract prepared in Preparation Example 33, and “ALG” represents the combined extract prepared in Preparation Example 32. In addition, “AC10” and “AC20” respectively represent 10 mg/mouse kg and 20 mg/mouse kg as one time dosage amount of “AC” that is a medicine sample. In addition, in FIGS. 14 and 15, “M” represents mesalazine used as a positive control medicine, and one time dosage amount of mesalazine was 10 mg/mouse kg. As can be seen in FIGS. 14 and 15, the combined extract composed of the fraction of the Anemarrhena asphodeloides Bunge extract and the fraction of the Coptis chinensis extract and the combined extract composed of the fraction of the Anemarrhena asphodeloides Bunge extract, the fraction of the Galla Rhois extract, and the fraction of the Codonopsis lanceolata extract exhibit the effect on suppressing acute colitis in the similar level as mesalazine, and especially, the combined extract of the fraction of the Anemarrhena asphodeloides Bunge extract and the fraction of the Coptis chinensis extract exhibits excellent effect as compared with the combined extract composed of the fraction of the Anemarrhena asphodeloides Bunge extract, the fraction of the Galla Rhois extract, and the fraction of the Codonopsis lanceolata extract.

(4) Analysis of Effect on Expressions of Inflammation Marker Substances

1) Effect on Expressions of Proinflammatory Cytokine and Anti-Inflammatory Cytokine

250 μl of RIPA butter including protease inhibitor cocktail was added to 100 mg of the colon tissue of experimental animal, and then, the colon tissue was homogenized. Since then, the homogenized colon tissue was centrifuged under the conditions of 4° C. and 13000 rpm for 15 minutes to obtain a supernatant. While the supernatant was stored at −80° C., the expression amounts of IL-1 beta, IL-6, and TNF-alpha, which were proinflammatory cytokines, and the expression amount of IL-10, which was anti-inflammatory cytokine, were measured using a 96-well ELISA plate kits (Pierce Biotechology, Inc., Rockford, Ill., USA).

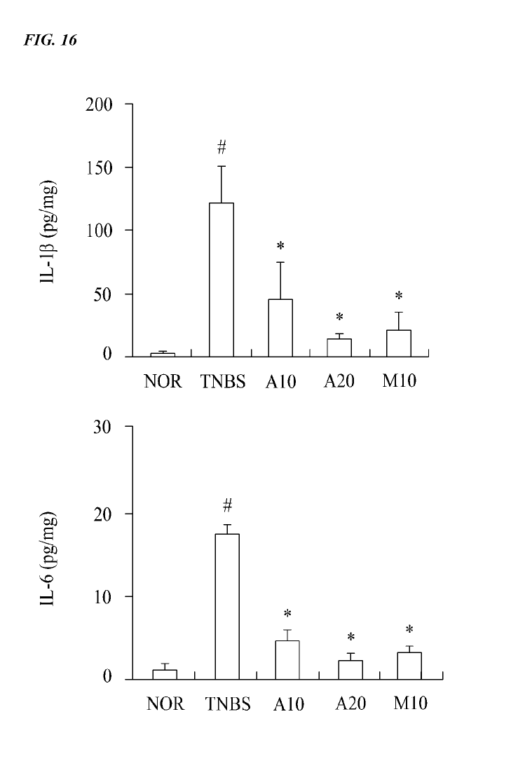

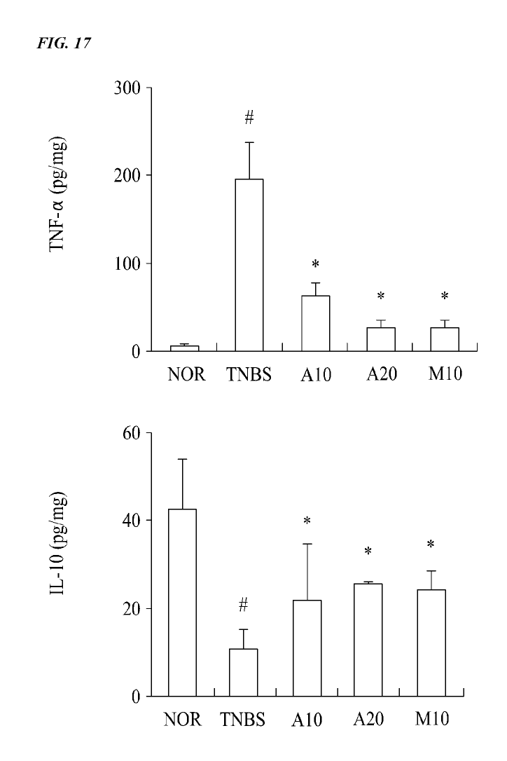

FIG. 16 is the graphs illustrating the expression amounts of IL-1 beta and IL-6 in the colon tissues of the animal model with acute colitis induced by TNBS when an n-butanol-soluble fraction of the Anemarrhena asphodeloides Bunge extract obtained in Preparation Example 22 is used as a medicine sample and FIG. 17 is the graphs illustrating the expression amounts of TNF-alpha and IL-10 in the colon tissues of the animal model with acute colitis induced by TNBS when an n-butanol-soluble fraction of the Anemarrhena asphodeloides Bunge extract obtained in Preparation Example 22 is used as a medicine sample. In FIGS. 16 and 17, “NOR” represents a normal group, and “A” represents the n-butanol-soluble fraction of the Anemarrhena asphodeloides Bunge extract. In addition, “A10” and “A20” respectively represent 10 mg/mouse kg and 20 mg/mouse kg as one time dosage amount of “A” that is a medicine sample. In addition, in FIGS. 16 and 17, “M” represents mesalazine used as a positive control medicine. As can be seen in FIGS. 16 and 17, when the n-butanol-soluble fraction of the Anemarrhena asphodeloides Bunge extract was administered, the expression amounts of IL-1 beta, IL-6, and TNF-alpha that were proinflammatory cytokines were decreased and the expression amount of IL-10 that was anti-inflammatory cytokine was increased in the colon tissues of model animals with acute colitis induced by TNBS according to the medicine administration amounts.

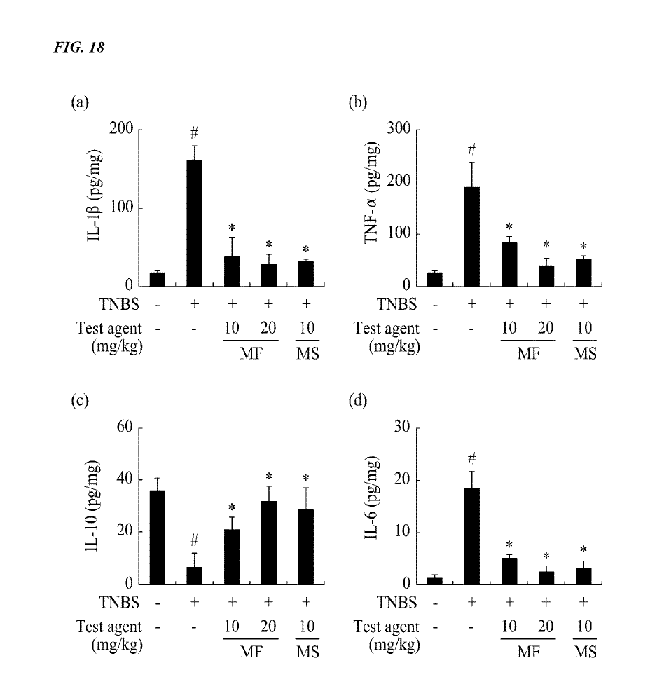

FIG. 18 is the graphs illustrating the expression amounts of proinflammatory cytokine and anti-inflammatory cytokine in the colon tissues of the animal model with acute colitis induced by TNBS when Mangiferin obtained in Preparation Example 23 is used as a medicine sample. In FIG. 18, “MF” represents Mangiferin and “MS” represents mesalazine used as a positive control medicine. As can be seen in FIG. 18, when Mangiferin was administered, the expression amounts of IL-1 beta, IL-6, and TNF-alpha that were proinflammatory cytokines were decreased and the expression amount of IL-10 that was anti-inflammatory cytokine was increased in the colon tissues of model animals with acute colitis induced by TNBS according to the medicine administration amounts.

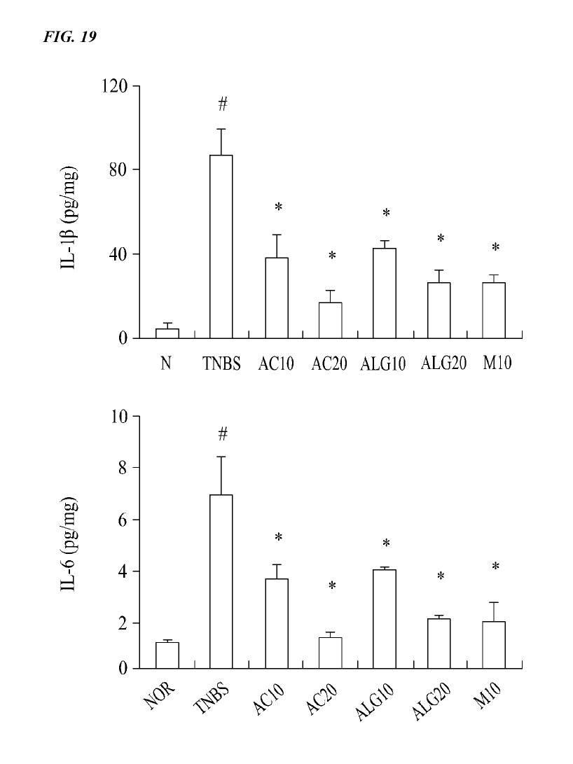

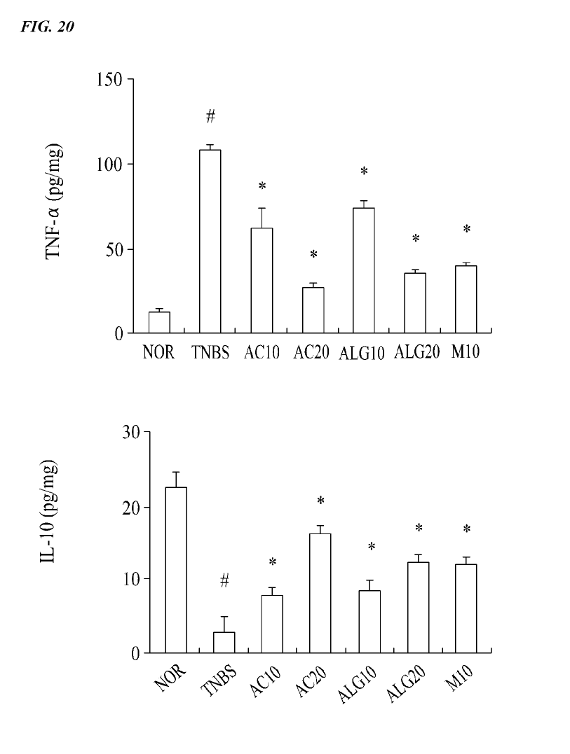

FIG. 19 is the graphs illustrating the expression amounts of IL-1 beta and IL-6 in the colon tissues of the animal model with acute colitis induced by TNBS when a combined extract is used as a medicine sample and FIG. 20 is the graphs illustrating the expression amounts of TNF-alpha and IL-10 in the colon tissues of the animal model with acute colitis induced by TNBS when a combined extract is used as a medicine sample. In FIGS. 19 and 20, “N” or “NOR” represents a normal group, “AC” represents the combined extract prepared in Preparation Example 33, and “ALG” represents the combined extract prepared in Preparation Example 32. In addition, “AC10” and “AC20” respectively represent 10 mg/mouse kg and 20 mg/mouse kg as one time dosage amount of “AC” that is a medicine sample. In addition, in FIGS. 19 and 20, “M” represents mesalazine used as a positive control medicine, and one time dosage amount of mesalazine was 10 mg/mouse kg. As can be seen in FIGS. 19 and 20, the combined extract composed of the fraction of the Anemarrhena asphodeloides Bunge extract and the fraction of the Coptis chinensis extract and the combined extract composed of the fraction of the Anemarrhena asphodeloides Bunge extract, the fraction of the Galla Rhois extract, and the fraction of the Codonopsis lanceolata extract allow the expressions of proinflammatory cytokines to be decreased and allow the expression of anti-inflammatory cytokine to be increased in the similar level as mesalazine that was used as a positive control medicine. Especially, the combined extract of the fraction of the Anemarrhena asphodeloides Bunge extract and the fraction of the Coptis chinensis extract exhibits excellent effect as compared with the combined extract composed of the fraction of the Anemarrhena asphodeloides Bunge extract, the fraction of the Galla Rhois extract, and the fraction of the Codonopsis lanceolata extract.

2) Whether or Not Expressions of Inflammation Marker Substances are Suppressed

1 ml of RIPA buffer (Gibco) including protease inhibitor cocktail was added to 0.3 g of the colon tissue of the experimental animals, and then, the homogenization was performed. Since then, the homogenized colon tissue was centrifuged under the conditions of 4° C. and 13000 rpm for 15 minutes to obtain a supernatant. While the supernatant was stored at −80° C., the expression amounts of COX-2, iNOS, p65 (NF-Kappa B), p-p65 (phosphor-NF-Kappa B), and β-actin were measured using a Western blotting method. First, 50 μg of the supernatant was subjected to an electrophoresis on SDS 10% (w/v) polyacrylamide gel for 1 hour and 30 minutes. The samples subjected to the electrophoresis were transferred on a nitrocellulose paper under the condition of 100 V and 400 mA for 1 hour and 10 minutes. The nitrocellulose paper transferred with the samples was subjected to a blocking with 5% skim milk for 30 minutes; and then, washed with PBS-Tween for 5 minutes three times; and the reaction was performed overnight with first antibody (Santa Cruz Biotechnology, USA) in a ratio of 1:100. Since then, the paper was washed for 10 minutes three times; and the reaction was performed with secondary antibody (Santa Cruz Biotechnology, USA) in a ratio of 1:1000 for 1 hour and 20 minutes. Since then, the paper was washed for 15 minutes three times; was colored with fluorescence; and then, was developed.

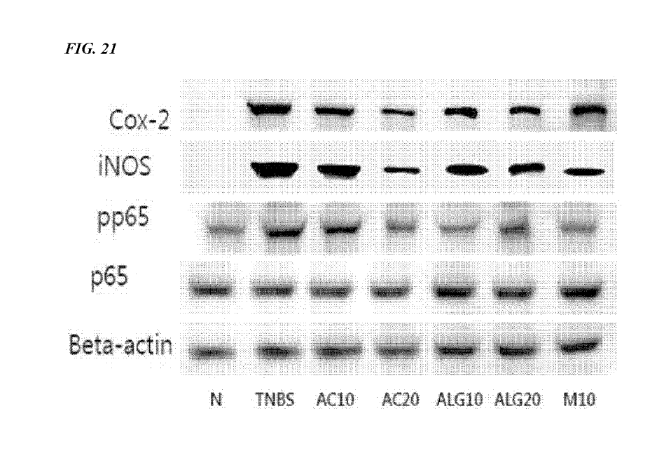

FIG. 21 is the results illustrating the suppressions of the expressions of inflammatory marker substances in the colon tissues of the animal model when a combined extract is administered to the animal model with acute colitis induced with TNBS. In FIG. 21, “N” represents a normal group, “AC” represents the combined extract prepared in Preparation Example 33, and “ALG” represents the combined extract prepared in Preparation Example 32. In addition, “AC10” and “AC20” respectively represent 10 mg/mouse kg and 20 mg/mouse kg as one time dosage amount of “AC” that is a medicine sample. In addition, in FIG. 21, “M” represents mesalazine used as a positive control medicine, and one time dosage amount of mesalazine was 10 mg/mouse kg. As can be seen in FIG. 21, the combined extract composed of the fraction of the Anemarrhena asphodeloides Bunge extract and the fraction of the Coptis chinensis extract and the combined extract composed of the fraction of the Anemarrhena asphodeloides Bunge extract, the fraction of the Galla Rhois extract, and the fraction of the Codonopsis lanceolata extract suppress the expressions of COX-2, iNOS, p-p65 (phosphor-NF-Kappa B), and the like that are proinflammatory cytokines in the similar level as mesalazine that was used as a positive control medicine. Especially, the combined extract of the fraction of the Anemarrhena asphodeloides Bunge extract and the fraction of the Coptis chinensis extract exhibits excellent effect as compared with the combined extract composed of the fraction of the Anemarrhena asphodeloides Bunge extract, the fraction of the Galla Rhois extract, and the fraction of the Codonopsis lanceolata extract.

(5) Summary of Experimental Results of Model Animal With Acute Colitis Induced by TNBS

The summary of the effects of the medicine samples administered to the model animal with acute colitis induced by TNBS on alleviating or improving colitis are listed in the following Table 12. In the following Table 12, the value of analyzed item is represented as a percentage to the value of the group supplied with normal saline solution that is used as a vehicle of a medicine sample instead of the specific medicine sample in the model animal with acute colitis induced by TNBS.

As can be seen in the following Table 12, when the combined extract composed of the fraction of the Anemarrhena asphodeloides Bunge extract and the fraction of the Coptis chinensis extract or the combined extract composed of the fraction of the Anemarrhena asphodeloides Bunge extract, the fraction of the Galla Rhois extract, and the fraction of the Codonopsis lanceolata extract is administered to the model animals with acute colitis induced by TNBS, the combined extract exhibits largely improved effect on alleviating or improving colitis as compared with the cases of administering the fraction of the Anemarrhena asphodeloides Bunge extract, the fraction of the Galla Rhois extract, the fraction of the Codonopsis lanceolata extract, or the fraction of the Coptis chinensis extract, singly, respectively. Especially, the combined extract composed of the fraction of the Anemarrhena asphodeloides Bunge extract and the fraction of the Coptis chinensis extract exhibits excellent effect as compared with the combined extract composed of the fraction of the Anemarrhena asphodeloides Bunge extract, the fraction of the Galla Rhois extract, and the fraction of the Codonopsis lanceolata extract. It is considered that this is because the fraction of the Anemarrhena asphodeloides Bunge extract and the fraction of the Coptis chinensis extract act on different targets to each other, in which the targets are related to the alleviation or treatment of colitis, and thereby exhibiting synergic effect. In addition, it can be expected that when the Anemarrhena asphodeloides Bunge extract or Mangiferin that is a compound isolated from an Anemarrhena asphodeloides Bunge is used instead of the fraction of the Anemarrhena asphodeloides Bunge extract; or the Coptis chinensis extract is used instead of the fraction of the Coptis chinensis extract, the similar effect as the fraction of the Anemarrhena asphodeloides Bunge extract and the fraction of the Coptis chinensis extract will be exhibited.

TABLE 12

| Experimental Groups | | | | | | | | | | |
|---|---|---|---|---|---|---|---|---|---|---|
| | | One time | | | | | | | | |
| | | dosage | | | | | | | | |
| Whether | | amount | Value of analyzed items (Percentage to | | | | | | | |
| or not | | of | group supplied with normal saline | | | | | | | |
| TNBS | Administered | medicine | solution after treating TNBS, %) | | | | | | | |
| was | medicine | sample | Body | Macroscopic | Colon | MPO | | IL- | | |
| treated | sample | (mg/kg) | weight | score | length | activity | TNF-α | 1β | IL-6 | IL-10 |
| − | vehicle | — | 121.8 | 11.1 | 172.5 | 4.3 | 12.1 | 4.9 | 14 | 800 |
| + | vehicle | — | 100 | 100 | 100 | 100 | 100 | 100 | 100 | 100 |
| + | Pre. Ex. 22 | 20 | 112 | 66.7 | 115 | 52.2 | 29.8 | 29.8 | 59.6 | 450 |
| + | Pre. Ex. 25 | 20 | 113.8 | 72.2 | 115 | 43.5 | 32.4 | 34.1 | 62.9 | 396.4 |
| + | Pre. Ex. 27 | 20 | 110.9 | 77.8 | 107.5 | 65.2 | 37 | 36 | 74.7 | 253.6 |
| + | Pre. Ex. 29 | 20 | 109.8 | 72.2 | 115 | 69.6 | 29.1 | 33.3 | 58.1 | 364.3 |
| + | Pre. Ex. 31 | 20 | 113.1 | 66.7 | 115 | 47.8 | 29.6 | 28.9 | 54.9 | 457.1 |
| + | Pre. Ex. 33 | 20 | 116.7 | 38.9 | 160 | 30.4 | 25 | 18.9 | 23.5 | 575 |
| + | Pre. Ex. 30 | 20 | 111.6 | 61.1 | 120 | 47.8 | 28.6 | 32.2 | 45.3 | 525 |
| + | Pre. Ex. 32 | 20 | 113.8 | 50 | 125 | 39.1 | 32.5 | 29.9 | 28.2 | 435.7 |
| + | Mesalazine | 10 | 113.5 | 61.1 | 155 | 47.8 | 36.8 | 30.3 | 26.6 | 425 |

However, when the combined extract composed of the fraction of the Anemarrhena asphodeloides Bunge extract and the fraction of the Codonopsis lanceolata extract or the combined extract composed of the fraction of the Anemarrhena asphodeloides Bunge extract and the fraction of the Galla Rhois extract is administered to the model animals with acute colitis induced by TNBS, the effect on alleviating or improving colitis is not significantly exhibited as compared with the case of administering the fraction of the Anemarrhena asphodeloides Bunge extract, the fraction of the Galla Rhois extract, the fraction of the Codonopsis lanceolata extract, or the fraction of the Coptis extract, separately.

17. Measurement of Effect of Treating Colitis in Test of Model Animal With Acute Colitis Induced by DSS

(1) Preparation of Experimental Animal

6-week male mice (C57BL/6, 18 to 22 g) were purchased from OrientBio. All the mice were bred under the controlled environmental conditions of 50±10% of humidity and 20 to 22° C. of temperature, and the lighting was repeatedly on for 12 hours and off for 12 hours. A feed for a standard experiment (Samyang, Korea) was used as a feed, and water was freely taken. For all the experiments, one group was 6 mice.

(2) Induction of Acute Colitis by DSS and Sample Administration

Among the experimental groups, one group was set as a normal group, and for the experimental animals in other groups, acute colitis was induced with dextran sulfate sodium (Molecular weight:36 to 50 kDaltons). In detail, the animal models with acute colitis were prepared by taking a 2.5% (w/v) aqueous dextran sulfate sodium solution for 7 days instead of water as drinking water. Meanwhile, the normal group was taken with water as drinking water. Since then, from the next day, the sample dissolved in normal saline solution was orally administered in the volume that was decided in advance one time a day for 3 days. At the next day after completing the sample administration, the experimental animals were suffocated to death with carbon dioxide, and the colon from appendix to the region just before the anus was removed from the colon region.

(3) Measurements of Weight Change of Model Animal, Colon Appearance, and Myeloperoxidase (MPO) Activity

1) Analysis of Weight Change Amount

The weight change amounts were analyzed in the same method as the experiment of model animal with colitis induced by TNBS.

2) Appearance Analysis

The appearance of colon was analyzed in the same method as the experiment of model animal with colitis induced by TNBS.

3) Measurement of MPO Activity

The Myeloperoxidase (MPO) activities were measured in the same method as the experiment of model animal with colitis induced by TNBS.

4) Results of Measuring Weight Change Amount, Appearance of Colon, Clon Length, and MPO Activity

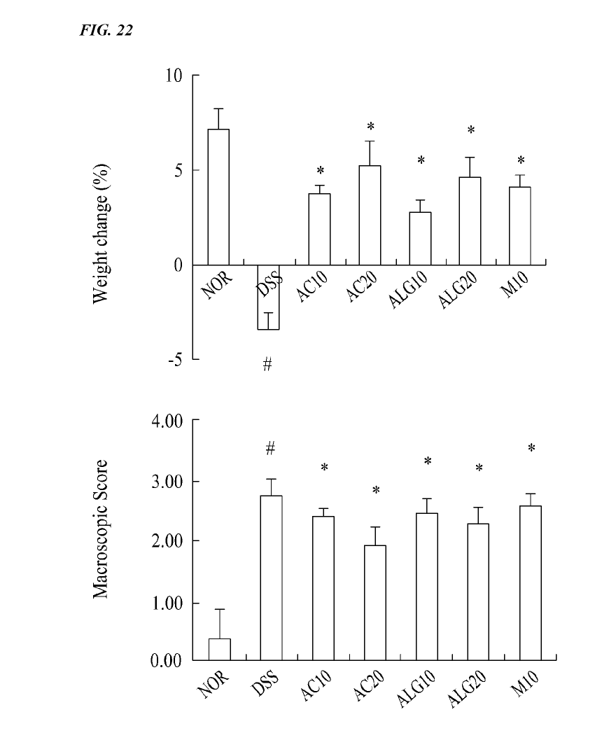

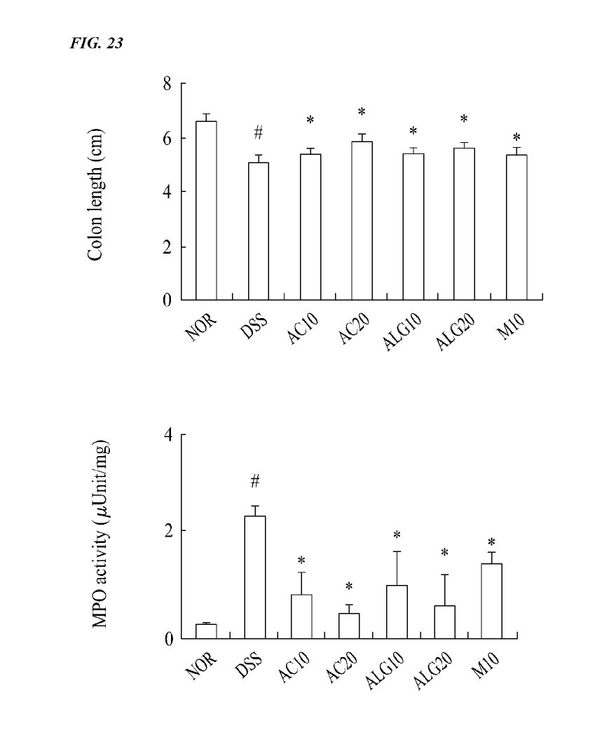

FIG. 22 is the graphs illustrating the weight change and microscopic score of the colon in the animal model with acute colitis induced by DSS when a combined extract is used as a medicine sample and FIG. 23 is the graphs illustrating the results of the measurements of the colon length and MPO activity in the animal model with acute colitis induced by DSS when a combined extract is used as a medicine sample. In FIGS. 22 and 23, “NOR” represents a normal group, “AC” represents the combined extract prepared in Preparation Example 33, and “ALG” represents the combined extract prepared in Preparation Example 32. In addition, “AC10” and “AC20” respectively represent 10 mg/mouse kg and 20 mg/mouse kg as one time dosage amount of “AC” that is a medicine sample. In addition, in FIGS. 22 and 23, “M” represents mesalazine used as a positive control medicine, and one time dosage amount of mesalazine was 10 mg/mouse kg. As can be seen in FIGS. 22 and 23, the combined extract composed of the fraction of the Anemarrhena asphodeloides Bunge extract and the fraction of the Coptis chinensis extract and the combined extract composed of the fraction of the Anemarrhena asphodeloides Bunge extract, the fraction of the Galla Rhois extract, and the fraction of the Codonopsis lanceolata extract exhibit the effect on suppressing acute colitis in the similar level as mesalazine that was used as a positive control medicine. Especially, the combined extract of the fraction of the Anemarrhena asphodeloides Bunge extract and the fraction of the Coptis chinensis extract exhibits excellent effect as compared with the combined extract composed of the fraction of the Anemarrhena asphodeloides Bunge extract, the fraction of the Galla Rhois extract, and the fraction of the Codonopsis lanceolata extract.

(4) Analysis of Effect on Expressions of Inflammation Marker Substances

1) Effect on Expressions of Proinflammatory Cytokine and Anti-Inflammatory Cytokine

When a medicine sample was administered into the model animal with acute colitis induced by DSS, the expression amounts of proinflammatory cytokine and anti-inflammatory cytokine in the colon tissue were measured in the same method as the experiment of model animal with colitis induced by TNBS.

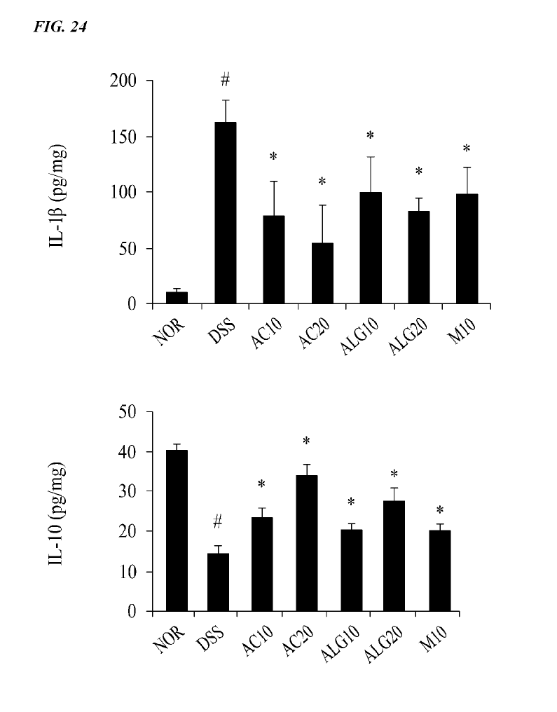

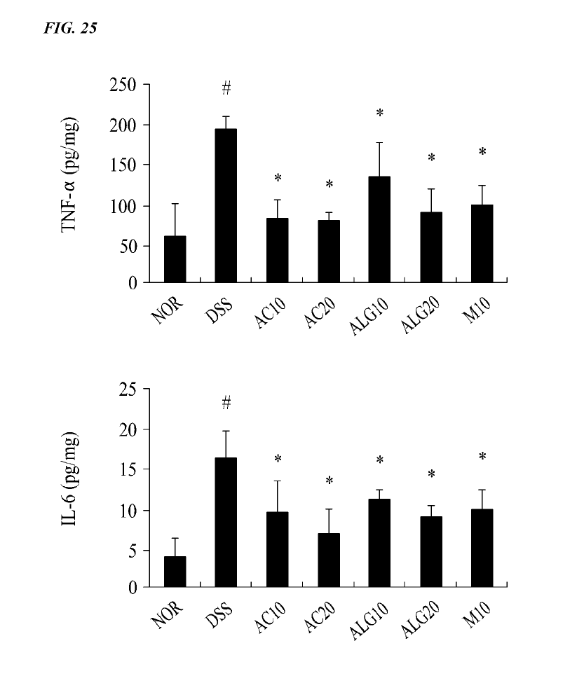

FIG. 24 is the graphs illustrating the expression amounts of IL-1 beta and IL-10 in the colon tissues of the animal model with acute colitis induced by DSS when a combined extract is used as a medicine sample and FIG. 25 is the graphs illustrating the expression amounts of TNF-alpha and IL-6 in the colon tissues of the animal model with acute colitis induced by DSS when a combined extract is used as a medicine sample. In FIGS. 24 and 25, “NOR” represents a normal group, “AC” represents the combined extract prepared in Preparation Example 33, and “ALG” represents the combined extract prepared in Preparation Example 32. In addition, “AC10” and “AC20” respectively represent 10 mg/mouse kg and 20 mg/mouse kg as one time dosage amount of “AC” that is a medicine sample. In addition, in FIGS. 24 and 25, “M” represents mesalazine used as a positive control medicine, and one time dosage amount of mesalazine was 10 mg/mouse kg. As can be seen in FIGS. 24 and 25, the combined extract composed of the fraction of the Anemarrhena asphodeloides Bunge extract and the fraction of the Coptis chinensis extract and the combined extract composed of the fraction of the Anemarrhena asphodeloides Bunge extract, the fraction of the Galla Rhois extract, and the fraction of the Codonopsis lanceolata extract allow the expressions of proinflammatory cytokines to be decreased and the expressions of anti-inflammatory cytokines to be increased in the similar level as mesalazine that was used as a positive control medicine. Especially, the combined extract of the fraction of the Anemarrhena asphodeloides Bunge extract and the fraction of the Coptis chinensis extract exhibits excellent effect as compared with the combined extract composed of the fraction of the Anemarrhena asphodeloides Bunge extract, the fraction of the Galla Rhois extract, and the fraction of the Codonopsis lanceolata extract.

2) Whether or Not Expressions of Inflammation Marker Substances are Suppressed

When a medicine sample was administered into the model animal with acute colitis induced by DSS, the expression amounts of COX-2, iNOS, p65 (NF-Kappa B), p-p65 (phosphor-NF-Kappa B), and β-actin in the colon tissue were measured in the same method as the experiment of model animal with colitis induced by TNBS.

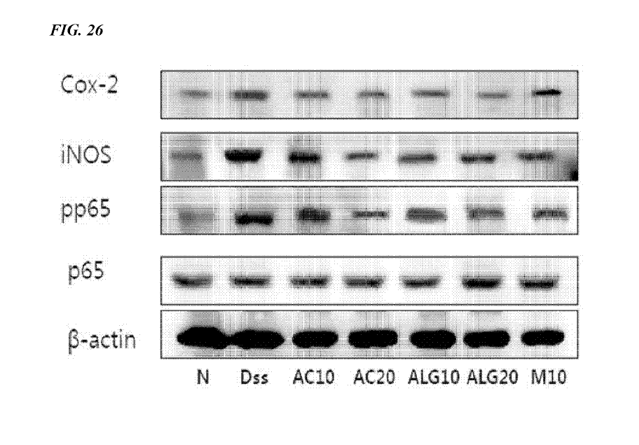

FIG. 26 is the results illustrating the suppressions of the expressions of inflammatory marker substances in the colon tissues of the animal model when a combined extract is administered to the animal model with acute colitis induced with DSS. In FIG. 26, “N” represents a normal group, “AC” represents the combined extract prepared in Preparation Example 33, and “ALG” represents the combined extract prepared in Preparation Example 32. In addition, “AC10” and “AC20” respectively represent 10 mg/mouse kg and 20 mg/mouse kg as one time dosage amount of “AC” that is a medicine sample. In addition, in FIG. 26, “M” represents mesalazine used as a positive control medicine, and one time dosage amount of mesalazine was 10 mg/mouse kg. As can be seen in FIG. 26, the combined extract composed of the fraction of the Anemarrhena asphodeloides Bunge extract and the fraction of the Coptis chinensis extract and the combined extract composed of the fraction of the Anemarrhena asphodeloides Bunge extract, the fraction of the Galla Rhois extract, and the fraction of the Codonopsis lanceolata extract allow the expressions of COX-2, iNOS, p-p65 (phosphor-NF-Kappa B), and the like to be suppressed in the similar level as mesalazine that was used as a positive control medicine. Especially, the combined extract of the fraction of the Anemarrhena asphodeloides Bunge extract and the fraction of the Coptis chinensis extract exhibits excellent effect as compared with the combined extract composed of the fraction of the Anemarrhena asphodeloides Bunge extract, the fraction of the Galla Rhois extract, and the fraction of the Codonopsis lanceolata extract.

18. Measurement of Effect of Treating Colitis in Test of Model Animal With Chronic Colitis Induced by Oxasolone

(1) Preparation of Experimental Animal

6-week male mice (C57BL/6, 18 to 22 g) were purchased from OrientBio. All the mice were bred under the controlled environmental conditions of 50±10% of humidity and 20 to 22° C. of temperature, and the lighting was repeatedly on for 12 hours and off for 12 hours. A feed for a standard experiment (Samyang, Korea) was used as a feed, and water was freely taken. For all the experiments, one group was 6 mice.

(2) Induction of Chronic Colitis by Oxazolone and Sample Administration

Among the experimental groups, one group was set as a normal group, and for the experimental animals in other groups, chronic colitis was induced with oxazolone. In detail, the hairs on the mice were removed, and 0.2 ml of 3% (w/v) oxazolone solution was applied on the area of about 1.5 cm×1.5 cm for sensitization. After 8 days, the mice were anesthetized, and then, 0.1 ml of 1% oxazolone solution was administered into the colons through rectum. Since then, from the next day, the medicine sample dissolved in normal saline solution was orally administered in the volume that was decided in advance one time a day for 15 days. At the next day after completing the sample administration, the experimental animals were suffocated to death with carbon dioxide, and the colon from appendix to the region just before the anus was removed from the colon region.

(3) Measurements of Weight Change of Model Animal, Colon Appearance, and Myeloperoxidase (MPO) Activity

1) Analysis of Weight Change Amount

The weight change amounts were analyzed in the same method as the experiment of model animal with colitis induced by TNBS.

2) Appearance Analysis

The appearance of colon was analyzed in the same method as the experiment of model animal with colitis induced by TNBS.

3) Measurement of MPO Activity

The Myeloperoxidase (MPO) activities were measured in the same method as the experiment of model animal with colitis induced by TNBS.

4) Results of Measuring Weight Change Amount, Appearance of Colon, Colon Length, and MPO Activity

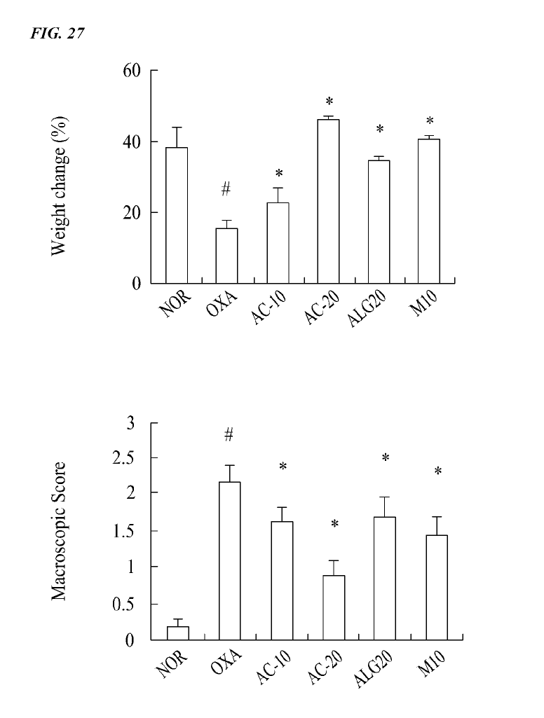

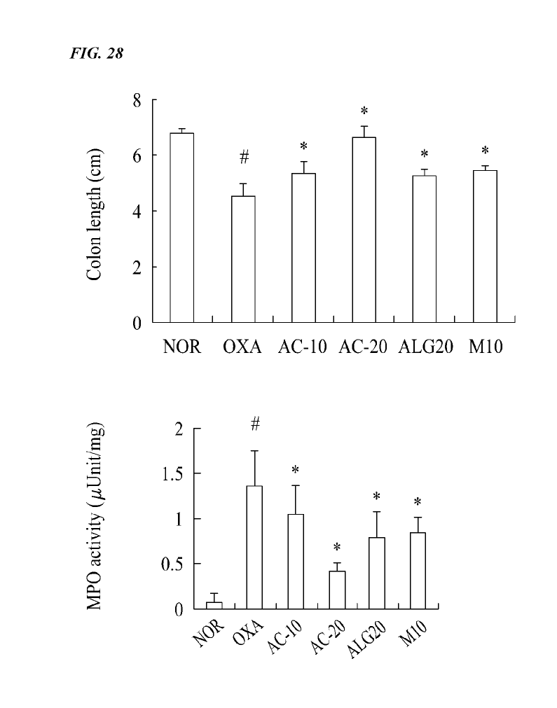

FIG. 27 is the graphs illustrating the weight change and microscopic score of the colon in the animal model with chronic colitis induced by oxazolone when a combined extract is used as a medicine sample and FIG. 28 is the graphs illustrating the results of the measurements of the colon length and MPO activity in the animal model with chronic colitis induced by oxazolone when a combined extract is used as a medicine sample. In FIGS. 27 and 28, “NOR” represents a normal group, “OXA” represents the experimental group that was orally administered only with normal saline solution after inducing chronic colitis with oxazolone, “AC” represents the combined extract prepared in Preparation Example 33, and “ALG” represents the combined extract prepared in Preparation Example 32. In addition, “AC-10” and “AC-20” respectively represent 10 mg/mouse kg and 20 mg/mouse kg as one time dosage amount of “AC” that is a medicine sample. In addition, in FIGS. 27 and 28, “M” represents mesalazine used as a positive control medicine, and one time dosage amount of mesalazine was 10 mg/mouse kg. As can be seen in FIGS. 27 and 28, the combined extract composed of the fraction of the Anemarrhena asphodeloides Bunge extract and the fraction of the Coptis chinensis extract and the combined extract composed of the fraction of the Anemarrhena asphodeloides Bunge extract, the fraction of the Galla Rhois extract, and the fraction of the Codonopsis lanceolata extract exhibit the effect on suppressing chronic colitis in the similar level as mesalazine that was used as a positive control medicine. Especially, the combined extract of the fraction of the Anemarrhena asphodeloides Bunge extract and the fraction of the Coptis chinensis extract exhibits excellent effect as compared with the combined extract composed of the fraction of the Anemarrhena asphodeloides Bunge extract, the fraction of the Galla Rhois extract, and the fraction of the Codonopsis lanceolata extract.

(4) Analysis of Effect on Expressions of Inflammation Marker Substances

1) Effect on Expressions of Proinflammatory Cytokine and Anti-Inflammatory Cytokine

When a medicine sample was administered into the model animal with chronic colitis induced by oxazolone, the expression amounts of proinflammatory cytokine and anti-inflammatory cytokine in the colon tissue were measured in the same method as the experiment of model animal with colitis induced by TNBS.

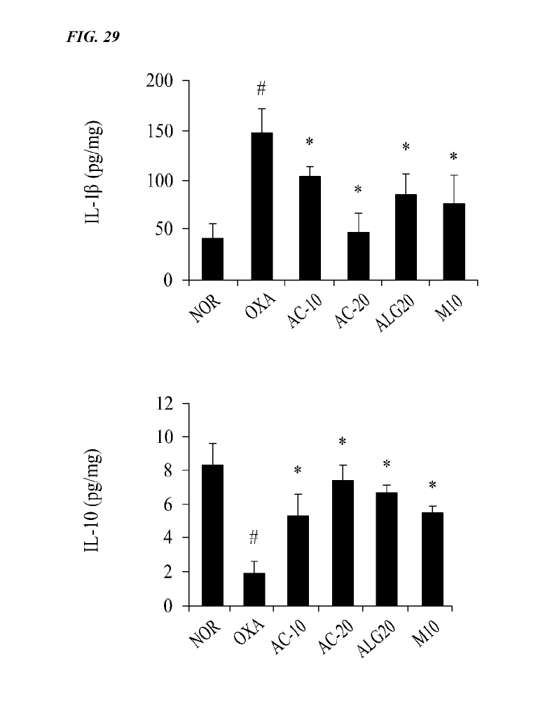

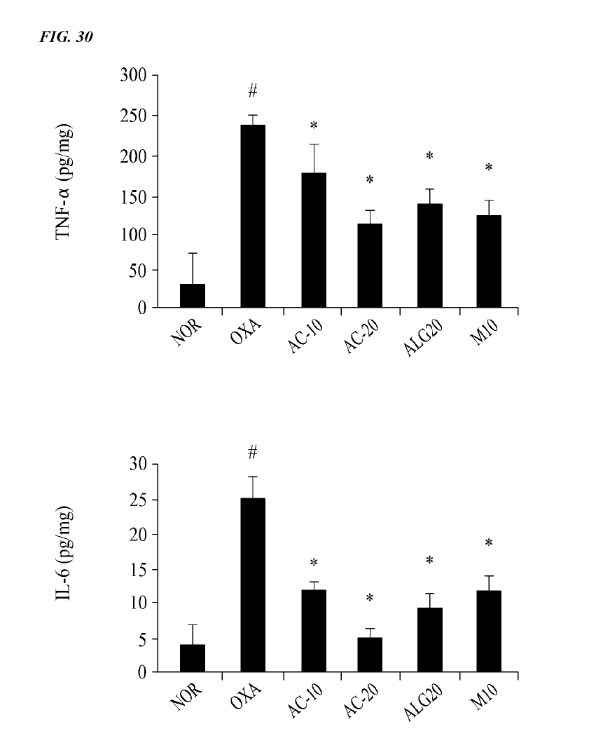

FIG. 29 is the graphs illustrating the expression amounts of IL-1 beta and IL-10 in the colon tissues of the animal model with chronic colitis induced by oxazolone when a combined extract is used as a medicine sample and FIG. 30 is the graphs illustrating the expression amounts of TNF-alpha and IL-6 in the colon tissues of the animal model with chronic colitis induced by oxazolone when a combined extract is used as a medicine sample. In FIGS. 29 and 30, “NOR” represents a normal group, “OXA” represents the experimental group that was orally administered only with normal saline solution after inducing chronic colitis with oxazolone, “AC” represents the combined extract prepared in Preparation Example 33, and “ALG” represents the combined extract prepared in Preparation Example 32. In addition, “AC-10” and “AC-20” respectively represent 10 mg/mouse kg and 20 mg/mouse kg as one time dosage amount of “AC” that is a medicine sample. In addition, in FIGS. 29 and 30, “M” represents mesalazine used as a positive control medicine, and one time dosage amount of mesalazine was 10 mg/mouse kg. As can be seen in FIGS. 29 and 30, the combined extract composed of the fraction of the Anemarrhena asphodeloides Bunge extract and the fraction of the Coptis chinensis extract and the combined extract composed of the fraction of the Anemarrhena asphodeloides Bunge extract, the fraction of the Galla Rhois extract, and the fraction of the Codonopsis lanceolata extract allow the expressions of proinflammatory cytokines to be decreased and the expressions of anti-inflammatory cytokines to be increased in the similar level as mesalazine that was used as a positive control medicine. Especially, the combined extract of the fraction of the Anemarrhena asphodeloides Bunge extract and the fraction of the Coptis chinensis extract exhibits excellent effect as compared with the combined extract composed of the fraction of the Anemarrhena asphodeloides Bunge extract, the fraction of the Galla Rhois extract, and the fraction of the Codonopsis lanceolata extract.

2) Whether or Not Expressions of Inflammation Marker Substances are Suppressed

When a medicine sample was administered into the model animal with chronic colitis induced by oxazolone, the expression amounts of COX-2, iNOS, p65 (NF-Kappa B), p-p65 (phosphor-NF-Kappa B), and β-actin in the colon tissue were measured in the same method as the experiment of model animal with colitis induced by TNBS.

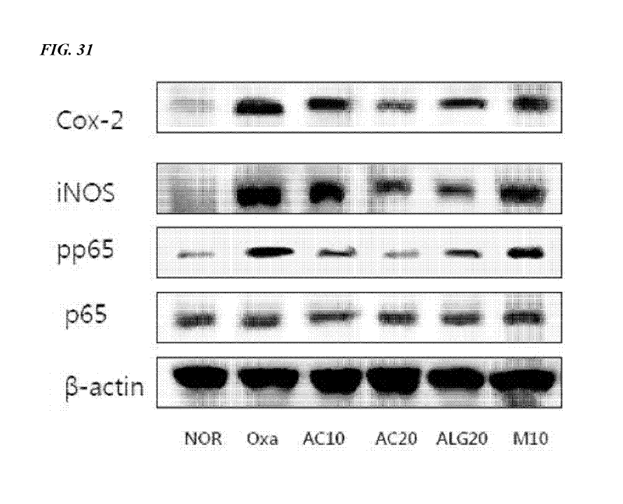

FIG. 31 is the results illustrating the suppressions of the expressions of inflammatory marker substances in the colon tissues of the animal model when a combined extract is administered to the animal model with chronic colitis induced with oxazolone. In FIG. 31, “NOR” represents a normal group, “Oxa” represents the experimental group that was orally administered only with normal saline solution after inducing chronic colitis with oxazolone, “AC” represents the combined extract prepared in Preparation Example 33, and “ALG” represents the combined extract prepared in Preparation Example 32. In addition, “AC10” and “AC20” respectively represent 10 mg/mouse kg and 20 mg/mouse kg as one time dosage amount of “AC” that is a medicine sample. In addition, in FIG. 31, “M” represents mesalazine used as a positive control medicine, and one time dosage amount of mesalazine was 10 mg/mouse kg. As can be seen in FIG. 31, the combined extract composed of the fraction of the Anemarrhena asphodeloides Bunge extract and the fraction of the Coptis chinensis extract and the combined extract composed of the fraction of the Anemarrhena asphodeloides Bunge extract, the fraction of the Galla Rhois extract, and the fraction of the Codonopsis lanceolata extract allow the expressions of COX-2, iNOS, p-p65 (phosphor-NF-Kappa B), and the like to be suppressed in the similar level as mesalazine that was used as a positive control medicine. Especially, the combined extract of the fraction of the Anemarrhena asphodeloides Bunge extract and the fraction of the Coptis chinensis extract exhibits excellent effect as compared with the combined extract composed of the fraction of the Anemarrhena asphodeloides Bunge extract, the fraction of the Galla Rhois extract, and the fraction of the Codonopsis lanceolata extract.

19. Measurement of Effect of Treating Colitis in Test of Model Animal With Chronic Colitis Induced by DSS

(1) Preparation of Experimental Animal

6-week male mice (C57BL/6, 18 to 22 g) were purchased from OrientBio. All the mice were bred under the controlled environmental conditions of 50±10% of humidity and 20 to 22° C. of temperature, and the lighting was repeatedly on for 12 hours and off for 12 hours. A feed for a standard experiment (Samyang, Korea) was used as a feed, and water was freely taken. For all the experiments, one group was 6 mice.

(2) Induction of Chronic Colitis by DSS and Sample Administration

Among the experimental groups, one group was set as a normal group, and for the experimental animals in other groups, chronic colitis was induced with dextran sulfate sodium (Molecular weight:36 to 50 kDaltons). In detail, the animal models with chronic colitis were firstly prepared by taking a 3% (w/v) aqueous dextran sulfate sodium solution for 7 days instead of water as drinking water; again by taking water for 5 days; secondly by taking a 3% (w/v) aqueous dextran sulfate sodium solution for 3 days; and by taking water for 3 days as drinking water. Meanwhile, the normal group was taken with water as drinking water. In addition, from the day that starts the secondly taking with the aqueous dextran sulfate sodium solution, the sample dissolved in normal saline solution was orally administered in the volume that was decided in advance one time a day. At the next day after completing the sample administration, the experimental animals were suffocated to death with carbon dioxide, and the colon from appendix to the region just before the anus was removed from the colon region.

(3) Measurements of Weight Change of Model Animal, Colon Appearance, and Myeloperoxidase (MPO) Activity

1) Analysis of Weight Change Amount

The weight change amounts were analyzed in the same method as the experiment of model animal with colitis induced by TNBS.

2) Appearance Analysis

The appearance of colon was analyzed in the same method as the experiment of model animal with colitis induced by TNBS.

3) Measurement of MPO Activity

The Myeloperoxidase (MPO) activities were measured in the same method as the experiment of model animal with colitis induced by TNBS.

4) Results of Measuring Weight Change Amount, Appearance of Colon, Colon Length, and MPO Activity

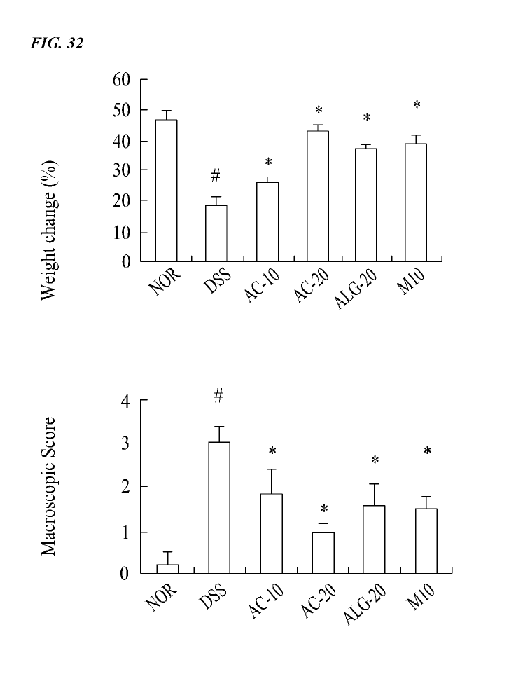

FIG. 32 is the graphs illustrating the weight change and microscopic score of the colon in the animal model with chronic colitis induced by DSS when a combined extract is used as a medicine sample and FIG. 33 is the graphs illustrating the results of the measurements of the colon length and MPO activity in the animal model with chronic colitis induced by DSS when a combined extract is used as a medicine sample. In FIGS. 32 and 33, “NOR” represents a normal group, “AC” represents the combined extract prepared in Preparation Example 33, and “ALG” represents the combined extract prepared in Preparation Example 32. In addition, “AC-10” and “AC-20” respectively represent 10 mg/mouse kg and 20 mg/mouse kg as one time dosage amount of “AC” that is a medicine sample. In addition, in FIGS. 32 and 33, “M” represents mesalazine used as a positive control medicine, and one time dosage amount of mesalazine was 10 mg/mouse kg. As can be seen in FIGS. 32 and 33, the combined extract composed of the fraction of the Anemarrhena asphodeloides Bunge extract and the fraction of the Coptis chinensis extract and the combined extract composed of the fraction of the Anemarrhena asphodeloides Bunge extract, the fraction of the Galla Rhois extract, and the fraction of the Codonopsis lanceolata extract exhibit the effect on suppressing chronic colitis in the similar level as mesalazine that was used as a positive control medicine. Especially, the combined extract of the fraction of the Anemarrhena asphodeloides Bunge extract and the fraction of the Coptis chinensis extract exhibits excellent effect as compared with the combined extract composed of the fraction of the Anemarrhena asphodeloides Bunge extract, the fraction of the Galla Rhois extract, and the fraction of the Codonopsis lanceolata extract.

(4) Analysis of Effect on Expressions of Inflammation Marker Substances

1) Effect on Expressions of Proinflammatory Cytokine and Anti-Inflammatory Cytokine

When a medicine sample was administered into the model animal with chronic colitis induced by DSS, the expression amounts of proinflammatory cytokine and anti-inflammatory cytokine in the colon tissue were measured in the same method as the experiment of model animal with colitis induced by TNBS.

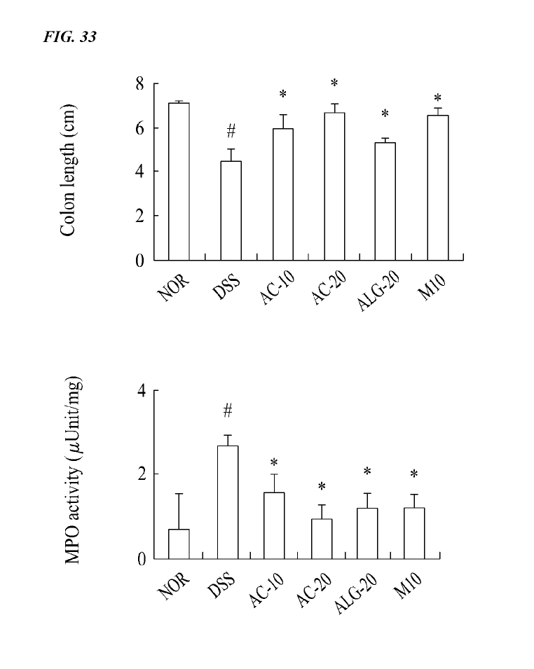

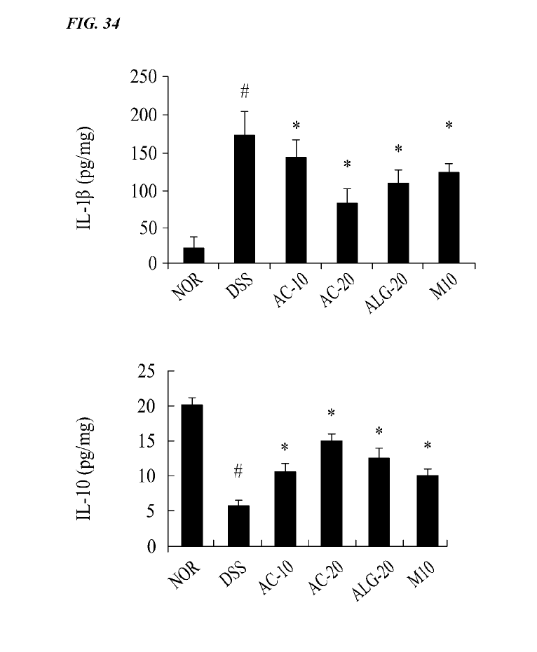

FIG. 34 is the graphs illustrating the expression amounts of IL-1 beta and IL-10 in the colon tissues of the animal model with chronic colitis induced by DSS when a combined extract is used as a medicine sample and FIG. 35 is the graphs illustrating the expression amounts of TNF-alpha and IL-6 in the colon tissues of the animal model with chronic colitis induced by DSS when a combined extract is used as a medicine sample. In FIGS. 34 and 35, “NOR” represents a normal group, “AC” represents the combined extract prepared in Preparation Example 33, and “ALG” represents the combined extract prepared in Preparation Example 32. In addition, “AC-10” and “AC-20” respectively represent 10 mg/mouse kg and 20 mg/mouse kg as one time dosage amount of “AC” that is a medicine sample. In addition, in FIGS. 34 and 35, “M” represents mesalazine used as a positive control medicine, and one time dosage amount of mesalazine was 10 mg/mouse kg. As can be seen in FIGS. 34 and 35, the combined extract composed of the fraction of the Anemarrhena asphodeloides Bunge extract and the fraction of the Coptis chinensis extract and the combined extract composed of the fraction of the Anemarrhena asphodeloides Bunge extract, the fraction of the Galla Rhois extract, and the fraction of the Codonopsis lanceolata extract allow the expressions of proinflammatory cytokines to be decreased and the expressions of anti-inflammatory cytokines to be increased in the similar level as mesalazine that was used as a positive control medicine. Especially, the combined extract of the fraction of the Anemarrhena asphodeloides Bunge extract and the fraction of the Coptis chinensis extract exhibits excellent effect as compared with the combined extract composed of the fraction of the Anemarrhena asphodeloides Bunge extract, the fraction of the Galla Rhois extract, and the fraction of the Codonopsis lanceolata extract.

2) Whether or Not Expressions of Inflammation Marker Substances are Suppressed

When a medicine sample was administered into the model animal with chronic colitis induced by DSS, the expression amounts of COX-2, iNOS, p65 (NF-Kappa B), p-p65 (phosphor-NF-Kappa B), and β-actin in the colon tissue were measured in the same method as the experiment of model animal with colitis induced by TNBS.

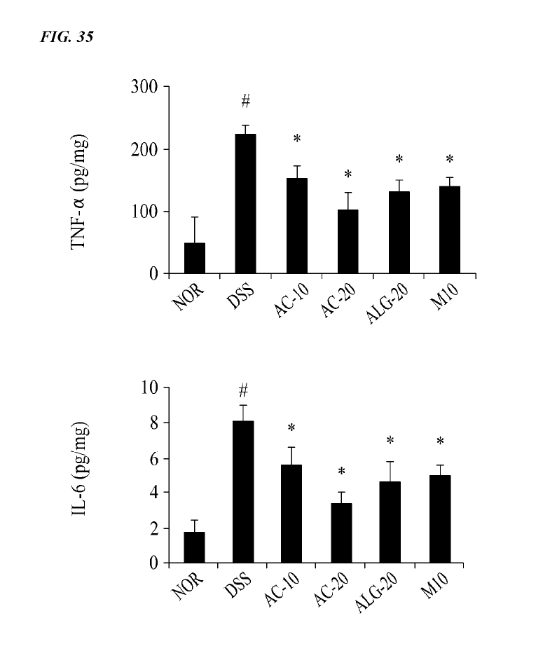

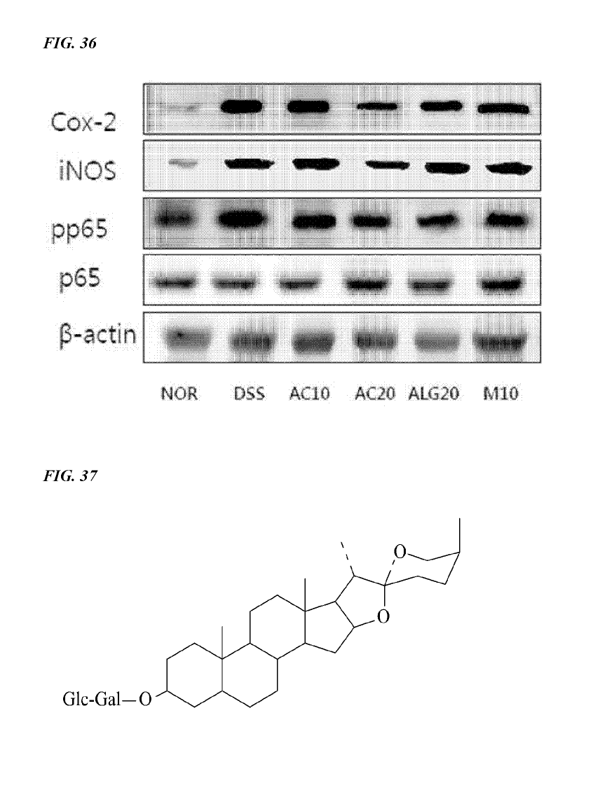

FIG. 36 is the results illustrating the suppressions of the expressions of inflammatory marker substances in the colon tissues of the animal model when a combined extract is administered to the animal model with chronic colitis induced with DSS. In FIG. 36, “NOR” represents a normal group, “AC” represents the combined extract prepared in Preparation Example 33, and “ALG” represents the combined extract prepared in Preparation Example 32. In addition, “AC10” and “AC20” respectively represent 10 mg/mouse kg and 20 mg/mouse kg as one time dosage amount of “AC” that is a medicine sample. In addition, in FIG. 36, “M” represents mesalazine used as a positive control medicine, and one time dosage amount of mesalazine was 10 mg/mouse kg. As can be seen in FIG. 36, the combined extract composed of the fraction of the Anemarrhena asphodeloides Bunge extract and the fraction of the Coptis chinensis extract and the combined extract composed of the fraction of the Anemarrhena asphodeloides Bunge extract, the fraction of the Galla Rhois extract, and the fraction of the Codonopsis lanceolata extract allow the expressions of COX-2, iNOS, p-p65 (phosphor-NF-Kappa B), and the like to be suppressed in the similar level as mesalazine that was used as a positive control medicine. Especially, the combined extract of the fraction of the Anemarrhena asphodeloides Bunge extract and the fraction of the Coptis chinensis extract exhibits excellent effect as compared with the combined extract composed of the fraction of the Anemarrhena asphodeloides Bunge extract, the fraction of the Galla Rhois extract, and the fraction of the Codonopsis lanceolata extract.

IV. 4th Experiment:Confirmation of Effect of Timosaponin A-III on Preventing or Treating Colitis

20. Preparation of Timosaponin A-III Isolated from Anemarrhena asphodeloides Bunge

### PREPARATION EXAMPLE 34

### Preparation of Timosaponin A-III Isolated From Anemarrhena asphodeloides Bunge

After an Anemarrhena asphodeloides Bunge was well dried as it is, or was skinned and then well dried, the dried Anemarrhena asphodeloides Bunge was extracted in a water bath using 70% ethanol, and then was completely dried to prepare a 70% ethanol extract. After re-suspending the obtained 70% ethanol extract of the Anemarrhena asphodeloides Bunge in water, the re-suspended Anemarrhena asphodeloides Bunge was extracted with CH₂Cl₂; and a water layer was extracted with BuOH, and then concentrated under the reduced pressure. The decompressed concentrated BuOH extract was re-crystallized with MeOH, and the crystallized precipitate was washed with MeOH to obtain a methanol-insoluble fraction and a methanol-soluble fraction. Since then, the methanol-soluble fraction was subjected to a silica gel chromatography (CH₂Cl₂:MeOH:H₂O=7:1:0.5→7:2:0.5→7:3:1) using a mixed solvent of methylenechloride, methanol, and water to isolate Timosaponin A-III, Timosaponin B-II, Timosaponin B-III, and Timosaponin D. Whether or not Timosaponin A-III was isolated was confirmed with a mass analysis and ¹³C-NMR (Bruker, AVANCE digital 400). FIG. 37 illustrates Chemical Structural Formula of Timosaponin A-III.

<Timosaponin A-III:White Amorphous Powder>

FAB-MS m/z:763.5 [M+Na]⁺

¹³C NMR (125 MHz) 30.971 (C-1). 27.031 (C-2), 75.522 (C-3), 30.971 (C-4), 36.963 (C-5), 26.833 (C-6), 26.833 (C-7), 35.557 (C-8), 40.260 (C-9), 35.278 (C-10), 21.174 (C-11), 40.341 (C-12), 42.503 (C-13), 56.483 (C-14), 32.183 (C-15), 81.376 (C-16), 62.216 (C-17), 16.622 (C-18), 24.040 (C-19), 40.907 (C-20), 14.941 (C-21), 109.7 (C-22), 26.434 (C-23), 26.229 (C-24), 27.573 (C-25), 69.882 (C-26), 16.308 (C-27), 102.588 (3-Gal C-1′), 81.879 (C-2′), 75.263 (C-3′), 71.753 (C-4′), 76.650 (C-5′), 62.824 (C-6′), 106.167 (0-3 Glu C-1″), 76.987 (C-2″), 78.459 (C-3″), 72.987 (C-4″), 78.064 (C-5″), 65.129 (C-6″)

21. Measurement of Effect of Treating Colitis Through Test of Animal Model With Induced Colitis

(1) Preparation of Experimental Animal

4-week ICR male mice (24 to 27 g) were purchased from OrientBio. All the mice were bred under the controlled environmental conditions of 50±10% of humidity and 25±2° C. of temperature, and the lighting was repeatedly on for 12 hours and off for 12 hours. A feed for a standard experiment (Samyang, Korea) was used as a feed, and water was freely taken. For all the experiments, one group was 6 mice.

(2) Induction of acute colitis by TNBS and sample administration Among the experimental groups, one group was set as a normal group, and for the experimental animals in other groups, colitis was induced with 2,4,6-trinitrobenzenesulfonic acid (TNBS). In detail, after lightly anesthetizing the experimental animals with ether, 0.1 ml of the solution prepared by mixing 2.5 g of 2,4,6-Trinitrobenzene sulfonic acid (NBS) solution with 50% ethanol was injected into the colon through the anus using a syringe having 1 ml volume and oval point, respectively; and then, the syringe was vertically picked up, and then, maintained for 30 seconds to induce the inflammation. Meanwhile, 0.1 ml of normal saline solution was orally administered to the normal group. Since then, from the next day, the sample dissolved in normal saline solution was orally administered in the volume that was decided in advance one time a day for 3 days. At the next day after completing the sample administration, the experimental animals were suffocated to death with carbon dioxide, the colon from appendix to the region just before the anus was removed from the colon region.

(3) Measurements of Weight Change, Appearance of Colon and Myeloperoxidase (MPO) Activity

1) Analysis of Weight Change Amount

The sample administration to the model animals with acute colitis induced by TNBS was stopped, and at the next day, the weights of the experimental animals were measured, and then, the measured weights were compared with the initial weights to calculate the change amounts of weights

2) Appearance Analysis

The scores about the removed colons were estimated by observing the appearances and the lengths of the colons according to the following criteria (Hollenbach, and the like, the criteria about the level of colon in 2005) as listed in Table 13. At this time, as a positive control group, the group administered with Mesalazine (Sigma) was used. In addition, some of the content in the colon was collected in order to analyze the rumen microorganisms, and then, was stored in a freezer of -80° C. From the tissue of colon, the content in the colon was completely removed and then the tissue of colon was washed with normal saline solution. Since then, some of the tissue was fixed with 4% formaldehyde fixing solution in order to be used as a sample for a histopathological examination, and the rest was stored in a freezer of −80° C. for a molecular biological analysis.

TABLE 13

| Macroscopic | |
|---|---|
| score | Criteria |
| 0 | There are no any kinds of ulcers and inflammations. |
| 1 | There are hyperemias without blood. |
| 2 | There are ulcers with hyperemias |
| 3 | The ulcer and inflammation were found only on one region. |
| 4 | The ulcers and inflammations were found on two or more |
| | regions. |
| 5 | The ulcer was enlarged in the size of 2 cm or more. |

3) Myeloperoxidase (MPO) Activity Measurement

200 μl of lysis buffer was added to 100 mg of colon tissue, and then, the colon tissue was homogenized. Since then, the homogenized colon tissue was centrifuged under the conditions of 4° C. and 13000 rpm for 15 minutes, and then, the supernatant thereof was obtained. In the supernatant, the MPO activity was measured using a mouse MPO assay ELISA kit (Hbt HK210, USA). 100 μl of the supernatant was added in a 96 well plate, and then, reacted at room temperature for 1 hour. After completing the reaction, the plate was overturned and emptied; was repeatedly washed three times with 200 μl of washing buffer solution; 100 μl of diluted tracer was added thereto; and then the reaction was performed at room temperature for 1 hour. After completing the reaction, the plate was overturned and emptied; and then each well in the plate was washed using 200 μl of washing buffer solution. After repeatedly performing the washing processes three times using 200 μl of washing buffer solution, 100 μl of diluted streptavidin-peroxidase conjugate was added thereto, and then, the reaction was performed at room temperature for 1 hour. After completing the reaction, the plate was overturned and emptied, and then, each well in the plate was washed with 200 μl of washing buffer solution. After the washing processes were repeatedly performed three times using 200 μl of washing buffer solution, 100 μl of TMB substrate solution was added thereto; the plate was wrapped in an aluminum foil to block out the light; and then, the reaction was performed at room temperature for 30 minutes. Since then, 100 μl of a stop solution was added to stop the reaction, and then, the absorbance was measured at 450 nm using an ELISA reader.

4) Results of Measuring Weight Change Amount, Appearance of Colon, Length of Colon, and MPO Activity

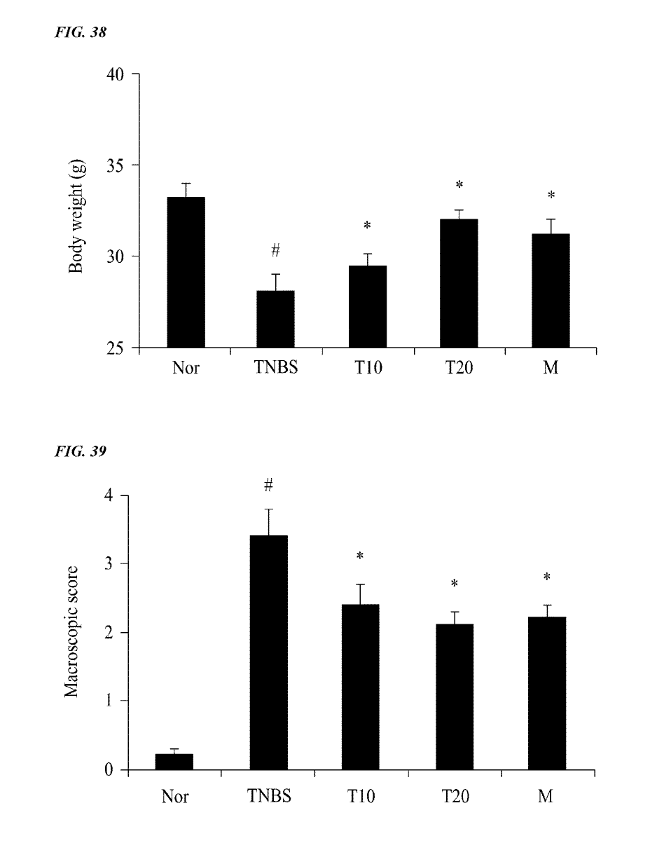

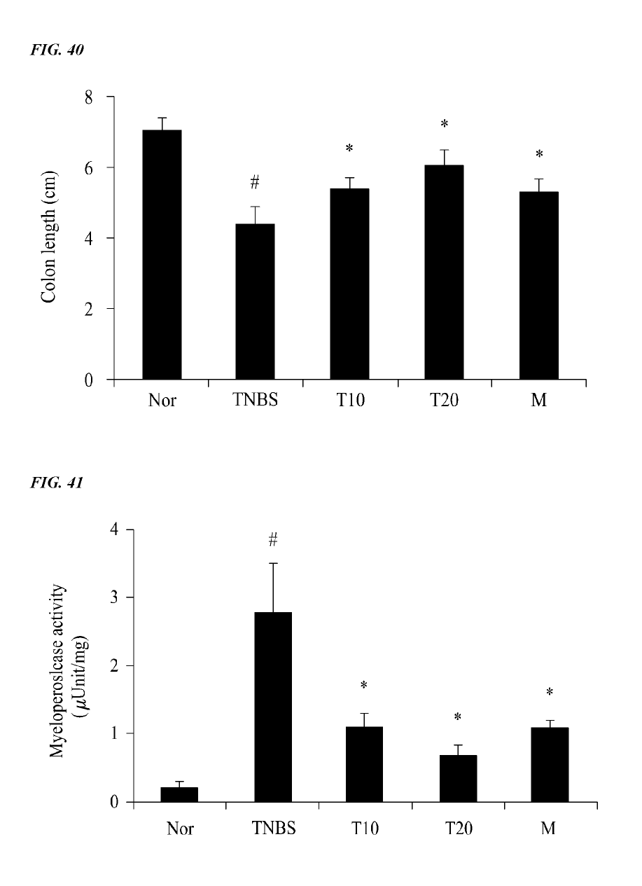

FIG. 38 is the graph illustrating the weight change of the animal model with acute colitis induced by TNBS when Timosaponin A-III is used in Preparation Example 34 as a medicine sample and FIG. 39 is the graph illustrating the macroscopic score of the colon isolated from the animal model with acute colitis induced by TNBS when Timosaponin A-III is used in Preparation Example 34 as a medicine sample. FIG. 40 is the results illustrating the colon length of the animal model with acute colitis induced with TNBS when Timosaponin A-III is used in Preparation Example 34 as a medicine sample and FIG. 41 is the results illustrating the measurement of MPO activity of the colon isolated from the animal model with acute colitis induced with TNBS when Timosaponin A-III is used in Preparation Example 34 as a medicine sample. In FIGS. 38 to 41, “Nor” was the normal group, “TNBS” was the group, in which normal saline solution instead of a specific medicine sample was supplied to the model animal with acute colitis induced by TNBS, and “T” was Timosaponin A-III. In addition, “T10” and “T20” were 10 mg/mouse kg and 20 mg/mouse kg as one time dosage amount of “T”, that is, a medicine sample, respectively. In addition, in FIGS. 38 to 41, “M” means mesalazine used as a positive control medicine, and one time dosage amount of mesalazine was 10 mg/mouse kg. As can be seen in FIGS. 38 to 41, Timosaponin A-III exhibits the effect on alleviating or improving acute colitis induced by TNBS according to the concentration dependent. In addition, the effect of Timosaponin A-III on preventing or treating colitis was the similar level as mesalazine that was used as a positive control medicine.

V. Preparations of Pharmaceutical Composition and Food Composition

22. Preparation of Pharmaceutical Composition Including Anemarrhena asphodeloides Bunge Extract and the Like

In the following preparations of pharmaceutical compositions, the Anemarrhena asphodeloides Bunge extract may be substituted with a butanol-soluble fraction of the Anemarrhena asphodeloides Bunge extract, Mangiferin, Neomangiferin, the combined extract of Preparation Example 14, the combined extract of Preparation Example 20, the combined extract of Preparation Example 33, and Timosaponin A-III of Preparation Example 34.

<22-1> Preparation of Powders

2 g of the Anemarrhena asphodeloides Bunge extract of Preparation Example 1 1 g of lactose

The above ingredients were mixed, and then the mixed ingredients were filled in airtight container to prepare powders.

<22-2> Preparation of Tablet

100 mg of the Anemarrhena asphodeloides Bunge extract of Preparation Example 1

100 mg of cornstarch

100 mg of lactose

2 mg of magnesium stearate

The above ingredients were mixed, and then the mixed ingredients were subjected to a tableting according to a general method for preparing a tablet to prepare a tablet.

<22-3> Preparation of Capsules

100 mg of the Anemarrhena asphodeloides Bunge extract of Preparation Example 1

100 mg of cornstarch

100 mg of lactose

2 mg of magnesium stearate

The above ingredients were mixed, and then the mixed ingredients were filled in a gelatin capsule according to a general method for preparing a capsule to prepare a capsule.

<22-4> Preparation of Pill

1 g of the Anemarrhena asphodeloides Bunge extract of Preparation Example 1 1.5 g of lactose

1 g of glycerin

0.5 g of xylitol

The above ingredients were mixed, and then the mixed ingredients were prepared to be 4 g per a pill according to a general method.

<22-5> Preparation of Granule

150 mg of the Anemarrhena asphodeloides Bunge extract of Preparation Example 1

50 mg of soybean extract

200 mg of glucose

600 mg of starch

The above ingredients were mixed; the mixed ingredients were added with 100 mg of 30% ethanol; the solution thus obtained was dried at 60° C. to prepare a granule; and the granule was filled in a bag.

<22-6> Preparation of Injections

100 mg of the Anemarrhena asphodeloides Bunge extract of Preparation Example 1

3.0 mg of sodium meta bisulfite

0.8 mg of methylparaben

0.1 mg of propylparaben

Suitable amount of sterile distilled water for injection

The above ingredients were mixed; the mixed ingredients were filled in a 2 ml ample; and then, the ample thus obtained was sterilized to prepare injections.

23. Preparation of Food Composition Including Anemarrhena asphodeloides Bunge Extract and the Like

In the following preparations of food compositions, the Anemarrhena asphodeloides Bunge extract may be substituted with a butanol-soluble fraction of the Anemarrhena asphodeloides Bunge extract, Mangiferin, Neomangiferin, the combined extract of Preparation Example 14, the combined extract of Preparation Example 20, the combined extract of Preparation Example 33, and Timosaponin A-III of Preparation Example 34.

<23-1> Preparation of Wheat Flour Food

0.5 parts by weight to 5.0 parts by weight of the Anemarrhena asphodeloides Bunge extract of Preparation Example 1 according to the present invention was added to wheat flour, and then, the mixture thus obtained was used to prepare bread, cake, cookies, cracker, and noodles.

<23-2> Preparation of Soup and Gravies

0.1 parts by weight to 5.0 parts by weight of the Anemarrhena asphodeloides Bunge extract of Preparation Example 1 according to the present invention was added to soup and gravies to prepare a livestock processed product, the soup for noodles, and gravies for the promotion of health.

<23-3> Preparation of Ground Beef

10 parts by weight of the Anemarrhena asphodeloides Bunge extract of Preparation Example 1 according to the present invention was added to a ground beef to prepare a ground beef for the promotion of health.

<23-4> Preparation of Dairy Products

5 parts by weight to 10 parts by weight of the Anemarrhena asphodeloides Bunge extract of Preparation Example 1 according to the present invention was added to milk, and then the milk thus obtained was used to prepare various dairy products, such as, butter and ice cream.

<23-5> Preparation of Sunsik Product

Brown rice, barley, glutinous rice, and adlay were dried by pregelatinizing them according to the known methods, and then, roasted. Since then, the mixture was prepared to have a particle size of 60 meshes using a grinder.

Black beans, black sesame, and perilla were steamed and dried according to the known method, and were roasted. Since then, the mixture thus obtained was prepared to have a particle size of 60 meshes using a grinder.

The above-prepared gains, seeds nuts, and the Anemarrhena asphodeloides Bunge extract of Preparation Example 1 were mixed in the following ratio.

Grains (30 parts by weight of brown rice, 15 parts by weight of adlay, and 20 parts by weight of barley)

Seeds nuts (7 parts by weight of perilla, 8 parts by weight of black beans, and 7 parts by weight of black sesame)

Anemarrhena asphodeloides Bunge extract of Preparation Example 1 (3 parts by weight)

Lingzhi mushroom (0.5 parts by weight)

The foxglove (0.5 parts by weight)

<23-6> Preparation of Health Drink

The minor ingredients, such as, fructose (0.5 g), oligosaccharide (2 g), sugar (2 g), table salt (0.5 g), and water (75 g) were homogeneously combined with 5 g of the Anemarrhena asphodeloides Bunge extract of Preparation Example 1 according to the present invention, and then, the mixture thus obtained was instantly sterilized. Since then, the sterilized mixture was packaged in a small container, such as, a glass bottle and a plastic bottle.

<23-7> Preparation of Vegetable Juice

5 g of the Anemarrhena asphodeloides Bunge extract of Preparation Example 1 according to the present invention was added to 1,000 ml of tomato or carrot juice to prepare vegetable juice.

<23-8> Preparation of Fruit Juice

1 g of the Anemarrhena asphodeloides Bunge extract of Preparation Example 1 according to the present invention was added to 1,000 ml of apple or grape juice to prepare fruit juice.

24. Preparation of Feed Additives Including Anemarrhena asphodeloides Bunge Extract

In the following preparations of feed additives, the Anemarrhena asphodeloides Bunge extract may be substituted with a butanol-soluble fraction of the Anemarrhena asphodeloides Bunge extract, Mangiferin, Neomangiferin, the combined extract of Preparation Example 14, the combined extract of Preparation Example 20, the combined extract of Preparation Example 33, and Timosaponin A-III of Preparation Example 34.

<24-1>Feed Additive 1

0.1 to 20 parts by weight of the Anemarrhena asphodeloides Bunge extract of Preparation Example 1, 0.001 to 0.01 parts by weight of lipase, 1 to 20 parts by weight of calcium phosphate, 0.01 to 0.1 parts by weight of vitamin E, 1 to 10 parts by weight of enzyme powder, 0.1 to 10 parts by weight of lactobacillus, 0.01 to 10 parts by weight of bacillus culture solution, and 20 to 90 parts by weight of glucose were mixed to prepare a feed additive.

<24-2>Feed Additive 2

24 parts by weight of the Anemarrhena asphodeloides Bunge extract of Preparation Example 1, 24 parts by weight of Houttuynia cordata extract powder, 1 part by weight of lactobacillus, 10 parts by weight of yeast, 20 parts by weight of glucose, and 20 parts by weight of alfalfa powder were mixed to prepare a feed additive.

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

1. A method for preventing or treating colitis, comprising administering to a subject in need thereof an effective amount of a C3 to C8-alcohol-soluble fraction of an Anemarrhena asphodeloides Bunge extract or a composition comprising a C3 to C8-alcohol-soluble fraction of an Anemarrhena asphodeloides Bunge extract as an active ingredient,
wherein the extraction solvent for the Anemarrhena asphodeloides Bunge extract is selected from water, a C1 to C2 alcohol, or a mixed solvent thereof.

2. The method of claim 1, the composition further comprising any one selected from a Galla Rhois extract, a C3 to C8-alcohol-soluble fraction of the Galla Rhois extract, or 1,2,3,4,6-penta-O-galloyl-β-D-glucose, as an active ingredient,
wherein the extraction solvent for the Galla Rhois extract is selected from water, a C1 to C2 alcohol, or a mixed solvent thereof.

3. The method of claim 1, the composition further comprising any one selected from a Codonopsis lanceolata extract, a C3 to C8-alcohol-soluble fraction of the Codonopsis lanceolata extract, or Lancemaside A, as an active ingredient,
wherein the extraction solvent for the Codonopsis lanceolata extract is selected from water, a C1 to C2 alcohol, or a mixed solvent thereof.

4. The method of claim 1, the composition further comprising any one selected from a Galla Rhois extract, a C3 to C8-alcohol-soluble fraction of the Galla Rhois extract, or 1,2,3,4,6-penta-O-galloyl-β-D-glucose; and any one selected from a Codonopsis lanceolata extract, a C3 to C8-alcohol-soluble fraction of the Codonopsis lanceolata extract, or Lancemaside A, as an active ingredient,
wherein the extraction solvent for the Galla Rhois extract is selected from water, a C1 to C2 alcohol, or a mixed solvent thereof and
the extraction solvent for the Codonopsis lanceolata extract is selected from water, a C1 to C2 alcohol, or a mixed solvent thereof.

5. The method of claim 1, the composition further comprising any one selected from a Coptis extract or a C3 to C8-alcohol-soluble fraction of the Coptis extract, as an active ingredient,
wherein the extraction solvent for the Coptis extract is selected from water, a C1 to C2 alcohol, or a mixed solvent thereof.

6. The method of claim 1, wherein the alcohol-soluble fraction of the Anemarrhena asphodeloides Bunge extract is obtained by:
adding water to the Anemarrhena asphodeloides Bunge extract;
suspending the Anemarrhena asphodeloides Bunge extract in water;
adding a C3 to C8 alcohol; and
then, being subjected to a fractionation thereof.

7. The method of claim 1, wherein the alcohol-soluble fraction of the Anemarrhena asphodeloides Bunge extract is a butanol-soluble fraction of the Anemarrhena asphodeloides Bunge extract.

8. The method of claim 1, wherein the Anemarrhena asphodeloides Bunge extract or the alcohol-soluble fraction of the Anemarrhena asphodeloides Bunge extract includes Mangiferin or Neomangiferin.

9. The method of claim 2, wherein the alcohol-soluble fraction of the Galla Rhois extract is obtained by:
adding water to the Galla Rhois extract;
suspending the Galla Rhois extract in water;
adding a C3 to C8 alcohol; and
then, being subjected to a fractionation thereof.

10. The method of claim 2, wherein the alcohol-soluble fraction of the Galla Rhois extract is a butanol-soluble fraction of the Galla Rhois extract.

11. The method of claim 2, wherein the Galla Rhois extract or the alcohol-soluble fraction of the Galla Rhois extract includes 1,2,3,4,6-penta-O-galloyl-β-D-glucose.

12. The method of claim 3, wherein the alcohol-soluble fraction of the Codonopsis lanceolata extract is obtained by:
adding water to the Codonopsis lanceolata extract;
suspending the Codonopsis lanceolata extract in water;
adding a C3 to C8 alcohol; and
then, being subjected to a fractionation thereof.

13. The method of claim 3, wherein the alcohol-soluble fraction of the Codonopsis lanceolata extract is a butanol-soluble fraction of the Codonopsis lanceolata extract.

14. The method of claim 3, wherein the Codonopsis lanceolata extract or the alcohol-soluble fraction of the Codonopsis lanceolata extract includes Lancemaside A.

15. The method of claim 4, wherein the alcohol-soluble fraction of the Galla Rhois extract is obtained by:
adding water to the Galla Rhois extract;
suspending the Galla Rhois extract in water;
adding a C3 to C8 alcohol; and
then, being subjected to a fractionation thereof; and
the alcohol-soluble fraction of the Codonopsis lanceolata extract is obtained by:
adding water to the Codonopsis lanceolata extract;
suspending the Codonopsis lanceolata extract in water;
adding a C3 to C8 alcohol; and
then, being subjected to a fractionation thereof.

16. The method of claim 4, wherein the alcohol-soluble fraction of the Galla Rhois extract is a butanol-soluble fraction of the Galla Rhois extract; and
the alcohol-soluble fraction of the Codonopsis lanceolata extract is a butanol-soluble fraction of the Codonopsis lanceolata extract.

17. The method of claim 4, wherein the Galla Rhois extract or the alcohol-soluble fraction of the Galla Rhois extract includes glucose1,2,3,4,6-penta-O-galloyl-β-D-glucose; and the Codonopsis lanceolata extract or the alcohol-soluble fraction of the Codonopsis lanceolata extract includes Lancemaside A.

18. The method of claim 5, wherein the alcohol-soluble fraction of the Coptis extract is obtained by:
adding water to the Coptis extract;
suspending the Coptis extract in water; adding a C3 to C8 alcohol; and
then, being subjected to a fractionation thereof.

19. The method of claim 5, wherein the alcohol-soluble fraction of the Coptis extract is a butanol-soluble fraction of the Coptis extract.

20. The method of claim 5, wherein the Coptis extract or the alcohol-soluble fraction of the Coptis extract includes Berberine.
